(19) United States
(12) Patent Application Publication
(10) Pub. No.: US 2024/0239804 A1
(43) Pub. Date: Jul. 18, 2024

(54) FUROINDAZOLE DERIVATIVES AS ANTAGONISTS OR INHIBITORS OF GPR84

(71) Applicant: Bayer Aktiengesellschaft, Leverkusen (DE)

(72) Inventors: Olaf PANKNIN, Hindås (SE); Frank SACHER, Berlin (DE); Gernot LANGER, Falkensee (DE); Katrin NOWAK-REPPEL, Berlin (DE); Reinhard NUBBEMEYER, Berlin (DE); Sabine PILARI, Berlin (DE); Antje ROTTMANN, Berlin (DE); Holger SIEBENEICHER, Berlin (DE)

(21) Appl. No.: 18/557,139

(22) PCT Filed: Apr. 25, 2022
(86) PCT No.: PCT/EP2022/060833

§ 371 Date: Oct. 25, 2023

(30) Foreign Application Priority Data
Apr. 29, 2021 (EP) 21171084.3

(51) Int. Cl. C07D 491/048; C07D 519/00

(52) U.S. Cl. CPC C07D 491/048; C07D 519/00

(57) ABSTRACT

The present invention covers furoindazole compounds of general formula (I):

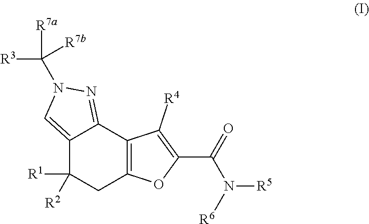

in which R¹, R², R³, R⁴, R⁵, R⁶, R^{7a} and R^{7b} are as defined herein, methods of preparing said compounds, intermediate compounds useful for preparing said compounds, pharmaceutical compositions comprising said compounds and the use of said compounds for manufacturing pharmaceutical compositions for the treatment or prophylaxis of diseases, in particular of autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

The present invention covers furoindazole compounds of general formula (I) as described and defined herein, methods of preparing said compounds, intermediate compounds useful for preparing said compounds, pharmaceutical compositions comprising said compounds, and the use of said compounds for manufacturing pharmaceutical compositions for the treatment or prophylaxis of diseases, in particular of autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders.

### BACKGROUND

The present invention covers furoindazole compounds of general formula (I) which are antagonists of the G-protein coupled receptor 84 (also known as GPR84). The relevance of GPR84 for human disease has been described and studied in several publications.

Medium-chain free fatty acids (MCFFAs) are fatty acids with tails of 6 to 12 carbons and can activate GPR84 (Wang J et al., J. Biol. Chem. 2006 Nov. 10, 281(45): 34457-64). There are two sources of FAs for animal metabolism, exogenously-derived (dietary) FAs and endogenously-synthesized FAs. The biosynthesis of the latter is catalysed by FASN. MCFFAs stimulate release of IL6 from fibroblasts (Smith and Tasi, Nat. Prod. Rep. 2007 October, 24(5): 1041-72) and myristic acid increases IL6 and IL8 levels in human coronary arterial smooth muscle (HCASM) and endothelial (HCEC) cells (Soto-Vaca A. et al., J. Agric. Food Chem. 2013 Oct. 23, 61(42): 10074-9).

GPR84 belongs to the group of Free Fatty Acid (FFA) receptors (Wang J. et al., J. Biol. Chem. 2006 Nov. 10, 281(45): 34457-64). The group of FFA receptors consists of 4 GPCRs (FFA1-FFA2) and the new members GPR42 and GPR84. FFA receptors are involved in biological processes such as metabolic and immune function receptors (Wang J. et al., J. Biol. Chem. 2006 Nov. 10, 281(45): 34457-64).

In contrast to all other FFA receptors which have a broader expression pattern, GPR84 has been described to be expressed primarily in various leukocyte populations and adipocytes (Wang J. et al., J. Biol. Chem. 2006 Nov. 10, 281(45): 34457-64; Lattin J. E. et al., Immunome Res. 2008 Apr. 29, 4: 5; Nagasaki H. et al., FEBS Lett. 2012 Feb. 17, 586(4): 368-72).

Activation of GPR84 promotes a comprehensive fibrotic and inflammatory cellular response, exerted by enhanced migration of macrophages and neutrophils, promoted pro-inflammatory M1 macrophage polarization and response and secretion of key inflammatory cytokines such as IL1beta and TNFalpha (Gagnon L. et al., Am. J. Pathol. 2018 May, 188(5): 1132-1148; Muredda L. et al., Arch. Physiol. Biochem. 2018 May, 124(2): 97-108; Huang Q. et al., Dev. Comp. Immunol. 2014, 45(2): 252-258). Based on the involvement of GPR84 in fibrotic and inflammatory cellular response several diseases have been suggested to be GPR84 dependent.

GPR84 as microglia-associated protein is expressed in neuroinflammatory conditions and is described as a potential target for the treatment of multiple sclerosis (Bouchard C. et al., Glia 2007 June, 55(8): 790-800) and for endometriosis associated and inflammatory pain (Sacher F. et al. 2018, Conference Abstract SRI 2018). Furthermore, inhibition of activity and/or the knockout of GPR84 are also effective in the treatment of neuropathic pain in several preclinical models (Roman et al. 2010, 7th Forum of European Neuroscience (FENS)).

The relevance of GPR84 for inflammatory kidney diseases has been shown in experiments using Gpr84-knockout mice or GPR84 antagonist in models of kidney fibrosis and models for inflammatory liver diseases like non-alcoholic, alcoholic- and toxic fatty liver diseases (Puengel et al. 2018, 2018 International Liver Congress (ILC) of the European Association for the Study of the Liver (EASL); Thibodeau J. F. et al. 2018, 51st Annual Meeting and Exposition of the American Society of Nephrology (ASN): Kidney Week 2018).

As described previously for macrophages and monocytes, inflammatory changes in adipose tissue enhance expression of GPR84 in adipocytes and modulation of GPR84 regulates adipocyte immune response capabilities (Muredda et al., Archives of Physiology and Biochemistry 2017 August, 124(2): 1-12) indicating the relevance of GPR84 in metabolic and metabolic-endocrine disorders like metabolic syndrome, insulin resistance, diabetes mellitus type I and type II, and polycystic ovary syndrome (PCOS) through normalization of adipose tissue inflammation.

Regulation of neutrophil activity and general inflammation by GPR84 was also described to be relevant for lung diseases like asthma, idiopathic pulmonary fibrosis and chronic obstructive pulmonary disease (Nguyen et al. 2018; Annual Congress Scientific Sessions of the American Heart Association (AHA 2018); Saniere L. et al. 2019; 2019 International Conference of the American Thoracic Society (ATS)).

Few compounds are known as GPR84 antagonists, for example the patent applications WO2013092791 and WO2014095798 disclose dihydropyrimidinoisoquinolinones having activity as GPR84 antagonists. Such compounds find utility in several therapeutic applications including inflammatory conditions.

The patent applications WO2015197550 and WO2016169911 disclose related dihydropyridoisoquinolinones as GPR84 antagonists.

The patent application WO2018161831 discloses dibenzoannulen hydrogen phosphates as GPR84 antagonists.

The patent application WO2009023773 discloses galactokinase inhibitors that were identified by a high throughput screening approach. Among the identified hits were two furoindazole compounds.

The patent application US20090163545 discloses compounds for altering the lifespan of eukaryotic organisms that were identified by a cell-based phenotypic high throughput screening approach. Among the identified hits were two furoindazole compounds.

The patent applications U.S. Pat. No. 6,245,796B1, WO2001083487 and WO2011071136 disclose aromatic tricyclic pyrrole or pyrazole derivatives as 5-HT2c ligands.

The patent application WO2016085990 discloses compounds inhibiting serine hydroxy-methyltransferase 2 activity that were identified by a high throughput screening approach. Among the identified hits were nine furoindazole compounds.

The patent application WO2019084271 discloses compounds inhibiting the non-canonical poly(A) RNA polymerase associated domain containing protein 5 (PAPD5) originating from diverse compound classes that were identified by a high throughput screening approach. Among the identified hits were eight furoindazole compounds.

However, the state of the art does not describe the furoindazole compounds of general formula (I) of the present invention as described and defined herein.

It has now been found, and this constitutes the basis of the present invention, that the compounds of the present invention have surprising and advantageous properties.

In particular, the compounds of the present invention have surprisingly been found to be effective antagonists of human GPR84 and may be used for the treatment or prophylaxis of diseases, in particular of autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders.

### DESCRIPTION

In accordance with a first aspect, the present invention covers compounds of general formula (I):

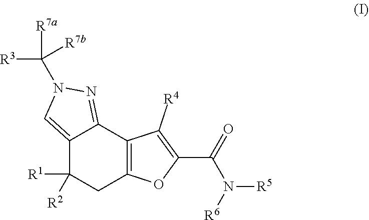

in which:
R¹ represents hydrogen, C₁-C₄-alkyl or C₁-C₄-haloalkyl;
R² represents hydrogen, C₁-C₄-alkyl or C₁-C₄-haloalkyl; or
R¹ and R² together with the carbon atom to which they are attached form a 3- to 6-membered cycloalkyl or heterocycloalkyl ring;
R³ represents phenyl, which is optionally substituted, one or more times, independently of each other, with R⁸;
R⁴ represents hydrogen, C₁-C₄-alkyl, C₁-C₄-haloalkyl or C₃-C₆-cycloalkyl;
R⁵ represents hydrogen or C₁-C₄-alkyl;
R⁶ represent hydrogen, C₁-C₆-alkyl, wherein said C₁-C₆-alkyl group is optionally substituted with R¹⁴, C₂-C₄-hydroxyalkyl, (C₁-C₄-alkoxy)-(C₂-C₄-alkyl)-, C₃-C₆-cycloalkyl, C₁-C₄-haloalkyl, C₃-C₆-halocycloalkyl, 3- to 6-membered heterocycloalkyl, heterospirocycloalkyl, phenyl, heteroaryl, heterocycloalkyl fused with phenyl or heteroaryl, 3- to 7-membered heterocycloalkyl-(C₁-C₃-alkyl)-, heterospirocycloalkyl-(C₁-C₃-alkyl)-, (heterocycloalkyl fused with phenyl or heteroaryl)-(C₁-C₃-alkyl)-, phenyl-(C₁-C₃-alkyl)- or heteroaryl-(C₁-C₃-alkyl)-, wherein said 3- to 6-membered or 3- to 7-membered heterocycloalkyl, heterospirocycloalkyl, heterocycloalkyl fused with phenyl or heteroaryl, phenyl or heteroaryl groups are optionally substituted, one or more times, independently of each other, with R⁹, or
R⁵ and R⁶ together with the nitrogen atom to which they are attached form a 4- to 7-membered nitrogen containing heterocyclic ring, optionally containing one additional heteroatom or heteroatom containing group selected from O, NH, S, and SO₂, and which may be optionally substituted, one or more times, independently of each other, with R⁹, or a 1,2,3,4-tetrahydroisoquinoline, or a 3-azabicyclo[3.1.0]hexane;
R^{7a} represents hydrogen or C₁-C₄-alkyl;
R^{7b} represents hydrogen or C₁-C₄-alkyl;
R⁸ represents halogen, cyano, C₁-C₄-alkyl, C₁-C₄-haloalkyl, C₁-C₃-alkoxy, C₁-C₃-haloalkoxy, C₃-C₆-cycloalkyl, C₃-C₆-cycloalkyl-(C₁-C₃-alkyl)-, R¹³—(C═O)—, R¹⁰—O—(C═O)—, R¹¹—NH—(C═O)—, or R¹²—(SOₓ)—;
R⁹ represents halogen, cyano, C₁-C₄-alkyl, C₁-C₄-haloalkyl, H₂N—C₁-C₄-alkyl, C₁-C₃-alkoxy, C₁-C₃-haloalkoxy, C₃-C₆-cycloalkyl, R¹⁵—(C═O)—, R¹⁰—O—(C═O)—, R^{11a}R^{11b}N—, R¹⁵—(C═O)—R^{11a}N—, R^{11a}R^{11b}N—(C═O)—, R¹²—(SOₓ)—; oxo, 5- to 6-membered heterocycloalkyl-, 5- to 6-membered heterocycloalkyl-(C₁-C₃-alkyl)-, benzyl, phenyl, or heteroaryl, wherein said phenyl or heteroaryl group is optionally substituted, one or more times, independently of each other, with halogen, C₁-C₄-alkyl, C₁-C₄-haloalkyl, C₁-C₃-alkoxy, or C₁-C₃-haloalkoxy;
R¹⁰ represents hydrogen, C₁-C₄-alkyl, or benzyl;
R^{11a} represents hydrogen or C₁-C₃-alkyl;
R^{11b} represents hydrogen, C₁-C₃-alkyl, C₃-C₆-cycloalkyl, methoxyethyl, or
R^{11a} and R^{11b} together with the nitrogen atom to which they are attached form a 5- to 6-membered nitrogen containing heterocyclic ring, optionally containing one additional heteroatom selected from O, NH, and S, and which may be optionally substituted, with (C₁-C₃-alkyl)-(C═O)—;
R¹² represents C₁-C₄-alkyl or phenyl;
R¹³ represents C₁-C₄-alkyl, C₁-C₄-haloalkyl, (C₁-C₄-alkoxy)-(C₁-C₄-alkyl)-, C₁-C₄-alkyl-(C═O)—, C₃-C₆-cycloalkyl, or phenyl, wherein said C₃-C₆-cycloalkyl group is optionally substituted with C₁-C₄-alkyl or hydroxy and said phenyl group is optionally substituted, one or more times, independently of each other, with halogen, C₁-C₄-alkyl, C₁-C₄-haloalkyl, C₁-C₃-alkoxy, or C₁-C₃-haloalkoxy;
R¹⁴ represents cyano, R^{11a}R^{11b}N—, R^{11a}R^{11b}N—(C═O)—, or R¹²—(SOₓ)—;
R¹⁵ represents C₁-C₃-alkyl or phenyl;
x represents 0, 1, or 2;
   and stereoisomers, tautomers, N-oxides, hydrates, solvates, and salts thereof, and mixtures of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means 1, 2, 3, 4 or 5, particularly 1, 2, 3 or 4, more particularly 1, 2 or 3, even more particularly 1 or 2.

As used herein, an oxo substituent represents an oxygen atom, which is bound to a carbon atom via a double bond.

Should a composite substituent be composed of more than one parts, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)-group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulphur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₄-alkyl” means a linear or branched, saturated, monovalent hydrocarbon group having 1, 2, 3, or 4 carbon atoms, e.g. a methyl, ethyl, propyl, isopropyl, butyl, sec-butyl, isobutyl, tert-butyl. Particularly, said group has 1, 2, or 3 carbon atoms (“C₁-C₃-alkyl”), e.g. a methyl, ethyl, propyl, or isopropyl group, more particularly 1 or 2 carbon atoms (“C₁-C₂-alkyl”), e.g. a methyl or ethyl group.

The term “C₂-C₄-hydroxyalkyl” means a linear or branched, saturated, monovalent hydrocarbon group in which the term “C₂-C₄-alkyl” is defined supra, and in which one hydrogen atom is replaced with a hydroxy group, e.g. a 1-hydroxyethyl, 2-hydroxyethyl, 3-hydroxypropyl, 2-hydroxypropyl, 1-hydroxypropyl, 1-hydroxypropan-2-yl, 2-hydroxypropan-2-yl, 3-hydroxy-2-methyl-propyl, 2-hydroxy-2-methyl-propyl, 1-hydroxy-2-methyl-propyl group.

The term “C₁-C₄-haloalkyl” means a linear or branched, saturated, monovalent hydrocarbon group in which the term “C₁-C₄-alkyl” is as defined supra, and in which one or more of the hydrogen atoms are replaced, identically or differently, with a halogen atom. Particularly, said halogen atom is a fluorine atom. Said C₁-C₄-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₄-alkoxy” means a linear or branched, saturated, monovalent group of formula (C₁-C₄-alkyl)-O—, in which the term “C₁-C₄-alkyl” is as defined supra, e.g. a methoxy, ethoxy, n-propoxy, isopropoxy, n-butoxy, sec-butoxy, isobutoxy, or tert-butoxy group.

The term “C₁-C₄-haloalkoxy” means a linear or branched, saturated, monovalent C₁-C₄-alkoxy group, as defined supra, in which one or more of the hydrogen atoms is replaced, identically or differently, with a halogen atom. Particularly, said halogen atom is a fluorine atom. Said C₁-C₄-haloalkoxy group is, for example, fluoromethoxy, difluoromethoxy, trifluoromethoxy, 2,2,2-trifluoroethoxy or pentafluoroethoxy.

The term “C₃-C₆-cycloalkyl” means a saturated, monovalent, monocyclic hydrocarbon ring which contains 3, 4, 5, or 6 carbon atoms (“C₃-C₆-cycloalkyl”). Said C₃-C₆-cycloalkyl group is for example, e.g. a cyclopropyl, cyclobutyl, cyclopentyl, or cyclohexyl group.

The term “C₃-C₆-halocycloalkyl” means a saturated, monovalent, monocyclic hydrocarbon ring in which the term “C₃-C₆-halocycloalkyl” is as defined supra, and in which one or more of the hydrogen atoms are replaced, identically or differently, with a halogen atom. Particularly, said halogen atom is a fluorine atom.

The term “4- to 7-membered heterocycloalkyl” means a monocyclic, saturated heterocycle with 4, 5, 6, or 7 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen or oxygen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen or oxygen atom and optionally one further ring heteroatom from the series: N, O.

The term “heterocycloalkyl fused with phenyl or heteroaryl” means a bicyclic heterocycle with 8, 9 or 10 ring atoms in total, in which the two rings share two adjacent ring atoms, and in which the “heterocycloalkyl” part contains one or two identical or different ring heteroatoms from the series: N, O and/or S, and the term “heteroaryl” means a monocyclic aromatic ring having 5 or 6 ring atoms (a “5- to 6-membered heteroaryl” group), which contains at least one ring heteroatom and optionally one, two or three further ring heteroatoms from the series N, O and/or S; it being possible for said fused heterocycloalkyl group to be attached to the rest of the molecule via any one of the carbon atoms or, if present, a nitrogen atom.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro [5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “heteroaryl” means a monovalent, monocyclic, bicyclic or tricyclic aromatic ring having 5, 6, 8, 9, or 10 ring atoms (a “5- to 10-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly, the heteroaryl group is a pyridinyl group.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄-C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons.

In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, 36Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D20 can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases, deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases, the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an indazole moiety can exist as a 1H tautomer, or a 2H tautomer, or even a mixture in any amount of the two tautomers, namely:

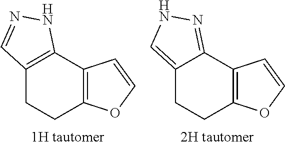

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive but are converted (for example metabolically or hydrolytically) into compounds according to the invention during their residence time in the body.

In accordance with a second embodiment of the first aspect, the present invention covers compounds of general formula (I), supra, in which:
R¹ represents hydrogen or C₁-C₃-alkyl;
R² represents hydrogen or C₁-C₃-alkyl;
R³ represents phenyl, which is optionally substituted, one or two times, independently of each other, with R⁸;
R⁴ represents hydrogen, C₁-C₃-alkyl;
R⁵ represents hydrogen or C₁-C₃-alkyl;
R⁶ represent C₁-C₆-alkyl, wherein said C₁-C₆-alkyl group is optionally substituted with R¹⁴, C₂-C₄-hydroxyalkyl, (C₁-C₃-alkoxy)-(C₂-C₃-alkyl)-, C₃-C₆-cycloalkyl, C₁-C₃-haloalkyl, 3- to 6-membered heterocycloalkyl, phenyl, heteroaryl, heterocycloalkyl fused with phenyl, 3- to 7-membered heterocycloalkyl-(C₁-C₃-alkyl)-, (heterocycloalkyl fused with phenyl)-(C₁-C₃-alkyl)-, phenyl-(C₁-C₃-alkyl)- or heteroaryl-(C₁-C₃-alkyl)-, wherein said 3- to 6-membered or 3- to 7-membered heterocycloalkyl, heterocycloalkyl fused with phenyl, phenyl or heteroaryl groups are optionally substituted, one or more times, independently of each other, with R⁹, or
R⁵ and R⁶ together with the nitrogen atom to which they are attached form a 4- to 7-membered nitrogen containing heterocyclic ring, optionally containing one additional heteroatom or heteroatom containing group selected from O, NH, S, and SO₂, and which may be optionally substituted, one or more times, independently of each other, with R⁹, or a 1,2,3,4-tetrahydroisoquinoline, or a 3-azabicyclo[3.1.0]hexane;
R^{7a} represents hydrogen or C₁-C₃-alkyl;
R^{7b} represents hydrogen or C₁-C₃-alkyl;
R⁸ represents halogen, C₁-C₃-alkyl;
R⁹ represents halogen, C₁-C₄-alkyl, C₁-C₃-haloalkyl, C₁-C₃-alkoxy, R¹⁵—(C═O)—, R¹⁰—O—(C═O)—, R^{11a}R^{11b}N—, R¹⁵—(C═O)—R^{11a}N—, R^{11a}R^{11b}N—(C═O)—, R¹²—(SOₓ)—; oxo, benzyl, phenyl, or heteroaryl, wherein said phenyl or heteroaryl group is optionally substituted, one or more times, independently of each other, with halogen, C₁-C₃-alkyl, or C₁-C₃-alkoxy;
R¹⁰ represents C₁-C₄-alkyl, or benzyl;
R^{11a} represents hydrogen or C₁-C₃-alkyl;
R^{11b} represents hydrogen, C₁-C₃-alkyl, C₃-C₆-cycloalkyl, methoxyethyl, or
R^{11a} and R^{11b} together with the nitrogen atom to which they are attached form a 5- to 6-membered nitrogen containing heterocyclic ring, optionally containing one additional heteroatom selected from O, NH, and S, and which may be optionally substituted, with (C₁-C₃-alkyl)-(C═O)—;
R¹² represents C₁-C₃-alkyl;
R¹⁴ represents cyano, R^{11a}R^{11b}N—, R^{11a}R^{11b}N—(C═O)—, or R¹²—(SOₓ)—;
R¹⁵ represents C₁-C₃-alkyl or phenyl;
x represents 0, 1, or 2;
   and stereoisomers, tautomers, N-oxides, hydrates, solvates, and salts thereof, and mixtures of same.

In accordance with a third embodiment of the first aspect, the present invention covers compounds of general formula (I), supra, in which:
R¹ represents hydrogen;
R² represents hydrogen;
R³ represents phenyl, which is optionally substituted, one or two times, independently of each other, with R⁸;
R⁴ represents methyl;
R⁵ represents hydrogen or methyl;
R⁶ represent C₁-C₅-alkyl, wherein said C₁-C₅-alkyl group is optionally substituted with R¹⁴, C₂-C₄-hydroxyalkyl, (C₁-C₃-alkoxy)-(C₂-C₃-alkyl)-, C₃-C₅-cycloalkyl, difluoroethyl, 6-membered heterocycloalkyl, phenyl, heterocycloalkyl fused with phenyl, 4- to 7-membered heterocycloalkyl-(C₁-C₃-alkyl)-, (heterocycloalkyl fused with phenyl)-methyl, phenyl-(C₁-C₃-alkyl)- or heteroaryl-(C₁-C₂-alkyl)-, wherein said 6-membered or 4- to 7-membered heterocycloalkyl, phenyl or heteroaryl groups are optionally substituted, one or more times, independently of each other, with R⁹, or
R⁵ and R⁶ together with the nitrogen atom to which they are attached form a 4- to 7-membered nitrogen containing heterocyclic ring, optionally containing one additional heteroatom or heteroatom containing group selected from O, NH, and SO₂, and which may be optionally substituted, one or two times, with R⁹, or a 1,2,3,4-tetrahydroisoquinoline, or a 3-azabicyclo[3.1.0]hexane;
R^{7a} represents hydrogen;
R^{7b} represents hydrogen or methyl;
R⁸ represents fluoro, chloro or methyl;
R⁹ represents fluoro, chloro, bromo, C₁-C₃-alkyl, trifluoromethyl, C₁-C₃-alkoxy, R¹⁵—(C═O)—, R¹⁰—O—(C═O)—, R^{11a}R^{11b}N—, CH₃—(C═O)—HN—, R^{11a}R^{11b}N—(C═O)—, R¹²—(SOₓ)—, oxo, benzyl, or phenyl, wherein said phenyl group is optionally substituted, one or two times, independently of each other, with fluoro, methyl, or methoxy;
R¹⁰ represents ethyl or tert.-butyl;
R^{11a} represents hydrogen, methyl or ethyl;
R^{11b} represents hydrogen, methyl, ethyl, cyclopropyl, cyclohexyl, or methoxyethyl, or
R^{11a} and R^{11b} together with the nitrogen atom to which they are attached form a 5- to 6-membered nitrogen containing heterocyclic ring, optionally containing one additional heteroatom selected from O, and NH, and which may be optionally substituted, with methyl-(C═O)—;
R¹² represents methyl;
R¹⁴ represents cyano, R^{11a}R^{11b}N—, R^{11a}R^{11b}N—(C═O)—, or R¹²—(SOₓ)—;
R¹⁵ represents methyl or phenyl;
x represents 0, 1, or 2;
   and stereoisomers, tautomers, N-oxides, hydrates, solvates, and salts thereof, and mixtures of same.

In accordance with a fourth embodiment of the first aspect, the present invention covers compounds of general formula (I), supra, in which:
R¹ represents hydrogen;
R² represents hydrogen;
R³ represents phenyl, which is optionally substituted, one or two times, independently of each other, with R⁸;
R⁴ represents methyl;
R⁵ represents hydrogen or methyl;
R⁶ represents methyl, —CH₂CH₂CH₃, cyclopropyl, —CH₂CH₂OH, cyclopropylmethyl, —CH₂CH₂CN, —CH₂CH₂OCH₃, —CH₂CH₂CH₂OH, —CH₂CNH₂O, —CH₂CHF₂, —CH₂CH₂CH(CH₃)₂, cyclopentyl, —CH₂CH₂N(CH₃)₂, —C(CH₃)₂CN, —CH₂C(CH₃)₂OH, 3-methoxypropyl, —CH₂CH₂CNH₂O, —CH₂CH₂SCH₃O, 2-(azetidin-1-yl)ethyl, (pyrrolidin-2-yl)methyl, (pyrrolidin-2-yl)methyl, 3-ethoxypropyl, oxan-4-yl, (tetrahydrofuran-2-yl)methyl, (11H-pyrazol-3-yl)methyl, (1H-imidazol-2-yl)methyl, (furan-2-yl)methyl, —CH₂CON(CH₃)₂, (ethylcarbamoyl)methyl, (1,2-oxazol-5-yl)methyl, (1,2-oxazol-3-yl)methyl, (thiophen-2-yl)methyl, (1,3-thiazol-2-yl)methyl, 4-methylphenyl, 2-methylphenyl, benzyl, 2-(diethylamino)ethyl, 2-(pyrrolidin-1-yl)ethyl, (pyridin-3-yl)methyl, (pyridin-4-yl)methyl, (pyridin-2-yl)methyl, 3-(propan-2-yloxy)propyl, (oxan-4-yl)methyl, (cyclopropylcarbamoyl)methyl, 6-oxopiperidin-3-yl, 2-fluorophenyl, 4-fluorophenyl, 3-fluorophenyl, (1,4-dioxan-2-yl)methyl, 2-(thiophen-2-yl)ethyl, 2-chlorophenyl, 2-bromophenyl, 2,6-dimethylphenyl, 2-phenylethyl, (2-methylphenyl)methyl, (4-methylphenyl)methyl, 3,4-dimethylphenyl, (3-methylphenyl)methyl, 2-ethylphenyl, 2,4-dimethylphenyl, 2,5-dimethylphenyl, 2,3-dimethylphenyl, 3,5-dimethylphenyl, 3-(diethylamino)propyl, 2-(piperidin-1-yl)ethyl, 2-methoxyphenyl, 4-methoxyphenyl, 3-methoxyphenyl, 2-(morpholin-4-yl)ethyl, (diethylcarbamoyl)methyl, 2-oxo-2-(pyrrolidin-1-yl)ethyl, 3-fluoro-4-methylphenyl, (4-fluorophenyl)methyl, 2,4-difluorophenyl, 2,5-difluorophenyl, 2-chloro-4-methylphenyl, 5-chloro-2-methylphenyl, 3-chloro-2-methylphenyl, (3-bromophenyl)methyl, 3-phenylpropyl, (4-ethylphenyl)methyl, 2,4,6-trimethylphenyl, 2-ethyl-6-methylphenyl, 4-(propan-2-yl)phenyl, 2-(azepan-1-yl)ethyl, 3-(piperidin-1-yl)propyl, (3-methoxyphenyl)methyl, 4-ethoxyphenyl, (2-methoxyphenyl)methyl, (4-methoxyphenyl)methyl, 3-acetylphenyl, 4-acetylphenyl, 3-(morpholin-4-yl)propyl, 3-(2-oxopyrrolidin-1-yl)propyl, 2H-1,3-benzodioxol-5-yl, 2-(morpholin-4-yl)-2-oxoethyl, 3-(azepan-1-yl)propyl, 2-(4-ethylpiperazin-1-yl)ethyl, (4-ethoxyphenyl)methyl, (2-ethoxyphenyl)methyl, (4-carbamoylphenyl)methyl, 4-acetamidophenyl, 3,5-dimethoxyphenyl, 2,5-dimethoxyphenyl, 2,4-dimethoxyphenyl, 3,4-dimethoxyphenyl, 3-(trifluoromethyl)phenyl, (4-acetamidophenyl)methyl, (2,3-dimethoxyphenyl)methyl, (2,5-dimethoxyphenyl)methyl, 4-(ethoxycarbonyl)phenyl, 2-benzoyl-4-chlorophenyl,

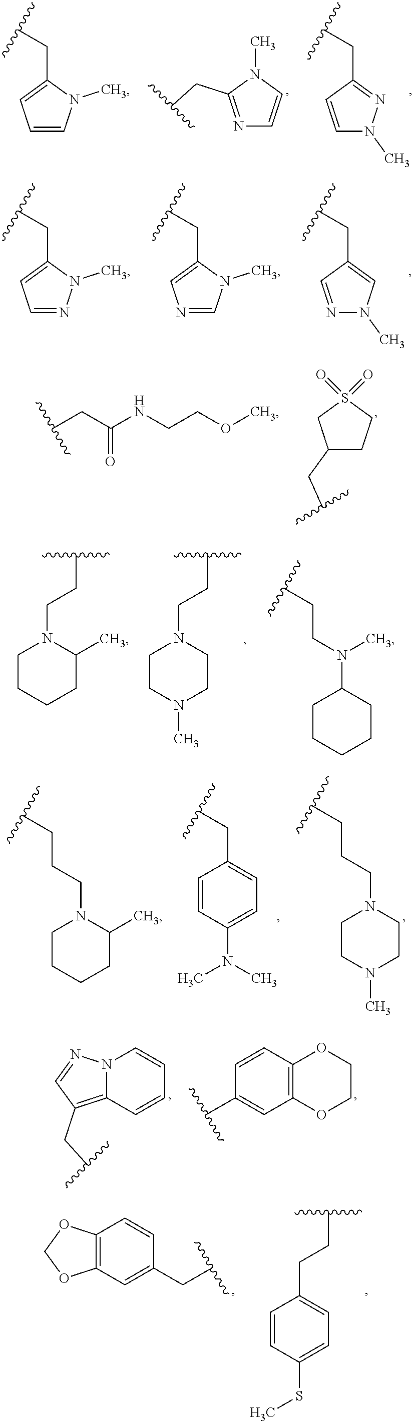

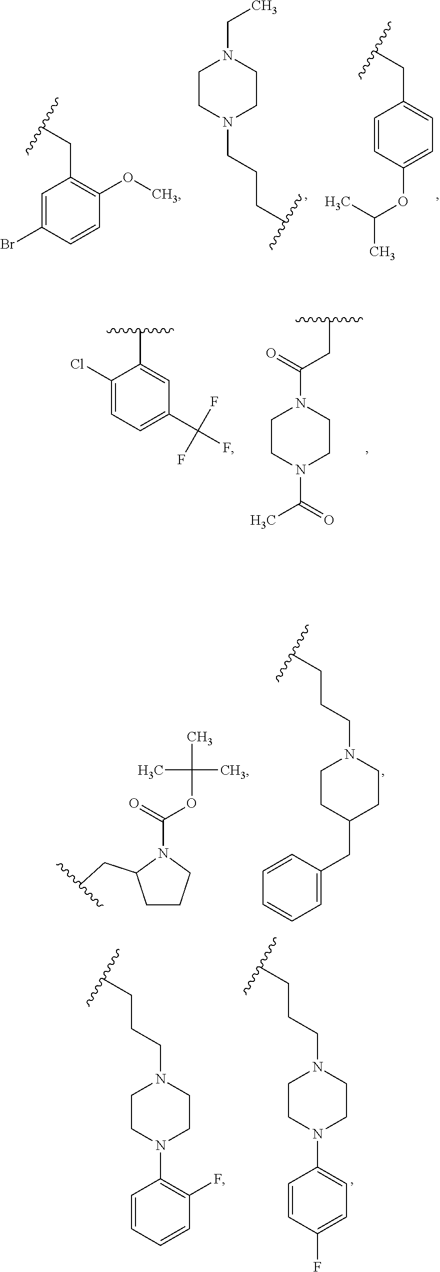

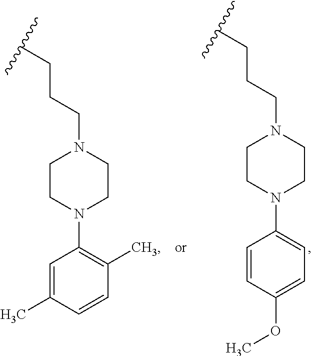 [SMILES: *CCCN1CCN(c2cc(C)ccc2C)CC1 ; *CCCN1CCN(c2ccc(OC)cc2)CC1]

or
R⁵ and R⁶ together with the nitrogen atom to which they are attached form a 4- to 7-membered nitrogen containing heterocyclic ring, optionally containing one additional heteroatom or heteroatom containing group selected from O, NH and SO₂, and which may be optionally substituted, one or two times, independently of each other, with R⁹, or a 1,2,3,4-tetrahydroisoquinoline, or a 3-azabicyclo[3.1.0]hexane;
R^{7a} represents hydrogen;
R^{7b} represents hydrogen or methyl;
R⁸ represents fluoro, chloro, or methyl;
R⁹ represents hydroxy, C₁-C₄-alkyl, C₃-C₆-cycloalkyl, HO—(C₁-C₃-alkyl)-, (C₁-C₃-alkyl)-(C═O)—, (C₁-C₃-alkyl)-O—(C═O)—, H₂N—(C═O)—, H₃C—SO₂—, phenyl, or benzyl;
   and stereoisomers, tautomers, N-oxides, hydrates, solvates, and salts thereof, and mixtures of same.

In accordance with a fifth embodiment of the first aspect, the present invention covers compounds of general formula (I), supra, in which:
R¹ represents hydrogen;
R² represents hydrogen;
R³ represents phenyl, which is substituted, one or two times, independently of each other, with R⁸;
R⁴ represents methyl;
R⁵ represents hydrogen;
R⁶ represents —CH₂CH₂OCH₃, —CH₂CH₂N(CH₃)₂, (pyrrolidin-2-yl)methyl, (pyrrolidin-2-yl)methyl, (tetrahydrofuran-2-yl)methyl, (1H-pyrazol-3-yl)methyl, (ethylcarbamoyl)methyl, (1,2-oxazol-3-yl)methyl, (1,3-thiazol-2-yl)methyl, 2-(pyrrolidin-1-yl)ethyl, (pyridin-2-yl)methyl, (1,4-dioxan-2-yl)methyl, (2-methylphenyl)methyl, (4-methylphenyl)methyl, (3-methylphenyl)methyl, 2-(piperidin-1-yl)ethyl, 2-(morpholin-4-yl)ethyl, (3-methoxyphenyl)methyl, (4-methoxyphenyl)methyl, 2-(4-ethylpiperazin-1-yl)ethyl, (4-carbamoylphenyl)methyl, (4-acetamidophenyl)methyl,

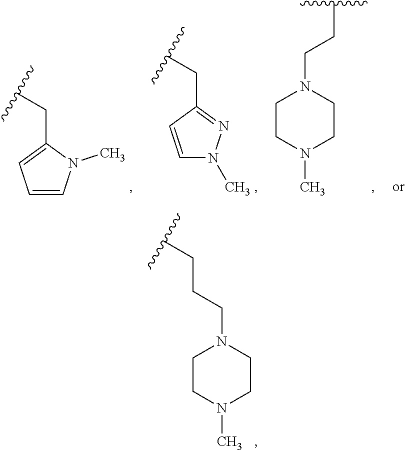

or
R⁵ and R⁶ together with the nitrogen atom to which they are attached form a 3- to 6-membered nitrogen containing heterocyclic ring, optionally containing one additional heteroatom or heteroatom containing group selected from O, NH and S, and which may be optionally substituted, one or more times, independently of each other, with R⁹;
R^{7a} represents hydrogen;
R^{7b} represents hydrogen or methyl;
R^{a} represents fluoro, chloro, or methyl;
R⁹ represents;
   and stereoisomers, tautomers, N-oxides, hydrates, solvates, and salts thereof, and mixtures of same.

In accordance with a sixth embodiment of the first aspect, the present invention covers compounds of general formula (I), supra, which are selected from the group consisting of:
N-[(2R)-1,4-dioxan-2-ylmethyl]-8-methyl-2-(4-methylbenzyl)-4,5-dihydro-2H-furo[2,3-g]indazole-7-carboxamide,
N-[(2R)-1,4-dioxan-2-ylmethyl]-8-methyl-2-(2-methylbenzyl)-4,5-dihydro-2H-furo[2,3-g]indazole-7-carboxamide,
2-(2-chlorobenzyl)-8-methyl-N-[(2R*)-tetrahydrofuran-2-ylmethyl]-4,5-dihydro-2H-furo[2,3-g]indazole-7-carboxamide,
8-methyl-2-(4-methylbenzyl)-N-[(2S)-tetrahydrofuran-2-ylmethyl]-4,5-dihydro-2H-furo[2,3-g]indazole-7-carboxamide,
8-methyl-2-(2-methylbenzyl)-N-[(2S)-tetrahydrofuran-2-ylmethyl]-4,5-dihydro-2H-furo[2,3-g]indazole-7-carboxamide,
2-(2-chlorobenzyl)-8-methyl-N-(4-methylbenzyl)-4,5-dihydro-2H-furo[2,3-g]indazole-7-carboxamide,
2-(2-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-[(2RS)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
8-methyl-2-(4-methylbenzyl)-N-(1,2-oxazol-3-ylmethyl)-4,5-dihydro-2H-furo[2,3-g]indazole-7-carboxamide,
2-(4-chlorobenzyl)-8-methyl-N-(1,2-oxazol-3-ylmethyl)-4,5-dihydro-2H-furo[2,3-g]indazole-7-carboxamide,
2-(2-chloro-4-fluorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-[(1-methyl-1H-pyrazol-3-yl)methyl]-4,5-dihydro-2H-furo[2,3-g]indazole-7-carboxamide,
2-(4-chlorobenzyl)-8-methyl-N-(1,3-thiazol-2-ylmethyl)-4,5-dihydro-2H-furo[2,3-g]indazole-7-carboxamide,
8-methyl-2-(2-methylbenzyl)-N-(1,2-oxazol-3-ylmethyl)-4,5-dihydro-2H-furo[2,3-g]indazole-7-carboxamide,
2-(2-chloro-4-fluorobenzyl)-8-methyl-N-(1,2-oxazol-3-ylmethyl)-4,5-dihydro-2H-furo[2,3-g]indazole-7-carboxamide,
2-(2-chlorobenzyl)-8-methyl-N-[2-(pyrrolidin-1-yl)ethyl]-4,5-dihydro-2H-furo[2,3-g]indazole-7-carboxamide,
2-(4-chlorobenzyl)-8-methyl-N-[2-(4-methylpiperazin-1-yl)ethyl]-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
2-(2-chlorobenzyl)-8-methyl-N-[2-(piperidin-1-yl)ethyl]-4,5-dihydro-2H-furo[2,3-g]indazole-7-carboxamide,
2-(4-chlorobenzyl)-N-[2-(ethylamino)-2-oxoethyl]-8-methyl-4,5-dihydro-2H-furo[2,3-g]indazole-7-carboxamide,
2-(4-chlorobenzyl)-8-methyl-N-(1H-pyrazol-3-ylmethyl)-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
8-methyl-2-(2-methylbenzyl)-N-[(2RS)-pyrrolidin-2-ylmethyl]-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
N-(4-carbamoylbenzyl)-2-(4-chlorobenzyl)-8-methyl-4,5-dihydro-2H-furo[2,3-g]indazole-7-carboxamide,
2-[(1RS)-1-(2-fluorophenyl)ethyl]-8-methyl-N-[(2S)-tetrahydrofuran-2-ylmethyl]-4,5-dihydro-2H-furo[2,3-g]indazole-7-carboxamide,
2-(3-chlorobenzyl)-8-methyl-N-[2-(piperidin-1-yl)ethyl]-4,5-dihydro-2H-furo[2,3-g]indazole-7-carboxamide,
N-(4-acetamidobenzyl)-2-(4-chlorobenzyl)-8-methyl-4,5-dihydro-2H-furo[2,3-g]indazole-7-carboxamide,
2-(2-chloro-4-fluorobenzyl)-8-methyl-N-[2-(4-methylpiperazin-1-yl)ethyl]-4,5-dihydro-2H-furo[2,3-g]indazole-7-carboxamide,
2-(4-chlorobenzyl)-8-methyl-N-[(1-methyl-1H-pyrrol-2-yl)methyl]-4,5-dihydro-2H-furo[2,3-g]indazole-7-carboxamide,
2-(3-chlorobenzyl)-8-methyl-N-[2-(pyrrolidin-1-yl)ethyl]-4,5-dihydro-2H-furo[2,3-g]indazole-7-carboxamide,
2-(4-chlorobenzyl)-8-methyl-N-(1,2-oxazol-5-ylmethyl)-4,5-dihydro-2H-furo[2,3-g]indazole-7-carboxamide,
2-(4-chlorobenzyl)-8-methyl-N-[2-(pyrrolidin-1-yl)ethyl]-4,5-dihydro-2H-furo[2,3-g]indazole-7-carboxamide,
2-(4-chlorobenzyl)-8-methyl-N-[2-oxo-2-(pyrrolidin-1-yl)ethyl]-4,5-dihydro-2H-furo[2,3-g]indazole-7-carboxamide,
N-[2-(azetidin-1-yl)ethyl]-2-(2-chlorobenzyl)-8-methyl-4,5-dihydro-2H-furo[2,3-g]indazole-7-carboxamide,
8-methyl-2-(2-methylbenzyl)-N-[(2RS)-pyrrolidin-2-ylmethyl]-4,5-dihydro-2H-furo[2,3-g]indazole-7-carboxamide hydrochloride (1:1),
2-(4-chlorobenzyl)-N-[2-(dimethylamino)-2-oxoethyl]-8-methyl-4,5-dihydro-2H-furo[2,3-g]indazole-7-carboxamide,
2-(4-chlorobenzyl)-8-methyl-N-[2-(piperidin-1-yl)ethyl]-4,5-dihydro-2H-furo[2,3-g]indazole-7-carboxamide,
2-(4-chlorobenzyl)-8-methyl-N-(pyridin-4-ylmethyl)-4,5-dihydro-2H-furo[2,3-g]indazole-7-carboxamide,
N-[2-(azetidin-1-yl)ethyl]-2-(3-chlorobenzyl)-8-methyl-4,5-dihydro-2H-furo[2,3-g]indazole-7-carboxamide,
N-[2-(azetidin-1-yl)ethyl]-2-(4-chlorobenzyl)-8-methyl-4,5-dihydro-2H-furo[2,3-g]indazole-7-carboxamide,
2-(4-chlorobenzyl)-8-methyl-N-[(1-methyl-1H-pyrazol-5-yl)methyl]-4,5-dihydro-2H-furo[2,3-g]indazole-7-carboxamide,
2-(4-chlorobenzyl)-8-methyl-N-[(1-methyl-1H-pyrazol-4-yl)methyl]-4,5-dihydro-2H-furo[2,3-g]indazole-7-carboxamide,
N-(2-amino-2-oxoethyl)-2-(4-chlorobenzyl)-8-methyl-4,5-dihydro-2H-furo[2,3-g]indazole-7-carboxamide,
tert-butyl (2RS)-2-[({[8-methyl-2-(2-methylbenzyl)-4,5-dihydro-2H-furo[2,3-g]indazol-7-yl]carbonyl}amino)methyl]pyrrolidine-1-carboxylate,
2-(4-chlorobenzyl)-N-{2-[(2-methoxyethyl)amino]-2-oxoethyl}-8-methyl-4,5-dihydro-2H-furo[2,3-g]indazole-7-carboxamide,
2-(4-chlorobenzyl)-N-[2-(diethylamino)-2-oxoethyl]-8-methyl-4,5-dihydro-2H-furo[2,3-g]indazole-7-carboxamide,
(3-benzylazetidin-1-yl)[2-(4-chlorobenzyl)-8-methyl-4,5-dihydro-2H-furo[2,3-g]indazol-7-yl]methanone,
2-(4-chlorobenzyl)-8-methyl-N-(pyrazolo[1,5-a]pyridin-3-ylmethyl)-4,5-dihydro-2H-furo[2,3-g]indazole-7-carboxamide,
2-(4-chlorobenzyl)-N-(cyclopropylmethyl)-8-methyl-4,5-dihydro-2H-furo[2,3-g]indazole-7-carboxamide,
2-(4-chlorobenzyl)-8-methyl-N-(tetrahydro-2H-pyran-4-ylmethyl)-4,5-dihydro-2H-furo[2,3-g]indazole-7-carboxamide,
2-(4-chlorobenzyl)-8-methyl-N-[2-(morpholin-4-yl)-2-oxoethyl]-4,5-dihydro-2H-furo[2,3-g]indazole-7-carboxamide,
2-(4-chlorobenzyl)-8-methyl-N-[(1-methyl-1H-imidazol-2-yl)methyl]-4,5-dihydro-2H-furo[2,3-g]indazole-7-carboxamide,
2-(4-chlorobenzyl)-N-[2-(cyclopropylamino)-2-oxoethyl]-8-methyl-4,5-dihydro-2H-furo[2,3-g]indazole-7-carboxamide,
2-(4-chlorobenzyl)-8-methyl-N-[(1-methyl-1H-imidazol-5-yl)methyl]-4,5-dihydro-2H-furo[2,3-g]indazole-7-carboxamide,
2-(4-chlorobenzyl)-8-methyl-N-[2-(morpholin-4-yl)ethyl]-4,5-dihydro-2H-furo[2,3-g]indazole-7-carboxamide,
N-(3-amino-3-oxopropyl)-2-(4-chlorobenzyl)-8-methyl-4,5-dihydro-2H-furo[2,3-g]indazole-7-carboxamide,
2-(4-chlorobenzyl)-8-methyl-N-(2-phenylethyl)-4,5-dihydro-2H-furo[2,3-g]indazole-7-carboxamide,
2-(4-chlorobenzyl)-N-(2-hydroxyethyl)-8-methyl-4,5-dihydro-2H-furo[2,3-g]indazole-7-carboxamide,
2-(4-chlorobenzyl)-N-[(1,1-dioxidotetrahydrothiophen-3-yl)methyl]-8-methyl-4,5-dihydro-2H-furo[2,3-g]indazole-7-carboxamide,
2-(4-chlorobenzyl)-N-(2-hydroxy-2-methylpropyl)-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](3,4-dihydroisoquinolin-2(1H)-yl)methanone,
1-(4-{[2-(4-chlorobenzyl)-8-methyl-4,5-dihydro-2H-furo[2,3-g]indazol-7-yl]carbonyl}piperazin-1-yl)ethanone,
2-(4-chlorobenzyl)-8-methyl-N-[2-(methylsulfinyl)ethyl]-4,5-dihydro-2H-furo[2,3-g]indazole-7-carboxamide,
2-(4-chlorobenzyl)-N-(3-hydroxypropyl)-8-methyl-4,5-dihydro-2H-furo[2,3-g]indazole-7-carboxamide,
2-(4-chlorobenzyl)-N-(2,2-difluoroethyl)-8-methyl-4,5-dihydro-2H-furo[2,3-g]indazole-7-carboxamide,
2-(4-chlorobenzyl)-N,8-dimethyl-N-(pyridin-4-ylmethyl)-4,5-dihydro-2H-furo[2,3-g]indazole-7-carboxamide,
[2-(4-chlorobenzyl)-8-methyl-4,5-dihydro-2H-furo[2,3-g]indazol-7-yl][4-(methylsulfonyl)piperazin-1-yl]methanone,
3-azabicyclo[3.1.0]hexan-3-yl[2-(4-chlorobenzyl)-8-methyl-4,5-dihydro-2H-furo[2,3-g]indazol-7-yl]methanone,
N-[2-(4-acetylpiperazin-1-yl)-2-oxoethyl]-2-(4-chlorobenzyl)-8-methyl-4,5-dihydro-2H-furo[2,3-g]indazole-7-carboxamide,
2-(4-chlorobenzyl)-N,8-dimethyl-N-(pyridin-3-ylmethyl)-4,5-dihydro-2H-furo[2,3-g]indazole-7-carboxamide,
2-(4-chlorobenzyl)-N-(2-cyanoethyl)-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](4-hydroxypiperidin-1-yl)methanone,
2-(4-chlorobenzyl)-N-(2-furylmethyl)-N,8-dimethyl-4,5-dihydro-2H-furo[2,3-g]indazole-7-carboxamide,
2-(4-chlorobenzyl)-N,8-dimethyl-N-[(1-methyl-1H-pyrazol-3-yl)methyl]-4,5-dihydro-2H-furo[2,3-g]indazole-7-carboxamide,
2-(4-chlorobenzyl)-N-(1H-imidazol-2-ylmethyl)-N,8-dimethyl-4,5-dihydro-2H-furo[2,3-g]indazole-7-carboxamide,
[2-(4-chlorobenzyl)-8-methyl-4,5-dihydro-2H-furo[2,3-g]indazol-7-yl](2,6-dimethylmorpholin-4-yl)methanone,
2-(4-chlorobenzyl)-N,8-dimethyl-N-[(1-methyl-1H-pyrazol-4-yl)methyl]-4,5-dihydro-2H-furo[2,3-g]indazole-7-carboxamide,
2-(4-chlorobenzyl)-8-methyl-N-(tetrahydro-2H-pyran-4-yl)-4,5-dihydro-2H-furo[2,3-g]indazole-7-carboxamide,
2-(4-chlorobenzyl)-N-(1H-imidazol-2-ylmethyl)-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl][4-(hydroxymethyl)piperidin-1-yl]methanone,
2-(4-chlorobenzyl)-N-(2-methoxyethyl)-N,8-dimethyl-4,5-dihydro-2H-furo[2,3-g]indazole-7-carboxamide,
2-(4-chlorobenzyl)-8-methyl-N-(6-oxopiperidin-3-yl)-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}-D-prolinamide,
1-{[2-(4-chlorobenzyl)-8-methyl-4,5-dihydro-2H-furo[2,3-g]indazol-7-yl]carbonyl}piperidine-4-carboxamide,
2-(4-chlorobenzyl)-N,8-dimethyl-N-[(1-methyl-1H-pyrazol-5-yl)methyl]-4,5-dihydro-2H-furo[2,3-g]indazole-7-carboxamide,
2-(4-chlorobenzyl)-N-(2-hydroxyethyl)-N,8-dimethyl-4,5-dihydro-2H-furo[2,3-g]indazole-7-carboxamide,
2-(4-chlorobenzyl)-N-(2-cyanopropan-2-yl)-8-methyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}piperidine-3-carboxamide,
2-(4-chlorobenzyl)-N,8-dimethyl-N-(pyridin-2-ylmethyl)-4,5-dihydro-2H-furo[2,3-g]indazole-7-carboxamide,
[2-(4-chlorobenzyl)-8-methyl-4,5-dihydro-2H-furo[2,3-g]indazol-7-yl](1,1-dioxidothiomorpholin-4-yl)methanone,
1-{[2-(4-chlorobenzyl)-8-methyl-4,5-dihydro-2H-furo[2,3-g]indazol-7-yl]carbonyl}-L-prolinamide,
2-(2-chlorobenzyl)-N-[2-(diethylamino)ethyl]-8-methyl-4,5-dihydro-2H-furo[2,3-g]indazole-7-carboxamide,
2-(3-chlorobenzyl)-N-(2-ethoxybenzyl)-8-methyl-4,5-dihydro-2H-furo[2,3-g]indazole-7-carboxamide,
[2-(3-chlorobenzyl)-8-methyl-4,5-dihydro-2H-furo[2,3-g]indazol-7-yl](4-cyclohexylpiperazin-1-yl)methanone,
2-(3-chlorobenzyl)-8-methyl-N-[3-(4-methylpiperazin-1-yl)propyl]-4,5-dihydro-2H-furo[2,3-g]indazole-7-carboxamide,
2-(3-chlorobenzyl)-N-[3-(4-ethylpiperazin-1-yl)propyl]-8-methyl-4,5-dihydro-2H-furo[2,3-g]indazole-7-carboxamide,
2-(3-chlorobenzyl)-N-[2-(4-ethylpiperazin-1-yl)ethyl]-8-methyl-4,5-dihydro-2H-furo[2,3-g]indazole-7-carboxamide,
2-(3-chlorobenzyl)-N-(2,3-dimethoxybenzyl)-8-methyl-4,5-dihydro-2H-furo[2,3-g]indazole-7-carboxamide,
2-(3-chlorobenzyl)-8-methyl-N-[2-(4-methylpiperazin-1-yl)ethyl]-4,5-dihydro-2H-furo[2,3-g]indazole-7-carboxamide,
2-(3-chlorobenzyl)-N-(4-ethoxybenzyl)-8-methyl-4,5-dihydro-2H-furo[2,3-g]indazole-7-carboxamide,
2-(3-chlorobenzyl)-8-methyl-N-(2-methylbenzyl)-4,5-dihydro-2H-furo[2,3-g]indazole-7-carboxamide,
[2-(3-chlorobenzyl)-8-methyl-4,5-dihydro-2H-furo[2,3-g]indazol-7-yl](morpholin-4-yl)methanone,
2-(3-chlorobenzyl)-N-(2,5-dimethoxybenzyl)-8-methyl-4,5-dihydro-2H-furo[2,3-g]indazole-7-carboxamide,
2-(2-chlorobenzyl)-N-[2-(dimethylamino)ethyl]-8-methyl-4,5-dihydro-2H-furo[2,3-g]indazole-7-carboxamide,
2-(2-chlorobenzyl)-8-methyl-N-(3-methylbenzyl)-4,5-dihydro-2H-furo[2,3-g]indazole-7-carboxamide,
2-(2-chlorobenzyl)-N-cyclopentyl-8-methyl-4,5-dihydro-2H-furo[2,3-g]indazole-7-carboxamide,
2-(2-chlorobenzyl)-N-(4-methoxybenzyl)-8-methyl-4,5-dihydro-2H-furo[2,3-g]indazole-7-carboxamide,
2-(2-chlorobenzyl)-8-methyl-N-(2-phenylethyl)-4,5-dihydro-2H-furo[2,3-g]indazole-7-carboxamide,
2-(2-chlorobenzyl)-8-methyl-N-[(2RS)-tetrahydrofuran-2-ylmethyl]-4,5-dihydro-2H-furo[2,3-g]indazole-7-carboxamide,
2-(3-chlorobenzyl)-N-(2-methoxybenzyl)-8-methyl-4,5-dihydro-2H-furo[2,3-g]indazole-7-carboxamide,
2-(3-chlorobenzyl)-8-methyl-N-[3-(2-oxopyrrolidin-1-yl)propyl]-4,5-dihydro-2H-furo[2,3-g]indazole-7-carboxamide,
2-(3-chlorobenzyl)-8-methyl-N-(3-methylbenzyl)-4,5-dihydro-2H-furo[2,3-g]indazole-7-carboxamide,
2-(3-chlorobenzyl)-N-(3-isopropoxypropyl)-8-methyl-4,5-dihydro-2H-furo[2,3-g]indazole-7-carboxamide,
2-(4-chlorobenzyl)-8-methyl-N-[3-(morpholin-4-yl)propyl]-4,5-dihydro-2H-furo[2,3-g]indazole-7-carboxamide,
2-(4-chlorobenzyl)-8-methyl-N-(3-methylbenzyl)-4,5-dihydro-2H-furo[2,3-g]indazole-7-carboxamide,
2-(4-chlorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-N-(2-methoxybenzyl)-8-methyl-4,5-dihydro-2H-furo[2,3-g]indazole-7-carboxamide,
2-(4-chlorobenzyl)-8-methyl-N-(2-methylbenzyl)-4,5-dihydro-2H-furo[2,3-g]indazole-7-carboxamide,
[2-(4-chlorobenzyl)-8-methyl-4,5-dihydro-2H-furo[2,3-g]indazol-7-yl](morpholin-4-yl)methanone,
2-(4-chlorobenzyl)-8-methyl-N-[3-(piperidin-1-yl)propyl]-4,5-dihydro-2H-furo[2,3-g]indazole-7-carboxamide,
2-(4-chlorobenzyl)-N-[3-(4-ethylpiperazin-1-yl)propyl]-8-methyl-4,5-dihydro-2H-furo[2,3-g]indazole-7-carboxamide,
2-(2-chlorobenzyl)-8-methyl-N-[3-(2-oxopyrrolidin-1-yl)propyl]-4,5-dihydro-2H-furo[2,3-g]indazole-7-carboxamide,
[2-(4-chlorobenzyl)-8-methyl-4,5-dihydro-2H-furo[2,3-g]indazol-7-yl](4-cyclohexylpiperazin-1-yl)methanone,
2-(3-chlorobenzyl)-8-methyl-N-(pyridin-2-ylmethyl)-4,5-dihydro-2H-furo[2,3-g]indazole-7-carboxamide,
2-(3-chlorobenzyl)-N-[2-(dimethylamino)ethyl]-8-methyl-4,5-dihydro-2H-furo[2,3-g]indazole-7-carboxamide,
2-(3-chlorobenzyl)-N-(4-methoxybenzyl)-8-methyl-4,5-dihydro-2H-furo[2,3-g]indazole-7-carboxamide,
2-(3-chlorobenzyl)-8-methyl-N-(4-methylbenzyl)-4,5-dihydro-2H-furo[2,3-g]indazole-7-carboxamide,
2-(3-chlorobenzyl)-8-methyl-N-(3-methylbutyl)-4,5-dihydro-2H-furo[2,3-g]indazole-7-carboxamide,
2-(3-chlorobenzyl)-N-(3-methoxybenzyl)-8-methyl-4,5-dihydro-2H-furo[2,3-g]indazole-7-carboxamide,
2-(3-chlorobenzyl)-N-(3-ethoxypropyl)-8-methyl-4,5-dihydro-2H-furo[2,3-g]indazole-7-carboxamide,
2-(3-chlorobenzyl)-N-(3-methoxypropyl)-8-methyl-4,5-dihydro-2H-furo[2,3-g]indazole-7-carboxamide,
2-(3-chlorobenzyl)-N-cyclopropyl-8-methyl-4,5-dihydro-2H-furo[2,3-g]indazole-7-carboxamide,
2-(4-chlorobenzyl)-N-(4-methoxybenzyl)-8-methyl-4,5-dihydro-2H-furo[2,3-g]indazole-7-carboxamide,
2-(2-chlorobenzyl)-N-(3-ethoxypropyl)-8-methyl-4,5-dihydro-2H-furo[2,3-g]indazole-7-carboxamide,
2-(2-chlorobenzyl)-N-[3-(4-ethylpiperazin-1-yl)propyl]-8-methyl-4,5-dihydro-2H-furo[2,3-g]indazole-7-carboxamide,
[2-(3-chlorobenzyl)-8-methyl-4,5-dihydro-2H-furo[2,3-g]indazol-7-yl](pyrrolidin-1-yl)methanone,
N-(3,4-dimethylphenyl)-8-methyl-2-(4-methylbenzyl)-4,5-dihydro-2H-furo[2,3-g]indazole-7-carboxamide,
2-(2-chlorobenzyl)-8-methyl-N-(3-methylbutyl)-4,5-dihydro-2H-furo[2,3-g]indazole-7-carboxamide,
2-(4-chlorobenzyl)-N-(3-methoxypropyl)-8-methyl-4,5-dihydro-2H-furo[2,3-g]indazole-7-carboxamide,
8-methyl-2-(4-methylbenzyl)-N-(2-methylphenyl)-4,5-dihydro-2H-furo[2,3-g]indazole-7-carboxamide,
2-benzyl-N-(2,6-dimethylphenyl)-8-methyl-4,5-dihydro-2H-furo[2,3-g]indazole-7-carboxamide,
2-(4-chlorobenzyl)-N-(2-furylmethyl)-8-methyl-4,5-dihydro-2H-furo[2,3-g]indazole-7-carboxamide, N-benzyl-2-(4-chlorobenzyl)-8-methyl-4,5-dihydro-2H-furo[2,3-g]indazole-7-carboxamide,
8-methyl-2-(4-methylbenzyl)-N-(pyridin-3-ylmethyl)-4,5-dihydro-2H-furo[2,3-g]indazole-7-carboxamide,
2-(2-chlorobenzyl)-8-methyl-N-propyl-4,5-dihydro-2H-furo[2,3-g]indazole-7-carboxamide,
[2-(4-chlorobenzyl)-8-methyl-4,5-dihydro-2H-furo[2,3-g]indazol-7-yl](piperidin-1-yl)methanone,
8-methyl-N-(2-methylbenzyl)-2-(4-methylbenzyl)-4,5-dihydro-2H-furo[2,3-g]indazole-7-carboxamide,
2-(4-chlorobenzyl)-N-(2-methoxyethyl)-8-methyl-4,5-dihydro-2H-furo[2,3-g]indazole-7-carboxamide,
2-(3-chlorobenzyl)-8-methyl-N-propyl-4,5-dihydro-2H-furo[2,3-g]indazole-7-carboxamide,
2-(3-chlorobenzyl)-N,8-dimethyl-4,5-dihydro-2H-furo[2,3-g]indazole-7-carboxamide,
2-(4-chlorobenzyl)-N,8-dimethyl-4,5-dihydro-2H-furo[2,3-g]indazole-7-carboxamide,
2-(4-chlorobenzyl)-8-methyl-N-(pyridin-2-ylmethyl)-4,5-dihydro-2H-furo[2,3-g]indazole-7-carboxamide,
[2-(2-chlorobenzyl)-8-methyl-4,5-dihydro-2H-furo[2,3-g]indazol-7-yl](pyrrolidin-1-yl)methanone,
2-(4-chlorobenzyl)-8-methyl-N-(tetrahydrofuran-2-ylmethyl)-4,5-dihydro-2H-furo[2,3-g]indazole-7-carboxamide,
2-(4-chlorobenzyl)-8-methyl-N-(4-methylphenyl)-4,5-dihydro-2H-furo[2,3-g]indazole-7-carboxamide, N-benzyl-2-(2-chlorobenzyl)-8-methyl-4,5-dihydro-2H-furo[2,3-g]indazole-7-carboxamide,
2-(2-chlorobenzyl)-8-methyl-N-[2-(4-methylpiperazin-1-yl)ethyl]-4,5-dihydro-2H-furo[2,3-g]indazole-7-carboxamide,
2-(2-chlorobenzyl)-N-{2-[cyclohexyl(methyl)amino]ethyl}-8-methyl-4,5-dihydro-2H-furo[2,3-g]indazole-7-carboxamide,
[2-(2-chlorobenzyl)-8-methyl-4,5-dihydro-2H-furo[2,3-g]indazol-7-yl](4-propylpiperazin-1-yl)methanone,
N-(3-bromobenzyl)-8-methyl-2-(4-methylbenzyl)-4,5-dihydro-2H-furo[2,3-g]indazole-7-carboxamide,
N-(2-chlorophenyl)-8-methyl-2-(4-methylbenzyl)-4,5-dihydro-2H-furo[2,3-g]indazole-7-carboxamide,
N-(2-bromophenyl)-8-methyl-2-(4-methylbenzyl)-4,5-dihydro-2H-furo[2,3-g]indazole-7-carboxamide,
2-(2-chlorobenzyl)-8-methyl-N-(tetrahydrofuran-2-ylmethyl)-4,5-dihydro-2H-furo[2,3-g]indazole-7-carboxamide,
2-(4-chlorobenzyl)-N-(4-methoxyphenyl)-8-methyl-4,5-dihydro-2H-furo[2,3-g]indazole-7-carboxamide,
2-(2-chlorobenzyl)-8-methyl-N-[2-(morpholin-4-yl)ethyl]-4,5-dihydro-2H-furo[2,3-g]indazole-7-carboxamide,
2-(2-chlorobenzyl)-N-(3-methoxypropyl)-8-methyl-4,5-dihydro-2H-furo[2,3-g]indazole-7-carboxamide,
2-(4-chlorobenzyl)-N-(3-isopropoxypropyl)-8-methyl-4,5-dihydro-2H-furo[2,3-g]indazole-7-carboxamide,
2-(4-chlorobenzyl)-8-methyl-N-(2-thienylmethyl)-4,5-dihydro-2H-furo[2,3-g]indazole-7-carboxamide,
N-(2,5-dimethylphenyl)-8-methyl-2-(4-methylbenzyl)-4,5-dihydro-2H-furo[2,3-g]indazole-7-carboxamide,
N-(4-acetylphenyl)-8-methyl-2-(4-methylbenzyl)-4,5-dihydro-2H-furo[2,3-g]indazole-7-carboxamide,
2-(2-chlorobenzyl)-N-(2-methoxyethyl)-8-methyl-4,5-dihydro-2H-furo[2,3-g]indazole-7-carboxamide,
2-(4-chlorobenzyl)-8-methyl-N-(4-methylbenzyl)-4,5-dihydro-2H-furo[2,3-g]indazole-7-carboxamide,
N-(4-fluorophenyl)-8-methyl-2-(4-methylbenzyl)-4,5-dihydro-2H-furo[2,3-g]indazole-7-carboxamide,
azepan-1-yl[2-(3-chlorobenzyl)-8-methyl-4,5-dihydro-2H-furo[2,3-g]indazol-7-yl]methanone,
2-(4-chlorobenzyl)-N-(4-ethylbenzyl)-8-methyl-4,5-dihydro-2H-furo[2,3-g]indazole-7-carboxamide,
1-{[2-(3-chlorobenzyl)-8-methyl-4,5-dihydro-2H-furo[2,3-g]indazol-7-yl]carbonyl}piperidine-4-carboxamide,
2-(4-chlorobenzyl)-N-[2-(dimethylamino)ethyl]-8-methyl-4,5-dihydro-2H-furo[2,3-g]indazole-7-carboxamide,
2-(4-chlorobenzyl)-N-[3-(diethylamino)propyl]-8-methyl-4,5-dihydro-2H-furo[2,3-g]indazole-7-carboxamide,
2-(3-chlorobenzyl)-N-(2,3-dimethylphenyl)-8-methyl-4,5-dihydro-2H-furo[2,3-g]indazole-7-carboxamide,
2-(2-chlorobenzyl)-N-(2,6-dimethylphenyl)-8-methyl-4,5-dihydro-2H-furo[2,3-g]indazole-7-carboxamide,
2-(3-chlorobenzyl)-N-[4-(dimethylamino)benzyl]-8-methyl-4,5-dihydro-2H-furo[2,3-g]indazole-7-carboxamide,
2-(2-chlorobenzyl)-N-(3,4-dimethylphenyl)-8-methyl-4,5-dihydro-2H-furo[2,3-g]indazole-7-carboxamide,
N-(4-acetamidophenyl)-2-(2-chlorobenzyl)-8-methyl-4,5-dihydro-2H-furo[2,3-g]indazole-7-carboxamide,
2-(2-chloro-6-fluorobenzyl)-N-(3-fluorophenyl)-8-methyl-4,5-dihydro-2H-furo[2,3-g]indazole-7-carboxamide,
N-(2,4-difluorophenyl)-8-methyl-2-(4-methylbenzyl)-4,5-dihydro-2H-furo[2,3-g]indazole-7-carboxamide,
8-methyl-2-(4-methylbenzyl)-N-[2-(2-methylpiperidin-1-yl)ethyl]-4,5-dihydro-2H-furo[2,3-g]indazole-7-carboxamide,
2-(2-chlorobenzyl)-8-methyl-N-[2-(2-thienyl)ethyl]-4,5-dihydro-2H-furo[2,3-g]indazole-7-carboxamide,
[2-(3-chlorobenzyl)-8-methyl-4,5-dihydro-2H-furo[2,3-g]indazol-7-yl](4-methylpiperazin-1-yl)methanone,
N-(2,5-difluorophenyl)-8-methyl-2-(4-methylbenzyl)-4,5-dihydro-2H-furo[2,3-g]indazole-7-carboxamide,
2-(4-chlorobenzyl)-8-methyl-N-{2-[4-(methylsulfanyl)phenyl]ethyl}-4,5-dihydro-2H-furo[2,3-g]indazole-7-carboxamide,
2-(3-chlorobenzyl)-N-(4-isopropoxybenzyl)-8-methyl-4,5-dihydro-2H-furo[2,3-g]indazole-7-carboxamide,
2-(3-chlorobenzyl)-8-methyl-N-(pyridin-3-ylmethyl)-4,5-dihydro-2H-furo[2,3-g]indazole-7-carboxamide,
ethyl 4-({[8-methyl-2-(4-methylbenzyl)-4,5-dihydro-2H-furo[2,3-g]indazol-7-yl]carbonyl}amino)benzoate,
2-(4-chlorobenzyl)-N-(2-ethyl-6-methylphenyl)-8-methyl-4,5-dihydro-2H-furo[2,3-g]indazole-7-carboxamide,
2-(4-chlorobenzyl)-N-(2,5-difluorophenyl)-8-methyl-4,5-dihydro-2H-furo[2,3-g]indazole-7-carboxamide,
N-(3-chloro-2-methylphenyl)-8-methyl-2-(4-methylbenzyl)-4,5-dihydro-2H-furo[2,3-g]indazole-7-carboxamide,
N-(5-chloro-2-methylphenyl)-8-methyl-2-(4-methylbenzyl)-4,5-dihydro-2H-furo[2,3-g]indazole-7-carboxamide,
2-(2-chlorobenzyl)-8-methyl-N-(3-phenylpropyl)-4,5-dihydro-2H-furo[2,3-g]indazole-7-carboxamide,
2-(3-chlorobenzyl)-N-(3,4-dimethylphenyl)-8-methyl-4,5-dihydro-2H-furo[2,3-g]indazole-7-carboxamide,
3,4-dihydroisoquinolin-2(1H)-yl[8-methyl-2-(4-methylbenzyl)-4,5-dihydro-2H-furo[2,3-g]indazol-7-yl]methanone,
N-[2-(azepan-1-yl)ethyl]-8-methyl-2-(4-methylbenzyl)-4,5-dihydro-2H-furo[2,3-g]indazole-7-carboxamide,
2-(2-chlorobenzyl)-8-methyl-N-(pyridin-3-ylmethyl)-4,5-dihydro-2H-furo[2,3-g]indazole-7-carboxamide,
N-[3-(azepan-1-yl)propyl]-8-methyl-2-(4-methylbenzyl)-4,5-dihydro-2H-furo[2,3-g]indazole-7-carboxamide,
2-(3-chlorobenzyl)-8-methyl-N-(2-methylphenyl)-4,5-dihydro-2H-furo[2,3-g]indazole-7-carboxamide,
2-(3-chlorobenzyl)-N-(4-fluorobenzyl)-8-methyl-4,5-dihydro-2H-furo[2,3-g]indazole-7-carboxamide,
2-(3-chlorobenzyl)-N-(4-ethylbenzyl)-8-methyl-4,5-dihydro-2H-furo[2,3-g]indazole-7-carboxamide,
2-(4-chlorobenzyl)-N-(3,4-dimethylphenyl)-8-methyl-4,5-dihydro-2H-furo[2,3-g]indazole-7-carboxamide,
N-(1,3-benzodioxol-5-ylmethyl)-2-(4-chlorobenzyl)-8-methyl-4,5-dihydro-2H-furo[2,3-g]indazole-7-carboxamide,
2-(2-chlorobenzyl)-N-(4-ethylbenzyl)-8-methyl-4,5-dihydro-2H-furo[2,3-g]indazole-7-carboxamide,
2-(3-chlorobenzyl)-N-(2-fluorophenyl)-8-methyl-4,5-dihydro-2H-furo[2,3-g]indazole-7-carboxamide,
N-(1,3-benzodioxol-5-ylmethyl)-8-methyl-2-(4-methylbenzyl)-4,5-dihydro-2H-furo[2,3-g]indazole-7-carboxamide,
N-(3-acetylphenyl)-8-methyl-2-(4-methylbenzyl)-4,5-dihydro-2H-furo[2,3-g]indazole-7-carboxamide,
2-(4-chlorobenzyl)-N-(2,4-dimethylphenyl)-8-methyl-4,5-dihydro-2H-furo[2,3-g]indazole-7-carboxamide,
2-(2-chlorobenzyl)-N-(4-methoxyphenyl)-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](4-methylpiperazin-1-yl)methanone,
N-(2-chloro-4-methylphenyl)-8-methyl-2-(4-methylbenzyl)-4,5-dihydro-2H-furo[2,3-g]indazole-7-carboxamide,
2-(4-chlorobenzyl)-N-mesityl-8-methyl-4,5-dihydro-2H-furo[2,3-g]indazole-7-carboxamide,
2-(3-chlorobenzyl)-N-(4-methoxyphenyl)-8-methyl-4,5-dihydro-2H-furo[2,3-g]indazole-7-carboxamide,
2-(4-chlorobenzyl)-N-cyclopentyl-8-methyl-4,5-dihydro-2H-furo[2,3-g]indazole-7-carboxamide,
N-(3-acetylphenyl)-2-(4-chlorobenzyl)-8-methyl-4,5-dihydro-2H-furo[2,3-g]indazole-7-carboxamide,
2-(2-chloro-6-fluorobenzyl)-N-(4-methoxyphenyl)-8-methyl-4,5-dihydro-2H-furo[2,3-g]indazole-7-carboxamide,
2-(4-chlorobenzyl)-N-(2,5-dimethoxyphenyl)-8-methyl-4,5-dihydro-2H-furo[2,3-g]indazole-7-carboxamide,
2-(3-chlorobenzyl)-N-(2,4-dimethoxyphenyl)-8-methyl-4,5-dihydro-2H-furo[2,3-g]indazole-7-carboxamide,
ethyl 1-{[2-(3-chlorobenzyl)-8-methyl-4,5-dihydro-2H-furo[2,3-g]indazol-7-yl]carbonyl}piperidine-4-carboxylate,
2-(2-chlorobenzyl)-N-(2,4-dimethoxyphenyl)-8-methyl-4,5-dihydro-2H-furo[2,3-g]indazole-7-carboxamide,
ethyl 4-({[2-(2-chlorobenzyl)-8-methyl-4,5-dihydro-2H-furo[2,3-g]indazol-7-yl]carbonyl}amino)benzoate,
[2-(3-chlorobenzyl)-8-methyl-4,5-dihydro-2H-furo[2,3-g]indazol-7-yl](4-phenylpiperazin-1-yl)methanone,
2-(2-chlorobenzyl)-N-(2,5-dimethoxyphenyl)-8-methyl-4,5-dihydro-2H-furo[2,3-g]indazole-7-carboxamide,
2-(2-chloro-6-fluorobenzyl)-N-(2,3-dihydro-1,4-benzodioxin-6-yl)-8-methyl-4,5-dihydro-2H-furo[2,3-g]indazole-7-carboxamide,
N-[2-chloro-5-(trifluoromethyl)phenyl]-8-methyl-2-(4-methylbenzyl)-4,5-dihydro-2H-furo[2,3-g]indazole-7-carboxamide,
2-(2-chloro-6-fluorobenzyl)-N-(2,4-dimethoxyphenyl)-8-methyl-4,5-dihydro-2H-furo[2,3-g]indazole-7-carboxamide,
2-(2-chloro-6-fluorobenzyl)-N-(2,5-dimethoxyphenyl)-8-methyl-4,5-dihydro-2H-furo[2,3-g]indazole-7-carboxamide,
N-{3-[4-(4-methoxyphenyl)piperazin-1-yl]propyl}-8-methyl-2-(4-methylbenzyl)-4,5-dihydro-2H-furo[2,3-g]indazole-7-carboxamide,
N-{3-[4-(2,5-dimethylphenyl)piperazin-1-yl]propyl}-8-methyl-2-(4-methylbenzyl)-4,5-dihydro-2H-furo[2,3-g]indazole-7-carboxamide,
N-(2-benzoyl-4-chlorophenyl)-8-methyl-2-(4-methylbenzyl)-4,5-dihydro-2H-furo[2,3-g]indazole-7-carboxamide,
N-{3-[4-(4-fluorophenyl)piperazin-1-yl]propyl}-8-methyl-2-(4-methylbenzyl)-4,5-dihydro-2H-furo[2,3-g]indazole-7-carboxamide,
N-{3-[4-(2-fluorophenyl)piperazin-1-yl]propyl}-8-methyl-2-(4-methylbenzyl)-4,5-dihydro-2H-furo[2,3-g]indazole-7-carboxamide,
2-(4-chlorobenzyl)-8-methyl-N-(pyridin-3-ylmethyl)-4,5-dihydro-2H-furo[2,3-g]indazole-7-carboxamide,
2-(2-chlorobenzyl)-N-(3,4-dimethoxyphenyl)-8-methyl-4,5-dihydro-2H-furo[2,3-g]indazole-7-carboxamide,
2-(3-chlorobenzyl)-8-methyl-N-[3-(2-methylpiperidin-1-yl)propyl]-4,5-dihydro-2H-furo[2,3-g]indazole-7-carboxamide,
2-(4-chlorobenzyl)-N-(2,4-dimethoxyphenyl)-8-methyl-4,5-dihydro-2H-furo[2,3-g]indazole-7-carboxamide,
2-(2-chloro-6-fluorobenzyl)-N-(2,4-difluorophenyl)-8-methyl-4,5-dihydro-2H-furo[2,3-g]indazole-7-carboxamide,
2-(2-chloro-6-fluorobenzyl)-N-(3,5-dimethylphenyl)-8-methyl-4,5-dihydro-2H-furo[2,3-g]indazole-7-carboxamide,
2-(4-chlorobenzyl)-N-(3,5-dimethoxyphenyl)-8-methyl-4,5-dihydro-2H-furo[2,3-g]indazole-7-carboxamide,
2-(3-chlorobenzyl)-N-(3,5-dimethoxyphenyl)-8-methyl-4,5-dihydro-2H-furo[2,3-g]indazole-7-carboxamide,
2-(2-chloro-6-fluorobenzyl)-N-(2-ethylphenyl)-8-methyl-4,5-dihydro-2H-furo[2,3-g]indazole-7-carboxamide,
2-(2-chloro-6-fluorobenzyl)-N-(4-isopropylphenyl)-8-methyl-4,5-dihydro-2H-furo[2,3-g]indazole-7-carboxamide,
8-methyl-2-(4-methylbenzyl)-N-[3-(trifluoromethyl)phenyl]-4,5-dihydro-2H-furo[2,3-g]indazole-7-carboxamide,
2-(2-chloro-6-fluorobenzyl)-N-(3-methoxyphenyl)-8-methyl-4,5-dihydro-2H-furo[2,3-g]indazole-7-carboxamide,
N-(1,3-benzodioxol-5-yl)-2-(4-chlorobenzyl)-8-methyl-4,5-dihydro-2H-furo[2,3-g]indazole-7-carboxamide,
N-[3-(azepan-1-yl)propyl]-2-(3-chlorobenzyl)-8-methyl-4,5-dihydro-2H-furo[2,3-g]indazole-7-carboxamide,
N-(1,3-benzodioxol-5-ylmethyl)-2-(3-chlorobenzyl)-8-methyl-4,5-dihydro-2H-furo[2,3-g]indazole-7-carboxamide,
2-(2-chloro-6-fluorobenzyl)-N-(4-ethoxyphenyl)-8-methyl-4,5-dihydro-2H-furo[2,3-g]indazole-7-carboxamide,
2-(2-chloro-6-fluorobenzyl)-N-(3-fluoro-4-methylphenyl)-8-methyl-4,5-dihydro-2H-furo[2,3-g]indazole-7-carboxamide,
2-(2-chlorobenzyl)-8-methyl-N-{2-[4-(methylsulfanyl)phenyl]ethyl}-4,5-dihydro-2H-furo[2,3-g]indazole-7-carboxamide,
N-(5-bromo-2-methoxybenzyl)-8-methyl-2-(4-methylbenzyl)-4,5-dihydro-2H-furo[2,3-g]indazole-7-carboxamide,
2-(2-chlorobenzyl)-N-(3,5-dimethoxyphenyl)-8-methyl-4,5-dihydro-2H-furo[2,3-g]indazole-7-carboxamide,
2-(2-chloro-6-fluorobenzyl)-N-(2-methoxyphenyl)-8-methyl-4,5-dihydro-2H-furo[2,3-g]indazole-7-carboxamide,
2-(2-chloro-6-fluorobenzyl)-N-(2,5-dimethylphenyl)-8-methyl-4,5-dihydro-2H-furo[2,3-g]indazole-7-carboxamide,
N-[3-(4-benzylpiperidin-1-yl)propyl]-8-methyl-2-(4-methylbenzyl)-4,5-dihydro-2H-furo[2,3-g]indazole-7-carboxamide,
2-(3-chlorobenzyl)-8-methyl-N-{2-[4-(methylsulfanyl)phenyl]ethyl}-4,5-dihydro-2H-furo[2,3-g]indazole-7-carboxamide, or
2-(2-chloro-6-fluorobenzyl)-N-(4-fluorophenyl)-8-methyl-4,5-dihydro-2H-furo[2,3-g]indazole-7-carboxamide.

In accordance with a seventh embodiment of the first aspect, the present invention covers compounds of general formula (I), supra, which are selected from the group consisting of:
8-methyl-2-(4-methylbenzyl)-N-[(2S)-tetrahydrofuran-2-ylmethyl]-4,5-dihydro-2H-furo[2,3-g]indazole-7-carboxamide,
2-(2-chlorobenzyl)-8-methyl-N-(4-methylbenzyl)-4,5-dihydro-2H-furo[2,3-g]indazole-7-carboxamide,
2-(2-chlorobenzyl)-8-methyl-N-[2-(4-methylpiperazin-1-yl)ethyl]-4,5-dihydro-2H-furo[2,3-g]indazole-7-carboxamide,
2-(2-chlorobenzyl)-8-methyl-N-[2-(pyrrolidin-1-yl)ethyl]-4,5-dihydro-2H-furo[2,3-g]indazole-7-carboxamide,
2-(2-chlorobenzyl)-8-methyl-N-[2-(piperidin-1-yl)ethyl]-4,5-dihydro-2H-furo[2,3-g]indazole-7-carboxamide,
N-[2-(azetidin-1-yl)ethyl]-2-(2-chlorobenzyl)-8-methyl-4,5-dihydro-2H-furo[2,3-g]indazole-7-carboxamide,
2-(3-chlorobenzyl)-8-methyl-N-[(2RS)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-(4-methylbenzyl)-4,5-dihydro-2H-furo[2,3-g]indazole-7-carboxamide,
2-(3-chlorobenzyl)-8-methyl-N-[2-(4-methylpiperazin-1-yl)ethyl]-4,5-dihydro-2H-furo[2,3-g]indazole-7-carboxamide,
2-(3-chlorobenzyl)-8-methyl-N-[2-(pyrrolidin-1-yl)ethyl]-4,5-dihydro-2H-furo[2,3-g]indazole-7-carboxamide,
2-(3-chlorobenzyl)-8-methyl-N-[2-(piperidin-1-yl)ethyl]-4,5-dihydro-2H-furo[2,3-g]indazole-7-carboxamide,
N-[(2R)-1,4-dioxan-2-ylmethyl]-8-methyl-2-(4-methylbenzyl)-4,5-dihydro-2H-furo[2,3-g]indazole-7-carboxamide,
N-[2-(azetidin-1-yl)ethyl]-2-(3-chlorobenzyl)-8-methyl-4,5-dihydro-2H-furo[2,3-g]indazole-7-carboxamide,
2-(4-chlorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(4-chlorobenzyl)-8-methyl-N-(4-methylbenzyl)-4,5-dihydro-2H-furo[2,3-g]indazole-7-carboxamide,
2-(4-chlorobenzyl)-8-methyl-N-[2-(4-methylpiperazin-1-yl)ethyl]-4,5-dihydro-2H-furo[2,3-g]indazole-7-carboxamide,
2-(4-chlorobenzyl)-8-methyl-N-[2-(pyrrolidin-1-yl)ethyl]-4,5-dihydro-2H-furo[2,3-g]indazole-7-carboxamide,
2-(4-chlorobenzyl)-8-methyl-N-[2-(piperidin-1-yl)ethyl]-4,5-dihydro-2H-furo[2,3-g]indazole-7-carboxamide,
N-[2-(azetidin-1-yl)ethyl]-2-(4-chlorobenzyl)-8-methyl-4,5-dihydro-2H-furo[2,3-g]indazole-7-carboxamide,
2-(4-chlorobenzyl)-8-methyl-N-(1,2-oxazol-3-ylmethyl)-4,5-dihydro-2H-furo[2,3-g]indazole-7-carboxamide,
2-(4-chlorobenzyl)-8-methyl-N-(1,2-oxazol-5-ylmethyl)-4,5-dihydro-2H-furo[2,3-g]indazole-7-carboxamide,
N-[2-(4-acetylpiperazin-1-yl)-2-oxoethyl]-2-(4-chlorobenzyl)-8-methyl-4,5-dihydro-2H-furo[2,3-g]indazole-7-carboxamide,
8-methyl-2-(4-methylbenzyl)-N-(1,2-oxazol-3-ylmethyl)-4,5-dihydro-2H-furo[2,3-g]indazole-7-carboxamide,
2-(4-chlorobenzyl)-N-[2-(ethylamino)-2-oxoethyl]-8-methyl-4,5-dihydro-2H-furo[2,3-g]indazole-7-carboxamide,
2-(4-chlorobenzyl)-N-[2-(diethylamino)-2-oxoethyl]-8-methyl-4,5-dihydro-2H-furo[2,3-g]indazole-7-carboxamide,
2-(4-chlorobenzyl)-N-{2-[(2-methoxyethyl)amino]-2-oxoethyl}-8-methyl-4,5-dihydro-2H-furo[2,3-g]indazole-7-carboxamide,
2-(4-chlorobenzyl)-N-[2-(cyclopropylamino)-2-oxoethyl]-8-methyl-4,5-dihydro-2H-furo[2,3-g]indazole-7-carboxamide,
2-(4-chlorobenzyl)-8-methyl-N-[2-oxo-2-(pyrrolidin-1-yl)ethyl]-4,5-dihydro-2H-furo[2,3-g]indazole-7-carboxamide,
2-(4-chlorobenzyl)-8-methyl-N-[2-(morpholin-4-yl)-2-oxoethyl]-4,5-dihydro-2H-furo[2,3-g]indazole-7-carboxamide,
2-(4-chlorobenzyl)-N-[2-(dimethylamino)-2-oxoethyl]-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](piperidin-1-yl)methanone,
2-(4-chlorobenzyl)-N-(2-hydroxyethyl)-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](2,6-dimethylmorpholin-4-yl)methanone,
8-methyl-2-(2-methylbenzyl)-N-[(2S)-tetrahydrofuran-2-ylmethyl]-4,5-dihydro-2H-furo[2,3-g]indazole-7-carboxamide,
[2-(4-chlorobenzyl)-8-methyl-4,5-dihydro-2H-furo[2,3-g]indazol-7-yl](4-hydroxypiperidin-1-yl)methanone,
2-(4-chlorobenzyl)-8-methyl-N-(2-phenylethyl)-4,5-dihydro-2H-furo[2,3-g]indazole-7-carboxamide,
2-(4-chlorobenzyl)-N-(2-hydroxyethyl)-N,8-dimethyl-4,5-dihydro-2H-furo[2,3-g]indazole-7-carboxamide,
2-(4-chlorobenzyl)-N-(2-methoxyethyl)-8-methyl-4,5-dihydro-2H-furo[2,3-g]indazole-7-carboxamide,
2-(4-chlorobenzyl)-N-(3-hydroxypropyl)-8-methyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}-L-prolinamide,
1-(4-{[2-(4-chlorobenzyl)-8-methyl-4,5-dihydro-2H-furo[2,3-g]indazol-7-yl]carbonyl}piperazin-1-yl)ethanone,
2-(4-chlorobenzyl)-N-(cyclopropylmethyl)-8-methyl-4,5-dihydro-2H-furo[2,3-g]indazole-7-carboxamide,
2-(4-chlorobenzyl)-8-methyl-N-[2-(morpholin-4-yl)ethyl]-4,5-dihydro-2H-furo[2,3-g]indazole-7-carboxamide,
2-(4-chlorobenzyl)-8-methyl-N-(pyridin-2-ylmethyl)-4,5-dihydro-2H-furo[2,3-g]indazole-7-carboxamide,
N-[(2R)-1,4-dioxan-2-ylmethyl]-8-methyl-2-(2-methylbenzyl)-4,5-dihydro-2H-furo[2,3-g]indazole-7-carboxamide,
2-(4-chlorobenzyl)-8-methyl-N-(pyridin-4-ylmethyl)-4,5-dihydro-2H-furo[2,3-g]indazole-7-carboxamide,
N-(2-amino-2-oxoethyl)-2-(4-chlorobenzyl)-8-methyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}piperidine-3-carboxamide,
[2-(4-chlorobenzyl)-8-methyl-4,5-dihydro-2H-furo[2,3-g]indazol-7-yl](3,4-dihydroisoquinolin-2(1H)-yl)methanone,
2-(4-chlorobenzyl)-N-(2-cyanoethyl)-8-methyl-4,5-dihydro-2H-furo[2,3-g]indazole-7-carboxamide,
2-(4-chlorobenzyl)-N-(2-furylmethyl)-N,8-dimethyl-4,5-dihydro-2H-furo[2,3-g]indazole-7-carboxamide,
2-(4-chlorobenzyl)-N,8-dimethyl-N-(pyridin-3-ylmethyl)-4,5-dihydro-2H-furo[2,3-g]indazole-7-carboxamide,
2-(4-chlorobenzyl)-N-(2-cyanopropan-2-yl)-8-methyl-4,5-dihydro-2H-furo[2,3-g]indazole-7-carboxamide,
2-(4-chlorobenzyl)-N,8-dimethyl-N-(pyridin-4-ylmethyl)-4,5-dihydro-2H-furo[2,3-g]indazole-7-carboxamide,
N-(4-acetamidobenzyl)-2-(4-chlorobenzyl)-8-methyl-4,5-dihydro-2H-furo[2,3-g]indazole-7-carboxamide,
tert-butyl (2RS)-2-[({[8-methyl-2-(2-methylbenzyl)-4,5-dihydro-2H-furo[2,3-g]indazol-7-yl]carbonyl}amino)methyl]pyrrolidine-1-carboxylate,
[2-(4-chlorobenzyl)-8-methyl-4,5-dihydro-2H-furo[2,3-g]indazol-7-yl](1,1-dioxidothiomorpholin-4-yl)methanone,
2-(4-chlorobenzyl)-8-methyl-N-(tetrahydro-2H-pyran-4-ylmethyl)-4,5-dihydro-2H-furo[2,3-g]indazole-7-carboxamide,
2-(4-chlorobenzyl)-8-methyl-N-(tetrahydro-2H-pyran-4-yl)-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}piperidine-4-carboxamide,
2-(4-chlorobenzyl)-8-methyl-N-[(1-methyl-1H-pyrrol-2-yl)methyl]-4,5-dihydro-2H-furo[2,3-g]indazole-7-carboxamide,
2-(4-chlorobenzyl)-8-methyl-N-(1,3-thiazol-2-ylmethyl)-4,5-dihydro-2H-furo[2,3-g]indazole-7-carboxamide,
2-(4-chlorobenzyl)-8-methyl-N-[2-(methylsulfinyl)ethyl]-4,5-dihydro-2H-furo[2,3-g]indazole-7-carboxamide,
2-(4-chlorobenzyl)-N,8-dimethyl-N-(pyridin-2-ylmethyl)-4,5-dihydro-2H-furo[2,3-g]indazole-7-carboxamide,
2-(4-chlorobenzyl)-N-(2-methoxyethyl)-N,8-dimethyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}-D-prolinamide,
8-methyl-2-(2-methylbenzyl)-N-[(2RS)-pyrrolidin-2-ylmethyl]-4,5-dihydro-2H-furo[2,3-g]indazole-7-carboxamide,
[2-(4-chlorobenzyl)-8-methyl-4,5-dihydro-2H-furo[2,3-g]indazol-7-yl][4-(hydroxymethyl)piperidin-1-yl]methanone,
2-(4-chlorobenzyl)-N-(2,2-difluoroethyl)-8-methyl-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
2-(4-chlorobenzyl)-8-methyl-N-[(1-methyl-1H-pyrazol-3-yl)methyl]-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
2-(4-chlorobenzyl)-8-methyl-N-[(1-methyl-1H-pyrazol-5-yl)methyl]-4,5-dihydro-2H-furo[2,3-g]indazole-7-carboxamide,
2-(4-chlorobenzyl)-N,8-dimethyl-N-[(1-methyl-1H-pyrazol-3-yl)methyl]-4,5-dihydro-2H-furo[2,3-g]indazole-7-carboxamide,
2-(4-chlorobenzyl)-N,8-dimethyl-N-[(1-methyl-1H-pyrazol-5-yl)methyl]-4,5-dihydro-2H-furo[2,3-g]indazole-7-carboxamide,
2-(4-chlorobenzyl)-8-methyl-N-(1H-pyrazol-3-ylmethyl)-4,5-dihydro-2H-furo[2,3-g]indazole-7-carboxamide,
2-(4-chlorobenzyl)-N-(1H-imidazol-2-ylmethyl)-8-methyl-4,5-dihydro-2H-furo[2,3-g]indazole-7-carboxamide,
2-(4-chlorobenzyl)-8-methyl-N-[(1-methyl-1H-imidazol-5-yl)methyl]-4,5-dihydro-2H-furo[2,3-g]indazole-7-carboxamide,
N-(4-carbamoylbenzyl)-2-(4-chlorobenzyl)-8-methyl-4,5-dihydro-2H-furo[2,3-g]indazole-7-carboxamide,
8-methyl-2-(2-methylbenzyl)-N-(1,2-oxazol-3-ylmethyl)-4,5-dihydro-2H-furo[2,3-g]indazole-7-carboxamide,
[2-(4-chlorobenzyl)-8-methyl-4,5-dihydro-2H-furo[2,3-g]indazol-7-yl][4-(methylsulfonyl)piperazin-1-yl]methanone,
2-(4-chlorobenzyl)-N,8-dimethyl-N-[(1-methyl-1H-pyrazol-4-yl)methyl]-4,5-dihydro-2H-furo[2,3-g]indazole-7-carboxamide,
2-(4-chlorobenzyl)-8-methyl-N-[(1-methyl-1H-pyrazol-4-yl)methyl]-4,5-dihydro-2H-furo[2,3-g]indazole-7-carboxamide,
2-(4-chlorobenzyl)-N-(2-hydroxy-2-methylpropyl)-8-methyl-4,5-dihydro-2H-furo[2,3-g]indazole-7-carboxamide,
2-(4-chlorobenzyl)-N-[(1,1-dioxidotetrahydrothiophen-3-yl)methyl]-8-methyl-4,5-dihydro-2H-furo[2,3-g]indazole-7-carboxamide,
2-(4-chlorobenzyl)-8-methyl-N-[(1-methyl-1H-imidazol-2-yl)methyl]-4,5-dihydro-2H-furo[2,3-g]indazole-7-carboxamide,
2-(4-chlorobenzyl)-N-(1H-imidazol-2-ylmethyl)-N,8-dimethyl-4,5-dihydro-2H-furo[2,3-g]indazole-7-carboxamide,
(3-benzylazetidin-1-yl)[2-(4-chlorobenzyl)-8-methyl-4,5-dihydro-2H-furo[2,3-g]indazol-7-yl]methanone,
N-(3-amino-3-oxopropyl)-2-(4-chlorobenzyl)-8-methyl-4,5-dihydro-2H-furo[2,3-g]indazole-7-carboxamide,
2-(4-chlorobenzyl)-8-methyl-N-(pyrazolo[1,5-a]pyridin-3-ylmethyl)-4,5-dihydro-2H-furo[2,3-g]indazole-7-carboxamide,
2-(2-chlorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-(6-oxopiperidin-3-yl)-4,5-dihydro-2H-furo[2,3-g]indazole-7-carboxamide,
3-azabicyclo[3.1.0]hexan-3-yl[2-(4-chlorobenzyl)-8-methyl-4,5-dihydro-2H-furo[2,3-g]indazol-7-yl]methanone,
2-(2-chlorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2RS)-tetrahydrofuran-2-ylmethyl]-4,5-dihydro-2H-furo[2,3-g]indazole-7-carboxamide,
2-(2-chlorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[2-(4-methylpiperazin-1-yl)ethyl]-4,5-dihydro-2H-furo[2,3-g]indazole-7-carboxamide,
2-(2-chloro-4-fluorobenzyl)-8-methyl-N-(1,2-oxazol-3-ylmethyl)-4,5-dihydro-2H-furo[2,3-g]indazole-7-carboxamide, or
2-[(1RS)-1-(2-fluorophenyl)ethyl]-8-methyl-N-[(2S)-tetrahydrofuran-2-ylmethyl]-4,5-dihydro-2H-furo[2,3-g]indazole-7-carboxamide.

In accordance with an eighth embodiment of the first aspect, the present invention covers compounds of general formula (I), supra, which are selected from the group consisting of:
N-[(2R)-1,4-dioxan-2-ylmethyl]-8-methyl-2-(4-methylbenzyl)-4,5-dihydro-2H-furo[2,3-g]indazole-7-carboxamide,
N-[(2R)-1,4-dioxan-2-ylmethyl]-8-methyl-2-(2-methylbenzyl)-4,5-dihydro-2H-furo[2,3-g]indazole-7-carboxamide,
2-(2-chlorobenzyl)-8-methyl-N-[(2R*)-tetrahydrofuran-2-ylmethyl]-4,5-dihydro-2H-furo[2,3-g]indazole-7-carboxamide,
8-methyl-2-(4-methylbenzyl)-N-[(2S)-tetrahydrofuran-2-ylmethyl]-4,5-dihydro-2H-furo[2,3-g]indazole-7-carboxamide,
8-methyl-2-(2-methylbenzyl)-N-[(2S)-tetrahydrofuran-2-ylmethyl]-4,5-dihydro-2H-furo[2,3-g]indazole-7-carboxamide,
2-(2-chlorobenzyl)-8-methyl-N-(4-methylbenzyl)-4,5-dihydro-2H-furo[2,3-g]indazole-7-carboxamide,
2-(2-chlorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-[(2RS)-tetrahydrofuran-2-ylmethyl]-4,5-dihydro-2H-furo[2,3-g]indazole-7-carboxamide,
2-(2-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-[(2RS)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-(4-methylbenzyl)-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
8-methyl-2-(4-methylbenzyl)-N-(1,2-oxazol-3-ylmethyl)-4,5-dihydro-2H-furo[2,3-g]indazole-7-carboxamide,
2-(4-chlorobenzyl)-8-methyl-N-(1,2-oxazol-3-ylmethyl)-4,5-dihydro-2H-furo[2,3-g]indazole-7-carboxamide,
2-(2-chloro-4-fluorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-[(1-methyl-1H-pyrazol-3-yl)methyl]-4,5-dihydro-2H-furo[2,3-g]indazole-7-carboxamide,
2-(4-chlorobenzyl)-8-methyl-N-(1,3-thiazol-2-ylmethyl)-4,5-dihydro-2H-furo[2,3-g]indazole-7-carboxamide,
2-(2-chlorobenzyl)-8-methyl-N-[2-(4-methylpiperazin-1-yl)ethyl]-4,5-dihydro-2H-furo[2,3-g]indazole-7-carboxamide,
8-methyl-2-(2-methylbenzyl)-N-(1,2-oxazol-3-ylmethyl)-4,5-dihydro-2H-furo[2,3-g]indazole-7-carboxamide,
2-(2-chloro-4-fluorobenzyl)-8-methyl-N-(1,2-oxazol-3-ylmethyl)-4,5-dihydro-2H-furo[2,3-g]indazole-7-carboxamide,
2-(4-chlorobenzyl)-8-methyl-N-(pyridin-2-ylmethyl)-4,5-dihydro-2H-furo[2,3-g]indazole-7-carboxamide,
2-(2-chlorobenzyl)-8-methyl-N-[2-(pyrrolidin-1-yl)ethyl]-4,5-dihydro-2H-furo[2,3-g]indazole-7-carboxamide,
2-(4-chlorobenzyl)-8-methyl-N-[2-(4-methylpiperazin-1-yl)ethyl]-4,5-dihydro-2H-furo[2,3-g]indazole-7-carboxamide,
2-(3-chlorobenzyl)-8-methyl-N-[2-(4-methylpiperazin-1-yl)ethyl]-4,5-dihydro-2H-furo[2,3-g]indazole-7-carboxamide,
2-(4-chlorobenzyl)-8-methyl-N-(4-methylbenzyl)-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
2-(2-chlorobenzyl)-8-methyl-N-[2-(piperidin-1-yl)ethyl]-4,5-dihydro-2H-furo[2,3-g]indazole-7-carboxamide,
2-(4-chlorobenzyl)-N-(2-methoxyethyl)-8-methyl-4,5-dihydro-2H-furo[2,3-g]indazole-7-carboxamide,
2-(4-chlorobenzyl)-N-[2-(ethylamino)-2-oxoethyl]-8-methyl-4,5-dihydro-2H-furo[2,3-g]indazole-7-carboxamide,
2-(4-chlorobenzyl)-8-methyl-N-(1H-pyrazol-3-ylmethyl)-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
8-methyl-2-(2-methylbenzyl)-N-[(2RS)-pyrrolidin-2-ylmethyl]-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
N-(4-carbamoylbenzyl)-2-(4-chlorobenzyl)-8-methyl-4,5-dihydro-2H-furo[2,3-g]indazole-7-carboxamide,
2-[(1RS)-1-(2-fluorophenyl)ethyl]-8-methyl-N-[(2S)-tetrahydrofuran-2-ylmethyl]-4,5-dihydro-2H-furo[2,3-g]indazole-7-carboxamide,
2-(3-chlorobenzyl)-8-methyl-N-[2-(piperidin-1-yl)ethyl]-4,5-dihydro-2H-furo[2,3-g]indazole-7-carboxamide,
N-(4-acetamidobenzyl)-2-(4-chlorobenzyl)-8-methyl-4,5-dihydro-2H-furo[2,3-g]indazole-7-carboxamide,
2-(2-chloro-4-fluorobenzyl)-8-methyl-N-[2-(4-methylpiperazin-1-yl)ethyl]-4,5-dihydro-2H-furo[2,3-g]indazole-7-carboxamide, or 2-(4-chlorobenzyl)-8-methyl-N-[(1-methyl-1H-pyrrol-2-yl)methyl]-4,5-dihydro-2H-furo[2,3-g]indazole-7-carboxamide.

The present invention covers any sub-combination within any embodiment or aspect of the present invention of compounds of general formula (I), supra.

The present invention covers any sub-combination within any embodiment or aspect of the present invention of intermediate compounds of general formula (II).

The present invention covers the compounds of general formula (I) which are disclosed in the Example Section of this text, infra.

The compounds according to the invention of general formula (I) can be prepared according to the following schemes 1 and 2. The schemes and procedures described below illustrate synthetic routes to the compounds of general formula (I) of the invention and are not intended to be limiting. It is clear to the person skilled in the art that the order of transformations as exemplified in schemes 1 and 2 can be modified in various ways. The order of transformations exemplified in these schemes is therefore not intended to be limiting. In addition, interconversion of any of the substituents R¹, R², R³, R⁴, R⁵, R⁶, R^{7a} or R^{7b} can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art. Specific examples are described in the subsequent paragraphs.

Routes for the preparation of compounds of general formula (I) and corresponding intermediates are described in schemes 1 and 2.

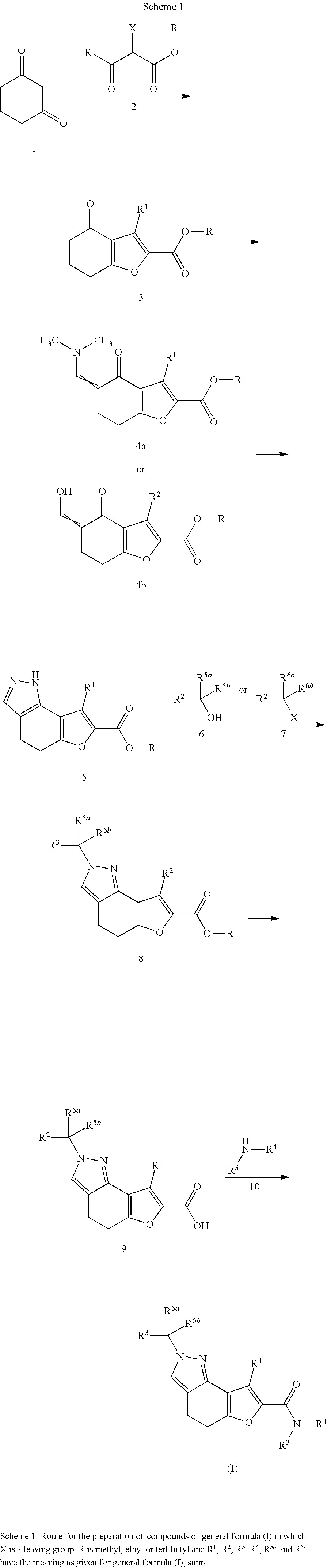

Tetrahydrobenzofuranes of general formula (3) can be obtained via aldol condensation of (1) and (2) followed by intramolecular cyclisation according to the procedures described by Stetter at al. (Chem. Ber. 1960, 93, 603-607) as depicted in Scheme 1. Compounds (1) and (2) are either commercially available or can be prepared according to procedures available from the public domain, as understandable to the person skilled in the art. Depending on the reactivity of the involved centers the regioisomer of (3) can be obtained [i.e. in cases where nucleophilic displacement of the leaving group of (2) by the acidic methylene unit of (1) is taking place prior to intramolecular condensation with the ketone moiety of (2)].

In general, 1,3-diketones of formula (1) can be reacted with alpha-carbonylesters of general formula (2) in the presence of inorganic bases like sodium hydroxide or potassium hydroxide, preferably potassium hydroxide, in protic solvents such as for example methanol, ethanol or water or mixtures thereof, preferably a mixture of the alcohol incorporated in ester (2) and water, at temperatures between 0° C. and the boiling point of the solvent (mixture), preferably between room temperature and 50° C. The reaction times vary between 15 hours and several days. It is usually necessary to isomerize the primary formed cyclisation products to the tetrahydrobenzofuranes of general formula (3) by treatment with acids such as aqueous hydrochloric acid at pH 1-4 at temperatures between 0° C. and the boiling point of the solvent (mixture), preferably at room temperature, for 1-6 hours.

Alternatively, (1) and (2) may be reacted in the presence of organic bases like triethylamine in aprotic solvents like dichloromethane, dichloroethane or tetrahydrofuran, preferably dichloromethane or dichloroethane, at temperatures between room temperature and the boiling point of the solvent, preferably at 40-60° C. (pressure tube), for 12-72 h followed by treatment with acids such as aqueous hydrochloric acid at pH 1-4 at temperatures between 0° C. and the boiling point of the solvent (mixture), preferably at room temperature, for 3-24 hours.

Alternatively, (1) and (2) may be reacted without further additives in toluene at temperatures between room temperature and 120° C., preferably at 80-120° C. for 12-20 hours.

Enamines of general formula (4a) can be synthesized from tetrahydrobenzofuranes of general formula (3) by alpha-methylation with electrophiles like 1-tert-butoxy-N,N,N′,N′ tetramethylmethanediamine (Bredereck's reagent) or 1,1-dimethoxy-N,N-dimethylmethanamine, preferably 1-tert-butoxy-N,N,N′,N′-tetramethylmethanediamine, in aprotic solvents like benzene, toluene or dioxane, preferably toluene, at temperatures between room temperature and the boiling point of the solvent, preferably at 100-110° C., for 15 hours or up to several days.

Alternatively, tetrahydrobenzofuranes of general formula (3) can be transferred to alpha-hydroxymethyleneketones of general formula (4b) by formylation with formic acid derivatives such as ethyl formate or methyl formate in the presence of bases such as sodium methylate, sodium ethylate, potassium tert-butoxide or sodium hydride in solvents such as methanol, ethanol, toluene or tetrahydrofuran or mixtures thereof at temperatures between 0° C. and the boiling point of the solvent (mixture), preferably between room temperature and 50° C., for 1-18 hours.

Furoindazoles of general formula (5) can be obtained starting from either enamines of general formula (4a) or alpha-hydroxymethyleneketones of general formula (4b) by reacting (4a) or (4b) with hydrazine or hydrazine derivatives such as hydrazine hydrates or hydrazine salts, preferably hydrazine hydrate or hydrazine dihydrochloride, in polar protic solvents like ethanol or water or mixtures thereof, preferably ethanol/water mixtures, at temperatures between room temperature and the boiling point of the solvent (mixture), preferably at 70-80° C., for 4-18 hours.

2-Substituted furoindazole esters of general formula (8) can be synthesized from furoindazoles of general formula (5) either by Mitsunobu reaction with alcohols of general formula (6) in the presence of activating reagents such as diisopropyl azodicarboxylate (DIAD) or N,N,N′,N′-tetramethylazodicarboxamide (TMAD) and a tertiary posphine such as triphenylphosphine or tri-n-butylphosphine, preferably a combination of TMAD and tri-n-butylphosphine, in aprotic solvents such as tetrahydrofuran or toluene, preferably toluene, at temperatures between room temperature and the boiling point of the solvent, preferably at room temperature, for 12-48 hours. Alternatively, 2-substituted furoindazoles of general formula (8) can be synthesized from furoindazoles of general formula (5) by reaction with electrophiles of general formula (7) such as alkyl halides or alkyl tosylates or alkyl mesylates, preferably alkyl bromides, in the presence of an inorganic base such as potassium carbonate or in the presence of an organic base such as triethylamine or N,N-diisopropylethylamine, preferably potassium carbonate, in a polar, aprotic solvent such as acetonitrile or ethyl acetate, preferably acetonitrile, at temperatures between room temperature and the boiling point of the solvent, preferably at 60-75° C. It can be beneficial to add a catalyst like 4-dimethylaminopyridine (DMAP) to the mixture. Generally, depending on the reactivity of the involved centers the 1-substituted regioisomer of (8) can be obtained in certain cases as well.

Carboxylic acids of general formula (9) may be obtained from carboxylic esters of formula (8), wherein R has the meaning of methyl or ethyl, by saponification with inorganic bases such as lithium hydroxide, potassium hydroxide or sodium hydroxide, preferably lithium hydroxide, in a suitable solvent such as methanol, ethanol, tetrahydrofuran, water or mixtures thereof, preferably a mixture of the alcohol incorporated in ester (8), THF and water, at temperatures between 0° C. and the boiling point of the solvent (mixture), typically at 70° C., for 4-48 hours. In case R has the meaning of tert-butyl in carboxylic esters of formula (8), the ester may be hydrolysed using an organic or inorganic acid like trifluoroacetic acid or hydrogen chloride as solution in inert solvents like dichloromethane or 1,4-dioxane at temperatures between 0° C. and the boiling point of the solvent (mixture), typically at 25° C., for 4-48 hours.

Furoindazoles of general formula (I) may be synthesized from suitably functionalized carboxylic acids of general formula (9) by reaction with appropriate amines HN(R⁵)(R⁶) (10). For amide formation, however, all processes that are known from peptide chemistry to the person skilled in the art may be applied. The acids of general formula (9) can be reacted with an appropriate amine in aprotic polar solvents, such as for example DMF, acetonitrile or N-methylpyrrolid-2-one via an activated acid derivative, which is obtainable for example with hydroxybenzotriazole and a carbodiimide such as for example diisopropylcarbodiimide, or else with preformed reagents, such as for example O-(7-azabenzotriazol-1-yl)-1,1,3,3-tetramethyluronium hexafluorophosphate (see for example Chem. Comm. 1994, 201-203), or else with activating agents such as dicyclohexylcarbodiimide N,N-dimethylaminopyridine or N-ethyl-N′,N′ dimethylaminopropylcarbodiimide/N,N-dimethylaminopyridine. The addition of a suitable base such as for example N-methylmorpholine, triethylamine or DIPEA may be necessary. In certain cases, the activated acid derivative might be isolated prior to reaction with the appropriate amine. Amide formation may also be accomplished via the acid halide (which can be formed from a carboxylic acid by reaction with e.g. oxalyl chloride, thionyl chloride or sulfuryl chloride), mixed acid anhydride (which can be formed from a carboxylic acid by reaction with e.g. isobutylchloroformate), imidazolide (which can be formed from a carboxylic acid by reaction with e.g. carbonyldiimidazole) or azide (which can be formed from a carboxylic acid by reaction with e.g. diphenylphosphorylazide).

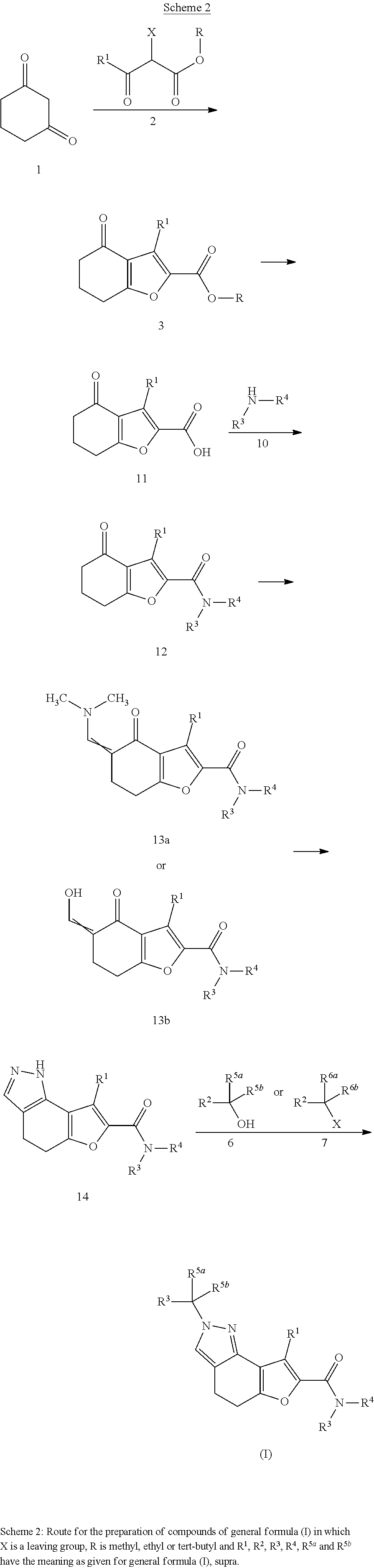

Tetrahydrobenzofuranes of general formula (3) can be obtained via aldol condensation of (1) and (2) via methods already described for Scheme 1.

Carboxylic acids of general formula (11) may be obtained from carboxylic esters of formula (3), wherein R has the meaning of methyl or ethyl, by saponification with inorganic bases such as lithium hydroxide, potassium hydroxide or sodium hydroxide, preferably lithium hydroxide, in a suitable solvent such as methanol, ethanol, tetrahydrofuran, water or mixtures thereof, preferably a mixture of the alcohol incorporated in ester (3), THF and water, at temperatures between 0° C. and the boiling point of the solvent (mixture), typically at 70° C., for 4-48 hours. In case R has the meaning of tert-butyl in carboxylic esters of formula (3), the ester may be hydrolysed using an organic or inorganic acid like trifluoroacetic acid or hydrogen chloride as solution in inert solvents like dichloromethane or 1,4-dioxane at temperatures between 0° C. and the boiling point of the solvent (mixture), typically at 25° C., for 4-48 hours.

Furoamides of general formula (12) may be synthesized from suitably functionalized carboxylic acids of general formula (11) by reaction with appropriate amines HN(R⁵)(R⁶) (10). For amide formation, however, all processes that are known from peptide chemistry to the person skilled in the art may be applied. The acids of general formula (11) can be reacted with an appropriate amine in aprotic polar solvents, such as for example DMF, acetonitrile or N-methylpyrrolid-2-one via an activated acid derivative, which is obtainable for example with hydroxybenzotriazole and a carbodiimide such as for example diisopropylcarbodiimide, or else with preformed reagents, such as for example O-(7-azabenzotriazol-1-yl)-1,1,3,3-tetramethyluronium hexafluorophosphate (see for example Chem. Comm. 1994, 201-203), or else with activating agents such as dicyclohexylcarbodiimide/N,N-dimethylaminopyridine or N-ethyl-N′,N′ dimethylaminopropylcarbodiimide/N,N-dimethylaminopyridine. The addition of a suitable base such as for example N-methylmorpholine, triethylamine or DIPEA may be necessary. In certain cases, the activated acid derivative might be isolated prior to reaction with the appropriate amine. Amide formation may also be accomplished via the acid halide (which can be formed from a carboxylic acid by reaction with e.g. oxalyl chloride, thionyl chloride or sulfuryl chloride), mixed acid anhydride (which can be formed from a carboxylic acid by reaction with e.g. isobutylchloroformate), imidazolide (which can be formed from a carboxylic acid by reaction with e.g. carbonyldiimidazole) or azide (which can be formed from a carboxylic acid by reaction with e.g. diphenylphosphorylazide).

Enamines of general formula (13a) can be synthesized from furoamides of general formula (12) by alpha-methylation with electrophiles like 1-tert-butoxy-N,N,N′,N′-tetramethylmethanediamine (Bredereck's reagent) or 1,1-dimethoxy-N,N-dimethylmethanamine, preferably 1-tert-butoxy-N,N,N′,N′-tetramethylmethanediamine, in aprotic solvents like benzene, toluene or dioxane, preferably toluene, at temperatures between room temperature and the boiling point of the solvent, preferably at 100-110° C., for 15 hours or up to several days.

Alternatively, furoamides of general formula (12) can be transferred to alpha-hydroxymethyleneketones of general formula (13b) by formylation with formic acid derivatives such as ethyl formate or methyl formate in the presence of bases such as sodium methylate, sodium ethylate, potassium tert-butoxide or sodium hydride in solvents such as methanol, ethanol, toluene or tetrahydrofuran or mixtures thereof at temperatures between 0° C. and the boiling point of the solvent (mixture), preferably between room temperature and 50° C., for 1-18 hours.

Furoindazoles of general formula (14) can be obtained starting from either enamines of general formula (13a) or alpha-hydroxymethyleneketones of general formula (13b) by reacting (13a) or (13b) with hydrazine or hydrazine derivatives such as hydrazine hydrates or hydrazine salts, preferably hydrazine hydrate or hydrazine dihydrochloride, in polar protic solvents like ethanol or water or mixtures thereof, preferably ethanol/water mixtures, at temperatures between room temperature and the boiling point of the solvent (mixture), preferably at 70-80° C., for 4-18 hours.

Furoindazoles of general formula (I)) can be synthesized from furoindazoles of general formula (14) either by Mitsunobu reaction with alcohols of general formula (6) in the presence of activating reagents such as diisopropyl azodicarboxylate (DIAD) or N,N,N′,N′-tetramethylazodicarboxamide (TMAD) and a tertiary posphine such as triphenylphosphine or tri-n-butylphosphine, preferably a combination of TMAD and tri-n-butylphosphine, in aprotic solvents such as tetrahydrofuran or toluene, preferably toluene, at temperatures between room temperature and the boiling point of the solvent, preferably at room temperature, for 12-48 hours. Alternatively, furoindazoles of general formula (I) can be synthesized from furoindazoles of general formula (14) by reaction with electrophiles of general formula (7) such as alkyl halides or alkyl tosylates or alkyl mesylates, preferably alkyl bromides, in the presence of an inorganic base such as potassium carbonate or in the presence of an organic base such as triethylamine or N,N-diisopropylethylamine, preferably potassium carbonate, in a polar, aprotic solvent such as acetonitrile or ethyl acetate, preferably acetonitrile, at temperatures between room temperature and the boiling point of the solvent, preferably at 60-75° C. It can be beneficial to add a catalyst like 4-dimethylaminopyridine (DMAP) to the mixture. Generally, depending on the reactivity of the involved centers the 1-substituted regioisomer of (I) can be obtained in certain cases as well.

Specific examples are described in the Experimental Section.

In accordance with a second aspect, the present invention covers methods of preparing compounds of general formula (I) as defined supra, said methods comprising the step of allowing an intermediate compound of general formula (II):

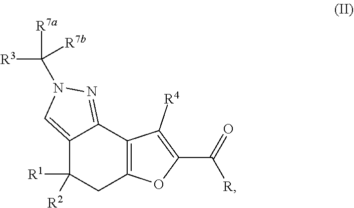

in which R is H or OH or OMe or OEt and R¹, R², R³, R⁴, R^{7a} and R^{7b} are as defined for the compound of general formula (I) as defined supra, to react with a compound of general formula (III):

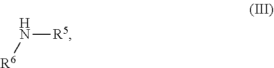

in which R⁵ and R⁶ are as defined for the compound of general formula (I) as defined supra,
thereby giving a compound of general formula (I):

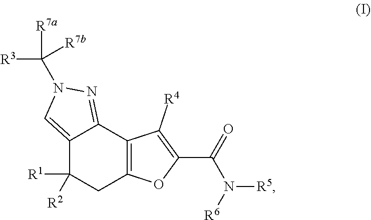

in which R¹, R², R³, R⁵, R⁶, R^{7a} and R^{7b} are as defined supra.

In accordance with a third aspect, the present invention covers methods of preparing compounds of general formula (I) as defined supra, said methods comprising the step of allowing an intermediate compound of general formula (II):

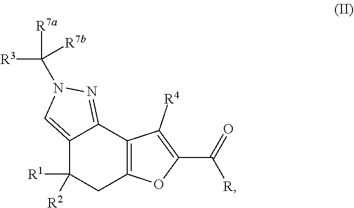

in which R is H, OH, OMe, or OEt and R¹, R², R³, R⁴, R^{7a} and R^{7b} are as defined for the compound of general formula (I) as defined supra,
to react with a compound of general formula (III):

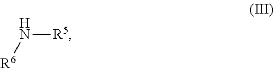

in which R⁵ and R⁶ are as defined for the compound of general formula (I) as defined supra,
thereby giving a compound of general formula (I):

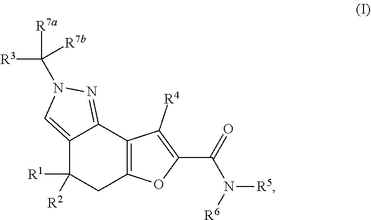

in which R¹, R², R³, R⁵, R⁶, R^{7a} and R^{7b} are as defined supra,
then optionally converting said compound into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

In accordance with a fourth aspect, the present invention covers intermediate compounds which are useful for the preparation of the compounds of general formula (I), supra.

Particularly, the invention covers the intermediate compounds of general formula (II):

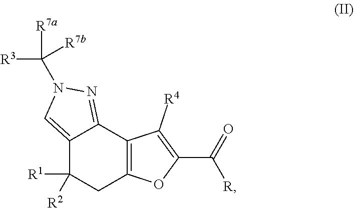

in which R is H or OH or OMe or OEt and R¹, R², R³, R⁴, R^{7a} and R^{7b} are as defined for the compound of general formula (I) supra.

In accordance with a fifth aspect, the present invention covers the use of said intermediate compounds for the preparation of a compound of general formula (I) as defined supra.

Particularly, the invention covers the use of intermediate compounds of general formula (II):

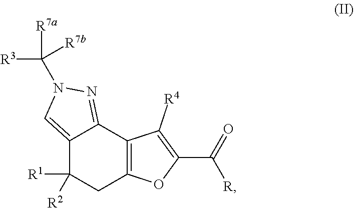

in which R is H or OH or OMe or OEt and R¹, R², R³, R⁴, R^{7a} and R^{7b} are as defined for the compound of general formula (I) supra, for the preparation of a compound of general formula (I) as defined supra.

The present invention covers the intermediate compounds which are disclosed in the Example Section of this text, infra.

The present invention covers any sub-combination within any embodiment or aspect of the present invention of intermediate compounds of general formula (II), supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. Compounds of the present invention have surprisingly been found to be effective antagonists of GPR84 and it is possible therefore that said compounds be used for the treatment or prophylaxis of diseases, in particular of autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

Compounds of the present invention can be utilized to inhibit, antagonize, block, reduce, decrease GPR84 signal transduction, activity and cellular function. This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

In particular of autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

The present invention also provides methods of treating PCOS and symptoms.

These disorders have been well characterized in humans, but also exist with a similar aetiology in other mammals and can be treated by administering pharmaceutical compositions of the present invention.

The term “treating”, or “treatment” as used in the present text is used conventionally, e.g., the management or care of a subject for the purpose of combating, alleviating, reducing, relieving, improving the condition of a disease or disorder, such as PCOS or IPF.

The compounds of the present invention can be used in particular in therapy and prevention, i.e. prophylaxis and treatment of autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

The pharmaceutical activity of the compounds according to the invention can be explained by their activity as GPR84 antagonists.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of diseases, in particular autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals.

In accordance with a further aspect, the present invention covers a method of treatment or prophylaxis of diseases, in particular autoimmune diseases such as multiple sclerosis, psoriasis, psoriatic arthritis, rheumatoid arthritis, ankylosing spondylitis, systemic lupus erythematosus, primary and secondary autoimmune uveitis, inflammatory disorders like endometriosis, inflammatory eye diseases, inflammatory kidney diseases, inflammatory liver diseases like non-alcoholic, alcoholic- and toxic fatty liver diseases, lung diseases like asthma, idiopathic pulmonary fibrosis, chronic obstructive pulmonary disease and metabolic and metabolic-endocrine disorders like metabolic syndrome, insulin resistance, diabetes mellitus type I and type II, and polycystic ovary syndrome (PCOS) disorders, neuropathic and inflammatory pain disorders in humans and animals, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above-mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophilizates, capsules (for example hard or soft gelatine capsules), sugar-coated tablets, granules, pellets, powders, emulsions, suspensions, aerosols or solutions. It is possible to incorporate the compounds according to the invention in crystalline and/or amorphized and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophilizates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

### EXPERIMENTAL SECTION

NMR peak forms are stated as they appear in the spectra, possible higher order effects have not been considered.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Chemical names were generated using the ACD/Name software from ACD/Labs. In some cases, generally accepted names of commercially available reagents were used in place of ACD/Name generated names.

The following Table 1 lists the abbreviations used in this paragraph and in the Examples section as far as they are not explained within the text body. Other abbreviations have their meanings customary per se to the skilled person. The following table lists the abbreviations used herein.

TABLE 1
Abbreviations

| Abbreviation | Meaning |
|---|---|
| br. | broad signal in NMR |
| br. s. | broad singlet |
| CDI | di-1H-imidazol-1-ylmethanone |
| conc. | concentrated |
| CPME | cyclopentyl methyl ether |
| d | doublet |
| dd | doublet of doublets |
| ddd | doublet of doublet of doublets |
| dt | doublet of triplets |
| DCM | dichloromethane |
| DEA | diethylamine |
| DIPEA | N,N-diisopropylethyl amine |
| DMAP | N,N-dimethylpyridin-4-amine |
| DMF | N,N-dimethylformamide |
| DMSO | dimethyl sulfoxide |
| EDC | N-(3-dimethylaminopropyl)-N′-ethylcarbodiimide |
| | hydrochloride |
| ESI | electrospray ionization |
| ESIpos | Positive electrospray ionization |
| ESIneg | Negative electrospray ionization |
| EtOAc | ethyl acetate |
| EtOH | ethanol |
| eq. | equivalent |
| GP | General procedure |
| h | hour(s) |
| HATU | O-(7-azabenzotriazol-1-yl)-1,1,3,3-tetramethyluronium |
| | hexafluorophosphate |
| HCl | hydrochloric acid |
| HCOOH | formic acid |
| HPLC, LC | high performance liquid chromatography |
| LC-MS / LCMS | Liquid chromatography mass spectrometry |
| m | multiplet |
| min | minute(s) |
| MS | mass spectroscopy |
| MeCN | acetonitrile |
| MeOH | methanol |
| NMR | nuclear magnetic resonance |
| q | quartet |
| quint | quintet |
| Rₜ | retention time |
| rt | room temperature |
| s | singlet |
| sept | septet |
| t | triplet |
| TFA | trifluoroacetic acid |
| THF | tetrahydrofuran |
| TMAD | N,N,N′,N′-tetramethylazodicarboxamide |
| UPLC | ultra performance liquid chromatography |
| UPLC-MS | ultra performance liquid chromatography mass |
| | spectrometry |

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

Some compounds of general formula (I), for which the synthesis is not described in the experimental part, are commercially available.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartridges KP-Sil® or KP-NH® in combination with a Biotage auto purifier system (SP4® or Isolera Four®) and eluents such as gradients of hexane/ethyl acetate or DCM/methanol. In some cases, the compounds may be purified by preparative HPLC using for example a Waters auto purifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### UPLC-MS Standard Procedures

Analytical UPLC-MS was performed as described below. The masses (m/z) are reported from the positive mode electrospray ionisation unless the negative mode is indicated (ESI-). In most of the cases method 1 is used. If not, it is indicated.

### Method 1:

Instrument: Waters Acquity UPLC-MS SQD 3001; Column: Acquity UPLC BEH C18 1.7 μm, 50×2.1 mm; Eluent A: water+0.2 vol % ammonia, Eluent B: acetonitrile; Gradient: 0-1.6 min 1-99% B, 1.6-2.0 min 99% B; Flow rate: 0.8 mL/min; Temperature: 60° C.; Injection: 2 μL; DAD scan: 210-400 nm; ELSD.

### Method 2:

Instrument: Waters Acquity UPLC-MS SQD 3001; Column: Acquity UPLC BEH C18 1.7 μm, 50×2.1 mm; Eluent A: water+0.1 vol % formic acid, Eluent B: acetonitrile; Gradient: 0-1.6 min 1-99% B, 1.6-2.0 min 99% B; Flow rate: 0.8 mL/min; Temperature: 60° C.; Injection: 2 μL; DAD scan: 210-400 nm.

### LC-MS Standard Procedures

### Method A:

Instrument: Waters Acquity UPLCMS SingleQuad; Colum: Acquity UPLC BEH C18 1.7 μm, 50×2.1 mm; Eluent A: water+0.2 vol % aqueous ammonia (32%), Eluent B: acetonitrile; Gradient: 0-1.6 min 1-99% B, 1.6-2.0 min 99% B; Flow: 0.8 mL/min; Temperature: 60° C.; DAD scan: 210-400 nm.

Method B: Instrument: pump: Labomatic HD-5000 or HD-3000, head HDK 280, low pressure gradient module ND-B1000; manual injection valve: Rheodyne 3725i038; detector: Knauer Azura UVD 2.15; collector: Labomatic Labocol Vario-4000; column: Chromatorex RP C-18 10 μm, 125×30 mm; eluent A: water+0.2 vol-% ammonia (32%), eluent B: acetonitrile;
gradient A: 0-15 min 1-25% B; flow: 60 ml/min;
gradient B: 0-15 min 10-50% B; flow: 60 ml/min;
gradient C: 0-15 min 15-55% B; flow: 60 ml/min;
gradient D: 0-15 min 30-70% B; flow: 60 ml/min;
gradient E: 0-15 min 40-80% B; flow: 60 ml/min;
gradient F: 0-15 min 65-100% B; flow: 60 ml/min;
temperature: 25° C.; solution: max. 250 mg/2 ml dimethyl sulfoxide; injection: 1×2 ml; Detection: UV 254 nm; Software: SCPA PrepCon5.

Analytical characterization of enantiomers was performed by analytical chiral HPLC. In the description of the individual examples is referred to the applied HPLC procedure.

### Experimental Section—Intermediates

### Intermediate 1:

### Step 1

### ethyl 8-methyl-2-[(4-methylphenyl)methyl]-4,5-dihydro-2H-furo[2,3-g]indazole-7-carboxylate

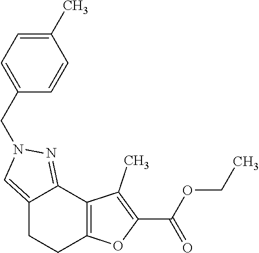 [SMILES: CCOC(=O)c1oc2c(c1C)-c1nn(Cc3ccc(C)cc3)cc1CC2]

A solution of ethyl 8-methyl-4,5-dihydro-1H-furo[2,3-g]indazole-7-carboxylate (commercially available; 1.00 eq., 500 mg, 2.03 mmol; CAS-RN:[903163-04-2]) in acetonitrile (10 mL) was treated with 1-(bromomethyl)-4-methylbenzene (1.50 eq., 564 mg, 3.05 mmol; CAS-RN:[104-81-4]) and potassium carbonate (15.0 eq., 4.21 g, 30.5 mmol; CAS-RN:[584-08-7]) and stirred at 60° C. for three days. The reaction mixture was cooled to rt and filtrated. The filtrate was concentrated under reduced pressure and the residue subjected to column chromatography (SiO₂, hexane/ethyl acetate) to give the title compound (418 mg, 53%).

¹H NMR (400 MHz, DMSO-d6) δ [ppm]: 1.29 (t, 3H), 2.27 (s, 3H), 2.48 (s, 3H), 2.82-2.92 (m, 4H), 4.26 (q, 2H), 5.23 (s, 2H), 7.11-7.20 (m, 4H), 7.57 (s, 1H).

UPLC-MS (Method 1): Rₜ=1.44 min; MS (ESIpos): m/z=351 [M+H]⁺.

### Step 2

### 8-methyl-2-[(4-methylphenyl)methyl]-4,5-dihydro-2H-furo[2,3-g]indazole-7-carboxylic acid

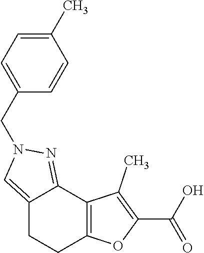 [SMILES: Cc1ccc(Cn2cc3c(n2)-c2c(oc(C(=O)O)c2C)CC3)cc1]

A solution of ethyl 8-methyl-2-[(4-methylphenyl)methyl]-4,5-dihydro-2H-furo[2,3-g]indazole-7-carboxylate (1.00 eq., 409 mg, 1.17 mmol) from step 1 in a 1:1 mixture of ethanol and THF (36 mL) was treated with aqueous lithium hydroxide (1 M; 15 eq., 18 mL, 18 mmol) and stirred at 70° C. overnight. After cooling to rt the reaction mixture was acidified by addition of 4 N aqueous hydrochloric acid (pH 4) and diluted with EtOAc. The layers were separated and the aqueous layer extracted with EtOAc. The combined organic layers were washed with brine, dried with Na₂SO₂, filtrated and concentrated under reduced pressure to give the desired carboxylic acid (364 mg, 92%).

¹H NMR (400 MHz, DMSO-d6) δ [ppm]: 2.27 (s, 3H), 2.46 (s, 3H), 2.83-2.90 (m, 4H), 5.22 (s, 2H), 7.11-7.19 (m, 4H), 7.56 (s, 1H), 12.80 (brs, 1H).

UPLC-MS (Method 1): Rₜ=0.68 min; MS (ESIpos): m/z=323 [M+H]⁺.

### Intermediate 2:

### Step 1

### 3-methyl-N-[(1,2-oxazol-3-yl)methyl]-4-oxo-4,5,6,7-tetrahydro-1-benzofuran-2-carboxamide

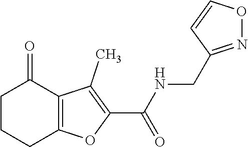 [SMILES: Cc1c(C(=O)NCc2ccon2)oc2c1C(=O)CCC2]

A solution of 3-methyl-4-oxo-4,5,6,7-tetrahydro-1-benzofuran-2-carboxylic acid (CAS No. [112579-43-8]; 1.00 eq., 500 mg, 2.57 mmol) in DMF (35 mL) was treated with 1-(1,2-oxazol-3-yl)methanamine hydrochloride (1:1) (CAS No. [1187933-48-7]; 1.5eq., 520 mg, 3.86 mmol), HATU (CAS No. [148893-10-1]; 1.50 eq., 1.47 g, 3.86 mmol) and N,N-diisopropylethylamine (CAS No. [7087-68-5]; 5.0 eq., 2.2 mL, 13 mmol) and stirred at rt overnight. The reaction mixture was quenched with water and diluted with ethyl acetate. The layers were separated and the organic layer washed with saturated aqueous NaHCO₃ and saturated aqueous ammonium chloride, filtered with a hydrophobic filter, concentrated under reduced pressure and the obtained crude product subjected to column chromatography (Si—NH SiO₂, hexane/EtOAc) to give the title compound (402 mg, 56%).

¹H NMR (400 MHz, DMSO-d6) δ [ppm]: 2.09 (quint, 2H), 2.42-2.44 (m, 5H), 2.91 (t, 2H), 4.47 (d, 2H), 6.48 (d, 1H), 8.82 (d, 1H), 8.91 (t, 1H).

UPLC-MS (Method 1): Rₜ=0.77 min; MS (ESIpos): m/z=275 [M+H]⁺.

### Step 2

### (5E/Z)-5-[(dimethylamino)methylidene]-3-methyl-N-[(1,2-oxazol-3-yl)methyl]-4-oxo-4,5,6,7-tetrahydro-1-benzofuran-2-carboxamide

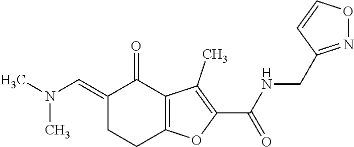 [SMILES: Cc1c(C(=O)NCc2ccon2)oc2c1C(=O)/C(=C/N(C)C)CC2]

3-Methyl-N-[(1,2-oxazol-3-yl)methyl]-4-oxo-4,5,6,7-tetrahydro-1-benzofuran-2-carboxamide (1.00 eq., 320 mg, 1.17 mmol) from step 1 was treated with 1-tert-butoxy-N,N,N′,N′-tetramethylmethanediamine (Bredereck's reagent, CAS No. [5815-08-7]; 2.0 eq., 480 μL, 2.3 mmol) in toluene (2.5 mL) and stirred at 100° C. overnight. The reaction mixture was cooled, concentrated under reduced pressure and the obtained crude title compound (283 mg) used in the subsequent reaction without further purification steps.

UPLC-MS (Method 1): Rₜ=0.82/0.90 min; MS (ESIpos): m/z=330 [M+H]⁺.

### Step 3

### 8-methyl-N-[(1,2-oxazol-3-yl)methyl]-4,5-dihydro-1H-furo[2,3-g]indazole-7-carboxamide

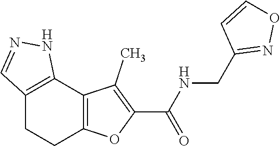 [SMILES: Cc1c(C(=O)NCc2ccon2)oc2c1-c1[nH]ncc1CC2]

The crude (5E/Z)-5-[(dimethylamino)methylidene]-3-methyl-N-[(1,2-oxazol-3-yl)methyl]-4-oxo-4,5,6,7-tetrahydro-1-benzofuran-2-carboxamide (1.0 eq., 280 mg, 860 μmol) from step 2 was treated with a solution of hydrazine hydrate 1:1 (CAS No. [7803-57-8]; 3.0 eq., 120 μL, 2.6 mmol) in ethanol (2 mL) and stirred at 70° C. for 5 hours. The reaction mixture was quenched with sodium hypochlorite at 0° and the biphasic mixture concentrated under reduced pressure. The obtained residue was directly subjected to column chromatography (SiO₂, hexane/EtOAc) to give the title compound (100 mg, 29% over two steps).

UPLC-MS (Method 1): Rₜ=0.76 min; MS (ESIpos): m/z=299 [M+H]⁺.

### Intermediate 3:

### Step 1

### ethyl 8-methyl-2-[(2-methylphenyl)methyl]-4,5-dihydro-2H-furo[2,3-g]indazole-7-carboxylate

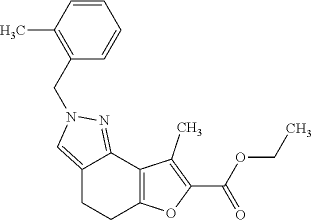 [SMILES: CCOC(=O)c1oc2c(c1C)-c1nn(Cc3ccccc3C)cc1CC2]

A solution of ethyl 8-methyl-4,5-dihydro-1H-furo[2,3-g]indazole-7-carboxylate (commercially available; 1.00 eq., 500 mg, 2.03 mmol; CAS-RN:[903163-04-2]) in ethyl acetate (15 mL) was treated with 1-(bromomethyl)-2-methylbenzene (1.5 eq., 410 μL, 3.0 mmol; CAS-RN:[89-92-9]) and potassium carbonate (15.0 eq., 4.21 g, 30.5 mmol; CAS-RN:[584-08-7]) and stirred at 75° C. overnight. As the conversion was not complete another amount of 1-(bromomethyl)-2-methylbenzene (0.50 eq., 140 μL, 1.0 mmol) and N,N-dimethylpyridin-4-amine (DMAP, 5 mol %, 12 mg, 100 μmol; CAS-RN:[1122-58-3]) was added and stirring at 75° C. continued for another 24 hours. The reaction mixture was cooled to rt and filtrated. The filtrate was concentrated under reduced pressure and the residue subjected to column chromatography (SiO₂, hexane/ethyl acetate) to give the title compound (472 mg, 60%).

¹H NMR (400 MHz, DMSO-d6) δ [ppm]: 1.29 (t, 3H), 2.35 (s, 3H), 2.48 (s, 3H), 2.83-2.94 (m, 4H), 4.26 (q, 2H), 5.29 (s, 2H), 6.92 (d, 1H), 7.11-7.20 (m, 3H), 7.51 (s, 1H).

UPLC-MS (Method 1): Rₜ=1.45 min; MS (ESIpos): m/z=351 [M+H]⁺.

### Step 2

### 8-methyl-2-[(2-methylphenyl)methyl]-4,5-dihydro-2H-furo[2,3-g]indazole-7-carboxylic acid

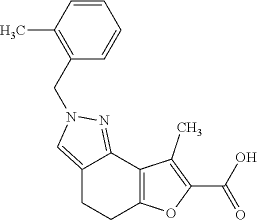 [SMILES: Cc1ccccc1Cn1cc2c(n1)-c1c(oc(C(=O)O)c1C)CC2]

A solution of ethyl 8-methyl-2-[(2-methylphenyl)methyl]-4,5-dihydro-2H-furo[2,3-g]indazole-7-carboxylate (1.00 eq., 462 mg, 1.32 mmol) from step 1 in a 1:1 mixture of ethanol and THF (30 mL) was treated with aqueous lithium hydroxide (1 M; 15 eq., 20 mL, 20 mmol) and stirred at 70° C. overnight. After cooling to rt the reaction mixture was acidified by addition of 4 N aqueous hydrochloric acid (pH 4) and diluted with EtOAc. The layers were separated and the aqueous layer extracted with EtOAc. The combined organic layers were washed with brine, dried with Na₂SO₂, filtrated and concentrated under reduced pressure to give the desired carboxylic acid (401 mg, 85%).

¹H NMR (400 MHz, DMSO-d6) δ [ppm]: 2.35 (s, 3H), 2.46 (s, 3H), 2.82-2.92 (m, 4H), 5.29 (s, 2H), 6.93 (d, 1H), 7.15-7.20 (m, 3H), 7.50 (s, 1H), 12.83 (brs, 1H).

UPLC-MS (Method 1): Rₜ=0.67 min; MS (ESIpos): m/z=323 [M+H]⁺.

### Intermediate 4:

### Step 1

### ethyl 2-[(2-chlorophenyl)methyl]-8-methyl-4,5-dihydro-2H-furo[2,3-g]indazole-7-carboxylate

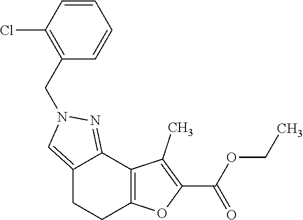 [SMILES: CCOC(=O)c1oc2c(c1C)-c1nn(Cc3ccccc3Cl)cc1CC2]

A solution of ethyl 8-methyl-4,5-dihydro-1H-furo[2,3-g]indazole-7-carboxylate (commercially available; 1.00 eq., 243 mg, 987 μmol; CAS-RN:[903163-04-2]) in acetonitrile (12 mL) was treated with 1-(bromomethyl)-2-chlorobenzene (1.5 eq., 190 μL, 1.5 mmol; CAS-RN:[611-17-6]) and potassium carbonate (15.0 eq., 2.05 g, 14.8 mmol; CAS-RN:[584-08-7]) and stirred at 60° C. overnight. The reaction mixture was cooled to rt and filtrated. The filtrate was concentrated under reduced pressure and the residue subjected to column chromatography (Si—NH SiO₂, hexane/ethyl acetate) to give the title compound (298 mg, 73%).

¹H NMR (400 MHz, DMSO-d6) δ [ppm]: 1.29 (t, 3H), 2.47 (s, 3H), 2.85-2.95 (m, 4H), 4.26 (q, 2H), 5.40 (s, 2H), 6.95-6.98 (m, 1H), 7.30-7.37 (m, 2H), 7.48-7.51 (m, 1H), 7.61 (s, 1H).

UPLC-MS (Method 1): Rₜ=1.46 min; MS (ESIpos): m/z=371/373 [M+H]⁺ (CI isotope pattern).

### Step 2

### 2-[(2-chlorophenyl)methyl]-8-methyl-4,5-dihydro-2H-furo[2,3-g]indazole-7-carboxylic acid

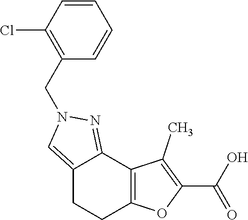 [SMILES: Cc1c(C(=O)O)oc2c1-c1nn(Cc3ccccc3Cl)cc1CC2]

A solution of ethyl 2-[(2-chlorophenyl)methyl]-8-methyl-4,5-dihydro-2H-furo[2,3-g]indazole-7-carboxylate (1.00 eq., 233 mg, 628 μmol) from step 1 in THF (3 mL) was treated with aqueous sodium hydroxide (CAS-RN:[1310-73-2]; 4 M; 30 eq., 4.7 mL, 19 mmol) and stirred at 70° C. for three days. After cooling to rt the reaction mixture was acidified by addition of 8 N aqueous hydrochloric acid (pH 2) and diluted with EtOAc and brine. The layers were separated and the aqueous layer extracted with EtOAc. The combined organic layers were washed with brine, dried with Na₂SO₂, filtrated and concentrated under reduced pressure to give the desired carboxylic acid (198 mg, 84%).

¹H NMR (400 MHz, DMSO-d6) δ [ppm]: 2.45 (s, 3H), 2.84-2.93 (m, 4H), 5.39 (s, 2H), 6.96-6.98 (m, 1H), 7.30-7.36 (m, 2H), 7.48-7.51 (m, 1H), 7.61 (s, 1H), 12.80 (brs, 1H).

UPLC-MS (Method 1): Rₜ=0.66 min; MS (ESIpos): m/z=343/345 [M+H]⁺ (CI isotope pattern).

### Intermediate 5:

### Step 1

### ethyl 2-[(3-chlorophenyl)methyl]-8-methyl-4,5-dihydro-2H-furo[2,3-g]indazole-7-carboxylate

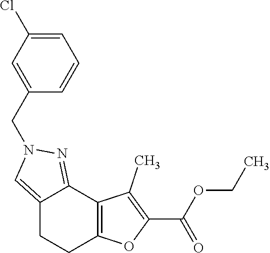 [SMILES: CCOC(=O)c1oc2c(c1C)-c1nn(Cc3cccc(Cl)c3)cc1CC2]

A solution of ethyl 8-methyl-4,5-dihydro-1H-furo[2,3-g]indazole-7-carboxylate (commercially available; 1.00 eq., 241 mg, 979 μmol; CAS-RN:[903163-04-2]) in acetonitrile (12 mL) was treated with 1-(bromomethyl)-3-chlorobenzene (1.5 eq., 190 μL, 1.5 mmol; CAS-RN:[108-37-2]) and potassium carbonate (15.0 eq., 2.03 g, 14.7 mmol; CAS-RN:[584-08-7]) and stirred at 60° C. overnight. The reaction mixture was cooled to rt and filtrated. The filtrate was concentrated under reduced pressure and the residue subjected to column chromatography (Si—NH SiO₂, hexane/ethyl acetate) to give the title compound (253 mg, 66%).

¹H NMR (400 MHz, DMSO-d6) δ [ppm]: 1.29 (t, 3H), 2.48 (s, 3H), 2.84-2.94 (m, 4H), 4.26 (q, 2H), 5.31 (s, 2H), 7.19-7.21 (m, 1H), 7.32-7.40 (m, 3H), 7.65 (s, 1H).

UPLC-MS (Method 1): Rₜ=1.44 min; MS (ESIpos): m/z=371/373 [M+H]⁺ (CI isotope pattern).

### Step 2

### 2-[(3-chlorophenyl)methyl]-8-methyl-4,5-dihydro-2H-furo[2,3-g]indazole-7-carboxylic acid

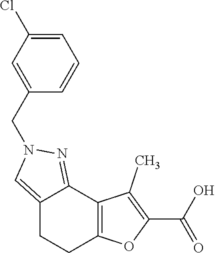 [SMILES: Cc1c(C(=O)O)oc2c1-c1nn(Cc3cccc(Cl)c3)cc1CC2]

A solution of ethyl 2-[(3-chlorophenyl)methyl]-8-methyl-4,5-dihydro-2H-furo[2,3-g]indazole-7-carboxylate (1.00 eq., 193 mg, 520 μmol) from step 1 in THF (2.5 mL) was treated with aqueous sodium hydroxide (CAS-RN:[1310-73-2]; 4 M; 30 eq., 3.9 mL, 16 mmol) and stirred at 70° C. for three days. After cooling to rt the reaction mixture was acidified by addition of 8 N aqueous hydrochloric acid (pH 2) and diluted with EtOAc and brine. The layers were separated and the aqueous layer extracted with EtOAc. The combined organic layers were washed with brine, dried with Na₂SO₂, filtrated and concentrated under reduced pressure to give the desired carboxylic acid (139 mg, 70%).

¹H NMR (400 MHz, DMSO-d6) δ [ppm]: 2.48 (s, 3H), 2.83-2.92 (m, 4H), 5.31 (s, 2H), 7.19-7.21 (m, 1H), 7.31-7.40 (m, 3H), 7.65 (s, 1H), 12.80 (brs, 1H).

UPLC-MS (Method 1): Rₜ=0.65 min; MS (ESIpos): m/z=343/345 [M+H]⁺ (CI isotope pattern).

### Intermediate 6:

### Step 1

### ethyl 2-[(4-chlorophenyl)methyl]-8-methyl-4,5-dihydro-2H-furo[2,3-g]indazole-7-carboxylate

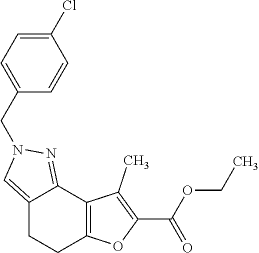 [SMILES: CCOC(=O)c1oc2c(c1C)-c1nn(Cc3ccc(Cl)cc3)cc1CC2]

A solution of ethyl 8-methyl-4,5-dihydro-1H-furo[2,3-g]indazole-7-carboxylate (commercially available; 1.00 eq., 640 mg, 2.60 mmol; CAS-RN:[903163-04-2]) in acetonitrile (25 mL) was treated with 1-(bromomethyl)-4-chlorobenzene (1.5 eq., 800 mg, 3.9 mmol; CAS-RN:[622-95-7]) and potassium carbonate (10 eq., 3.6 g, 26 mmol; CAS-RN:[584-08-7]) and stirred at 60° C. overnight. The reaction mixture was cooled to rt and filtrated. The filtrate was concentrated under reduced pressure to give the crude title compound (1.0 g, 93%) which was used in the next step without further purification.

¹H NMR (400 MHz, DMSO-d6) δ [ppm]: 1.29 (t, 3H), 2.47 (s, 3H), 2.83-2.93 (m, 4H), 4.26 (q, 2H), 5.30 (s, 2H), 7.24-7.27 (m, 2H), 7.40-7.43 (m, 2H), 7.63 (s, 1H).

UPLC-MS (Method 1): Rₜ=1.44 min; MS (ESIpos): m/z=371/373 [M+H]⁺ (CI isotope pattern).

### Step 2

### 2-[(4-chlorophenyl)methyl]-8-methyl-4,5-dihydro-2H-furo[2,3-g]indazole-7-carboxylic acid

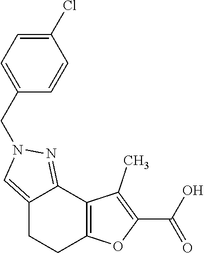 [SMILES: Cc1c(C(=O)O)oc2c1-c1nn(Cc3ccc(Cl)cc3)cc1CC2]

A solution of ethyl 2-[(4-chlorophenyl)methyl]-8-methyl-4,5-dihydro-2H-furo[2,3-g]indazole-7-carboxylate (1.00 eq., 1.00 g, 2.43 mmol) from step 1 in THF (9 mL) was treated with aqueous sodium hydroxide (CAS-RN:[1310-73-2]; 4 M; 30 eq., 18 mL, 73 mmol) and stirred at 70° C. overnight. After cooling to rt the reaction mixture was acidified by addition of 6 N aqueous hydrochloric acid (pH 2) and diluted with EtOAc and brine. The layers were separated and the aqueous layer extracted several times with EtOAc. The combined organic layers were washed with brine, dried with Na₂SO₂, filtrated and concentrated under reduced pressure to give the desired carboxylic acid (790 mg, 84%).

¹H NMR (400 MHz, DMSO-d6) δ [ppm]: 2.45 (s, 3H), 2.82-2.91 (m, 4H), 5.29 (s, 2H), 7.24-7.27 (m, 2H), 7.40-7.43 (m, 2H), 7.61 (s, 1H), 12.82 (brs, 1H).

UPLC-MS (Method 2): Rₜ=1.17 min; MS (ESIpos): m/z=343/345 [M+H]⁺ (CI isotope pattern).

### Intermediate 7:

### Step 1

### ethyl 2-[(2-chloro-4-fluorophenyl)methyl]-8-methyl-4,5-dihydro-2H-furo[2,3-g]indazole-7-carboxylate

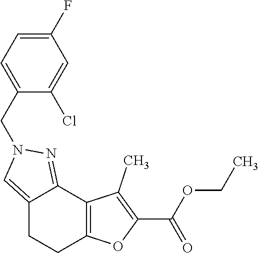 [SMILES: CCOC(=O)c1oc2c(c1C)-c1nn(Cc3ccc(F)cc3Cl)cc1CC2]

A solution of ethyl 8-methyl-4,5-dihydro-1H-furo[2,3-g]indazole-7-carboxylate (commercially available; 1.00 eq., 660 mg, 2.68 mmol; CAS-RN:[903163-04-2]) in acetonitrile (35 mL) was treated with 1-(bromomethyl)-2-chloro-4-fluorobenzene (1.5 eq., 898 mg, 4.0 mmol; CAS-RN:[45767-66-6]) and potassium carbonate (10 eq., 3.7 g, 27 mmol; CAS-RN:[584-08-7]) and stirred at 60° C. overnight. The reaction mixture was cooled to rt and filtrated. The filtrate was concentrated under reduced pressure and the residue subjected to column chromatography (SiO₂, dicholormethane/methanol) to give the title compound (391 mg, 32%).

¹H NMR (400 MHz, DMSO-d6) δ [ppm]: 1.29 (t, 3H), 2.47 (s, 3H), 2.84-2.94 (m, 4H), 4.26 (q, 2H), 5.37 (s, 2H), 7.08 (dd, 1H), 7.23 (dt, 1H), 7.51 (dd, 1H), 7.60 (s, 1H).

UPLC-MS (Method 1): Rₜ=1.48 min; MS (ESIpos): m/z=389/391 [M+H]⁺ (CI isotope pattern).

### Step 2

### 2-[(2-chloro-4-fluorophenyl)methyl]-8-methyl-4,5-dihydro-2H-furo[2,3-g]indazole-7-carboxylic acid

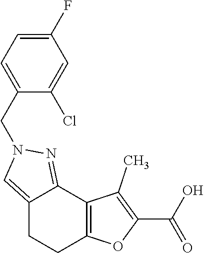 [SMILES: Cc1c(C(=O)O)oc2c1-c1nn(Cc3ccc(F)cc3Cl)cc1CC2]

A solution of ethyl 2-[(2-chloro-4-fluorophenyl)methyl]-8-methyl-4,5-dihydro-2H-furo[2,3-g]indazole-7-carboxylate (1.00 eq., 385 mg, 832 μmol) from step 1 in THF (5 mL) was treated with aqueous sodium hydroxide (CAS-RN:[1310-73-2]; 4 M; 30 eq., 6.2 mL, 25 mmol) and stirred at 70° C. overnight. After cooling to rt the reaction mixture was acidified by addition of 6 N aqueous hydrochloric acid (pH 2) and diluted with EtOAc and brine. The layers were separated and the aqueous layer extracted with EtOAc. The combined organic layers were washed with brine, dried with Na₂SO₂, filtrated and concentrated under reduced pressure to give the desired carboxylic acid (350 mg, 98%).

¹H NMR (400 MHz, DMSO-d6) δ [ppm]: 2.45 (s, 3H), 2.84-2.93 (m, 4H), 5.37 (s, 2H), 7.08 (dd, 1H), 7.23 (dt, 1H), 7.51 (dd, 1H), 7.60 (s, 1H), 12.81 (brs, 1H).

UPLC-MS (Method 1): Rₜ=0.69 min; MS (ESIpos): m/z=361/363 [M+H]⁺ (CI isotope pattern).

### Intermediate 8:

### Step 1

### ethyl 2-[(1RS)-1-(2-fluorophenyl)ethyl]-8-methyl-4,5-dihydro-2H-furo[2,3-g]indazole-7-carboxylate

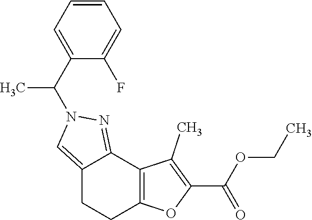 [SMILES: CCOC(=O)c1oc2c(c1C)-c1nn(C(C)c3ccccc3F)cc1CC2]

Ethyl 8-methyl-4,5-dihydro-1H-furo[2,3-g]indazole-7-carboxylate (commercially available; 1.00 eq., 150 mg, 609 μmol; CAS-RN:[903163-04-2]) was reacted with (1RS)-1-(2-fluorophenyl)ethan-1-ol (102 mg, 731 μmol; CAS-RN:[903163-04-2]), tri-n-butylphosphine (240 μl, 970 μmol; CAS-RN:[998-40-3]) and TMAD (168 mg, 975 μmol; CAS-RN:[10465-78-8]) in toluene (5.5 mL) at rt for 48 h. Further tri-n-butylphosphine (150 μL, 606 μmol) and TMAD (105 mg, 609 μmol) were added and stirring was continued for 24 h. The reaction mixture was diluted water while stirring was continued for 30 min. After phase separation, the aqueous layer was extracted with toluene. The combined organic phases were dried with a hydrophobic filter paper and concentrated in vacuo. The residue was purified by Biotage Isolera™ chromatography (SNAP KP-Sil—10 g, eluting with dichloromethane-ethanol, 95:5) to afford 66.6 mg (19% yield, 65% purity) of the title compound.

LC-MS (Method 1): Rt=1.48 min; MS (ESIpos): m/z=369 [M+H]⁺.

### Step 2

### 2-[(1RS)-1-(2-fluorophenyl)ethyl]-8-methyl-4,5-dihydro-2H-furo[2,3-g]indazole-7-carboxylic acid

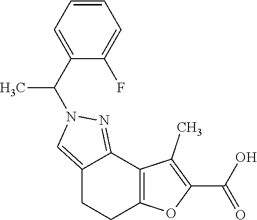 [SMILES: Cc1c(C(=O)O)oc2c1-c1nn(C(C)c3ccccc3F)cc1CC2]

Ethyl 2-[(1RS)-1-(2-fluorophenyl)ethyl]-8-methyl-4,5-dihydro-2H-furo[2,3-g]indazole-7-carboxylate (66.6 mg, 65% purity, 118 μmol) from step 1 was reacted with aqueous lithium hydroxide (1.2 mL, 1.0 M, 1.2 mmol; CAS-RN:[1310-65-2]) in THF (150 μL) at rt for 6 days. The reaction mixture was acidified with aqueous 4 N HCl (pH 2) and concentrated in vacuo to afford 42 mg (crude) of the title compound.

LC-MS (Method 1): Rt=0.66 min; MS (ESIpos): m/z=341 [M+H]⁺.

### EXPERIMENTAL SECTION—EXAMPLES

### Example 1

### 8-methyl-2-[(4-methylphenyl)methyl]-N-[(2S)-tetrahydrofuran-2-ylmethyl]-4,5-dihydro-2H-furo[2,3-g]indazole-7-carboxamide

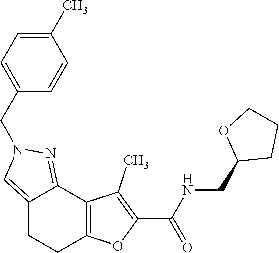 [SMILES: Cc1ccc(Cn2cc3c(n2)-c2c(oc(C(=O)NC[C@@H]4CCCO4)c2C)CC3)cc1]

A solution of 8-methyl-2-[(4-methylphenyl)methyl]-4,5-dihydro-2H-furo[2,3-g]indazole-7-carboxylic acid (Intermediate 1; 1.00 eq., 105 mg, 326 μmol) in DMF (2 mL) was treated with 1-[(2S)-tetrahydrofuran-2-yl]methanamine (CAS No. [7175-81-7]; 1.2 eq., 40 μL, 390 μmol), HATU (CAS No. [148893-10-1]; 1.50 eq., 186 mg, 489 μmol) and N,N-diisopropylethylamine (CAS No. [7087-68-5]; 3.0 eq., 170 μL, 980 μmol) and stirred at rt overnight to give upon preparative HPLC the title compound (96 mg, 72%).

¹H NMR (400 MHz, DMSO-d6) δ [ppm]: 1.53-1.61 (m, 1H), 1.73-1.91 (m, 3H), 2.27 (s, 3H), 2.46 (s, 3H), 2.81-2.90 (m, 4H), 3.18-3.28 (m, 2H), 3.58-3.63 (m, 1H), 3.73-3.78 (m, 1H), 3.91-3.97 (m, 1H), 5.22 (s, 2H), 7.15 (s, 4H), 7.54 (s, 1H) 7.97 (t, 1H).

LC-MS (Method A): Rₜ=1.23 min; MS (ESIpos): m/z=406 [M+H]⁺.

### Example 2

### N-{[(2R)-1,4-dioxan-2-yl]methyl}-8-methyl-2-[(4-methylphenyl)methyl]-4,5-dihydro-2H-furo[2,3-g]indazole-7-carboxamide

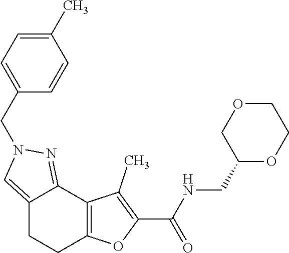 [SMILES: Cc1ccc(Cn2cc3c(n2)-c2c(oc(C(=O)NC[C@@H]4COCCO4)c2C)CC3)cc1]

Example 2 was prepared in analogy to Example 1 starting from 8-methyl-2-[(4-methylphenyl)methyl]-4,5-dihydro-2H-furo[2,3-g]indazole-7-carboxylic acid (Intermediate 1; 1.00 eq., 99 mg, 307 μmol) and 1-[(2R)-1,4-dioxan-2-yl]methanamine hydrochloride (1:1) (CAS No. [1523541-84-5]; 1.2 eq., 57 mg, 370 μmol) yielding 60 mg (44%) of the title compound.

¹H NMR (400 MHz, DMSO-d6) δ [ppm]: 2.27 (s, 3H), 2.46 (s, 3H), 2.76-2.91 (m, 4H), 3.14-3.28 (m, 2H), 3.41-3.76 (m, 7H), 5.22 (s, 2H), 7.15 (s, 4H), 7.54 (s, 1H) 8.05 (t, 1H).

LC-MS (Method A): Rₜ=1.18 min; MS (ESIpos): m/z=422 [M+H]⁺.

### Example 3

### 8-methyl-2-[(4-methylphenyl)methyl]-N-[(1,2-oxazol-3-yl)methyl]-4,5-dihydro-2H-furo[2,3-g]indazole-7-carboxamide

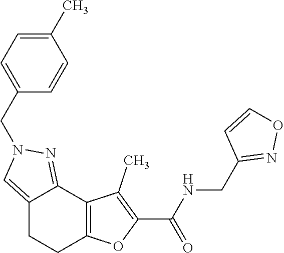 [SMILES: Cc1ccc(Cn2cc3c(n2)-c2c(oc(C(=O)NCc4ccon4)c2C)CC3)cc1]

A solution of 8-methyl-N-[(1,2-oxazol-3-yl)methyl]-4,5-dihydro-1H-furo[2,3-g]indazole-7-carboxamide (Intermediate 2; 1.00 eq., 50.0 mg, 168 μmol) in acetonitrile (2 mL) was treated with 1-(bromomethyl)-4-methylbenzene (1.5 eq., 47 mg, 250 μmol; CAS-RN:[104-81-4]) and potassium carbonate (15.0 eq., 347 mg, 2.51 mmol; CAS-RN:[584-08-7]) and stirred at 60° C. overnight. The reaction mixture was cooled to rt and filtrated. The filtrate was concentrated under reduced pressure and the residue subjected to reparative HPLC to give the title compound (0.7 mg, 1%).

LC-MS (Method A): Rₜ=1.22 min; MS (ESIpos): m/z=403 [M+H]⁺.

### Example 4

### 8-methyl-2-[(2-methylphenyl)methyl]-N-[(2S)-tetrahydrofuran-2-ylmethyl]-4,5-dihydro-2H-furo[2,3-g]indazole-7-carboxamide

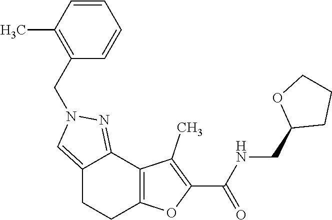 [SMILES: Cc1ccccc1Cn1cc2c(n1)-c1c(oc(C(=O)NC[C@@H]3CCCO3)c1C)CC2]

A solution of 8-methyl-2-[(2-methylphenyl)methyl]-4,5-dihydro-2H-furo[2,3-g]indazole-7-carboxylic acid (Intermediate 3; 1.00 eq., 105 mg, 326 μmol) in DMF (2 mL) was treated with 1-[(2S)-tetrahydrofuran-2-yl]methanamine (CAS No. [7175-81-7]; 1.2 eq., 40 μL, 390 μmol), HATU (CAS No. [148893-10-1]; 1.50 eq., 186 mg, 489 μmol) and N,N-diisopropylethylamine (CAS No. [7087-68-5]; 3.0 eq., 170 μL, 980 μmol) and stirred at rt overnight to give upon preparative HPLC the title compound (85 mg, 59%).

¹H NMR (400 MHz, DMSO-d6) δ [ppm]: 1.53-1.63 (m, 1H), 1.76-1.91 (m, 3H), 2.35 (s, 3H), 2.45 (s, 3H), 2.83-2.92 (m, 4H), 3.18-3.26 (m, 2H), 3.58-3.64 (m, 1H), 3.73-3.78 (m, 1H), 3.91-3.97 (m, 1H), 5.28 (s, 2H), 6.93 (d, 1H), 7.13-7.20 (m, 3H), 7.48 (s, 1H) 7.98 (t, 1H).

LC-MS (Method A): Rₜ=1.24 min; MS (ESIpos): m/z=406 [M+H]⁺.

TABLE 2
The following examples (5 to 7) were prepared in analogy to Example 4
starting from the given intermediates and commercially available amines (or their
salts), or were prepared applying the indicated procedure.

| | | | Preparation |
|---|---|---|---|
| | Structure | | or |
| | IUPAC- | | Separation |
| Example | Name | Analytical Data | Methods |
| 5 | 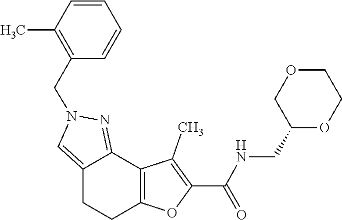 [SMILES: Cc1ccccc1Cn1cc2c(n1)-c1c(oc(C(=O)NC[C@@H]3COCCO3)c1C)CC2] | ¹H NMR (400 MHz, DMSO-d6) δ [ppm]: 2.35-2.45 (m, 6H), 2.76- 2.92 (m, 4H), 3.15-3.26 (m, 3H), 3.41-3.76 (m, 6H), 5.28 (s, 1.5H), 5.71 (s, 0.5H), 6.93 (d, 0.75H), 6.99 (d, 0.25H), 7.14-7.24 (m, 3H), 7.48 (s, 0.75H), 8.06 (t, 0.75H), 8.42 (t, 0.25H), 8.46 (s, 0.25H). LC-MS (Method A); Rₜ = 1.19 min, m/z = 422 [M + H]⁺. | Intermediate 3 and CAS-RN: [1523541-84-5]; (conditions with HATU) 67 mg (35% yield) |
| | N-{[(2R)-1,4-dioxan-2-yl]methyl}-8- | | |
| | methyl-2-[(2-methylphenyl)methyl]- | | |
| | 4,5-dihydro-2H-furo[2,3-g]indazole- | | |
| | 7-carboxamide | | |
| 6 | 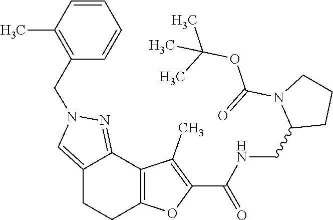 [SMILES: Cc1ccccc1Cn1cc2c(n1)-c1c(oc(C(=O)NCC3CCCN3C(=O)OC(C)(C)C)c1C)CC2] | ¹H NMR (400 MHz, DMSO-d6) δ [ppm]: 1.39 (brs, 9H), 1.78-1.83 (m, 4H), 2.35 (s, 3H), 2.45 (s, 3H), 2.86-2.89 (m, 4H), 3.09- 3.25 (m, 4H), 3.91-3.95 (m, 1H), 5.28 (s, 2H), 6.93 (d, 1H), 7.13- 7.20 (m, 3H), 7.48 (s, 1H), 8.09- 8.16 (m, 1H). LC-MS (Method A); Rₜ = 1.46 min, m/z = 505 [M + H]⁺. | Intermediate 3 and CAS-RN: [177911-87-4]; (conditions with HATU) 92 mg (41% yield) |
| | tert-butyl (2RS)-2-[{{8-methyl-2-[(2- | | |
| | methylphenyl)methyl]-4,5-dihydro- | | |
| | 2H-furo[2,3-g]indazole-7- | | |
| | carbonyl}amino)methyl]pyrrolidine- | | |
| | 1-carboxylate | | |
| 7 | 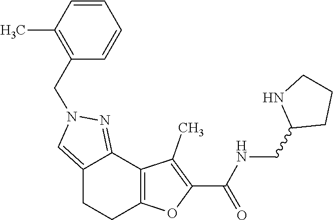 [SMILES: Cc1ccccc1Cn1cc2c(n1)-c1c(oc(C(=O)NCC3CCCN3)c1C)CC2] | ¹H NMR (400 MHz, DMSO-d6) δ [ppm]: 1.34-1.39 (m, 1H), 1.54- 1.85 (m, 3H), 2.35-2.45 (m, 6H), 2.69-2.91 (m, 5H), 3.04-3.29 (m, 4H), 5.28 (s, 1.6H), 5.71 (s, 0.4H), 6.93 (d, 0.8H), 6.99 (d, 0.2H), 7.13-7.24 (m, 3H), 7.48 (s, 0.8H), 7.88 (t, 0.8H), 8.23 (t, 0.2H), 8.46 (s, 0.2H). LC-MS (Method A); Rₜ = 1.24 min, m/z = 405 [M + H]⁺. | Prepared by hydrolysis of Example 6 with HCl in CPME (3 M) at rt followed by preparative HPLC: 34 mg (45% yield). |
| | 8-methyl-2-[(2- | | |
| | methylphenyl)methyl]-N-{[(2RS)- | | |
| | pyrrolidin-2-yl]methyl}-4,5-dihydro- | | |
| | 2H-furo[2,3-g]indazole-7- | | |
| | carboxamide | | |
| 7-1 | Enantiomer 1 of Example 7 | Rₜ = 6.81 min. | analyt. method: |
| | Chiral separation of PAAN4608-1 | | Instrument: |
| | | | Agilent: 1260, |
| | | | Aurora SFC- |
| | | | Modul; |
| | | | Column: |
| | | | Chiralpak IG |
| | | | 5 μm, |
| | | | 100 × 4.6 mm; |
| | | | Eluent A: CO₂; |
| | | | Eluent B: |
| | | | ethanol + |
| | | | 0.2 vol % |
| | | | aqueous |
| | | | ammonia |
| | | | (32%); |
| | | | Isocratic: |
| | | | 25% B; Flow: |
| | | | 4 mL/min; |
| | | | Temperature: |
| | | | 37.5° C.; BPR: |
| | | | 100 bar; UV: |
| | | | 254 nm. |
| 7-2 | Enantiomer 2 of Example 7 | Rₜ = 8.52 min. | |
| | Chiral separation of PAAN4608-1 | | |
| 7-3 | 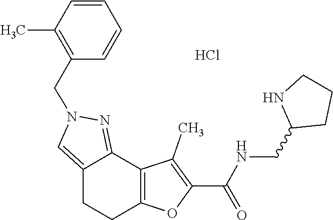 [SMILES: Cc1ccccc1Cn1cc2c(n1)-c1c(oc(C(=O)NCC3CCCN3)c1C)CC2 ; Cl] | UPLC-MS (Method 1); Rₜ = 1.23 min, m/z = 405 [M + H]⁺ | Prepared by hydrolysis of Example 6 with HCl in CPME (3 M) at rt followed by concentration in vacuo: 67 mg (81% yield). |
| | 8-methyl-2-[(2- | | |
| | methylphenyl)methyl]-N-{[(2RS)- | | |
| | pyrrolidin-2-yl]methyl}-4,5-dihydro- | | |
| | 2H-furo[2,3-g]indazole-7- | | |
| | carboxamide hydrochloride (1:1) | | |

### Example 8

### 8-methyl-2-[(2-methylphenyl)methyl]-N-[(1,2-oxazol-3-yl)methyl]-4,5-dihydro-2H-furo[2,3-g]indazole-7-carboxamide

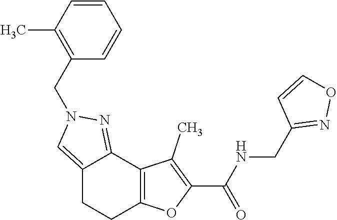 [SMILES: Cc1ccccc1Cn1cc2c(n1)-c1c(oc(C(=O)NCc3ccon3)c1C)CC2]

A solution of 8-methyl-N-[(1,2-oxazol-3-yl)methyl]-4,5-dihydro-1H-furo[2,3-g]indazole-7-carboxamide (Intermediate 2; 1.00 eq., 50.0 mg, 168 μmol) in acetonitrile (2 mL) was treated with 1-(bromomethyl)-2-methylbenzene (1.5 eq., 46 mg, 250 μmol; CAS-RN:[89-92-9]) and potassium carbonate (15.0 eq., 347 mg, 2.51 mmol; CAS-RN:[584-08-7]) and stirred at 60° C. overnight. The reaction mixture was cooled to rt and filtrated. The filtrate was concentrated under reduced pressure and the residue subjected to reparative HPLC to give the title compound (1.2 mg, 2%).

LC-MS (Method A): Rₜ=1.22 min; MS (ESIpos): m/z=403 [M+H]⁺.

### Example 9

### 2-[(2-chlorophenyl)methyl]-8-methyl-N-[(2RS)-tetrahydrofuran-2-ylmethyl]-4,5-dihydro-2H-furo[2,3-g]indazole-7-carboxamide

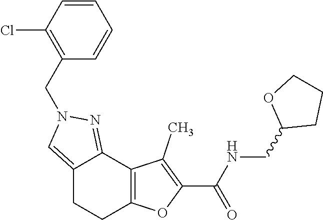 [SMILES: Cc1c(C(=O)NCC2CCCO2)oc2c1-c1nn(Cc3ccccc3Cl)cc1CC2]

A solution of 2-[(2-chlorophenyl)methyl]-8-methyl-4,5-dihydro-2H-furo[2,3-g]indazole-7-carboxylic acid (Intermediate 4; 1.00 eq., 101 mg, 295 μmol) in DMF (1 mL) was treated with 1-[(2RS)-tetrahydrofuran-2-yl]methanamine (CAS No. [4795-29-3]; 1.2 eq., 36 μL, 350 μmol), HATU (CAS No. [148893-10-1]; 1.50 eq., 168 mg, 442 μmol) and N,N-diisopropylethylamine (CAS No. [7087-68-5]; 3.0 eq., 150 μL, 880 μmol) and stirred at rt overnight. The reaction mixture was diluted with EtOAc and the layers separated. The organic layer was washed with water, dried with Na₂SO₂, filtrated and concentrated under reduced pressure. The obtained residue was subjected to column chromatography (Si—NH SiO₂, hexane/ethyl acetate) to give the title compound (96 mg, 73%).

¹H NMR (400 MHz, DMSO-d6) δ [ppm]: 1.53-1.59 (m, 1H), 1.76-1.90 (m, 3H), 2.44 (s, 3H), 2.88-2.90 (m, 4H), 3.21-3.25 (m, 2H), 3.58-3.64 (m, 1H), 3.73-3.78 (m, 1H), 3.91-3.97 (m, 1H), 5.39 (s, 2H), 6.96-6.98 (m, 1H), 7.30-7.37 (m, 2H), 7.47-7.52 (m, 1H), 7.59 (s, 1H), 7.99 (t, 1H).

UPLC-MS (Method 2): Rₜ=1.28 min; MS (ESIpos): m/z=426/428 [M+H]⁺ (CI isotope pattern).

The enantiomers of the racemic material of example 9 were separated by chiral preparative HPLC (Instrument Sepiatec: Prep SFC100; Column: Chiralpak IA 5 μm 250×30 mm; Eluent A: CO₂; Eluent B: methanol; Isocratic: 30% B; Flow: 100 mL/min; Temperature: 40° C.; BPR: 150 bar; Detection: UV 220 nm) and analytically characterized by chiral HPLC (Instrument: Agilent 1260, Aurora SFC-Modul; Column: Chiralpak IA 5 μm 100×4.6 mm; Eluent A: CO₂; Eluent B: methanol; Isocratic: 30% B; Flow: 4 mL/min; Temperature: 37° C.; BPR: 100 bar; Detection: UV: 220 nm):

### Example 9-1

15 mg; Rₜ=2.02 min.

### Example 9-2

15 mg; Rₜ=2.38 min.

TABLE 3
The following examples (10 to 14) were prepared in analogy to Example 9
starting from Intermediate 4 and commercially available amines (or their salts).

| | Structure | | |
|---|---|---|---|
| | IUPAC- | | Preparation |
| Example | Name | Analytical Data | Methods |
| 10 | 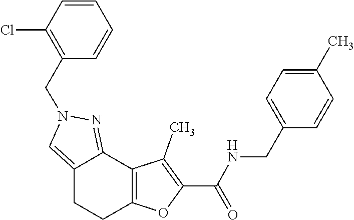 [SMILES: Cc1ccc(CNC(=O)c2oc3c(c2C)-c2nn(Cc4ccccc4Cl)cc2CC3)cc1] | ¹H NMR (400 MHz, DMSO-d6) δ [ppm]: 2.26 (s, 3H), 2.45 (s, 3H), 2.88-2.90 (m, 4H), 4.33 (d, 2H), 5.39 (s, 2H), 6.94-6.99 (m, 1H), 7.10-7.12 (m, 2H), 7.17-7.19 (m, 2H), 7.30-7.36 (m, 2H), 7.47-7.52 (m, 1H), 7.59 (s, 1H), 8.63 (t, 1H). LC-MS (Method A); Rₜ = 1.45 min, m/z = 446/448 [M + H]⁺ (Cl isotope pattern). | Intermediate 4 and CAS-RN: [104-84-7]; (conditions with HATU) 50 mg (49% yield) |
| | 2-[(2-chlorophenyl)methyl]-8-methyl- | | |
| | N-[(4-methylphenyl)methyl]-4,5- | | |
| | dihydro-2H-furo[2,3-g]indazole-7- | | |
| | carboxamide | | |
| 11 | 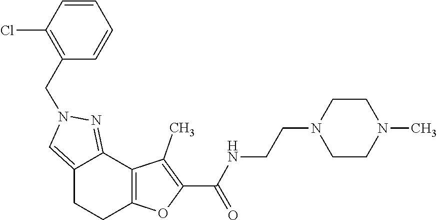 [SMILES: Cc1c(C(=O)NCCN2CCN(C)CC2)oc2c1-c1nn(Cc3ccccc3Cl)cc1CC2] | ¹H NMR (400 MHz, DMSO-d6) δ [ppm]: 2.13 (s, 3H), 2.28-2.44 (m, 13H), 2.84-2.93 (m, 4H), 3.27- 3.32 (m, 2H), 5.39 (s, 2H), 6.95- 6.99 (m, 1H), 7.30-7.37 (m, 2H), 7.47-7.52 (m, 1H), 7.59 (s, 1H), 7.93 (t, 1H). LC-MS (Method A); Rₜ = 1.15 min, m/z = 468/470 [M + H]⁺ (Cl isotope pattern). | Intermediate 4 and CAS-RN: [934-98-5]; (conditions with HATU) 53 mg (51% yield) |
| | 2-[(2-chlorophenyl)methyl]-8-methyl- | | |
| | N-[2-(4-methylpiperazin-1-yl)ethyl]- | | |
| | 4,5-dihydro-2H-furo[2,3-g]indazole- | | |
| | 7-carboxamide | | |
| 12 | 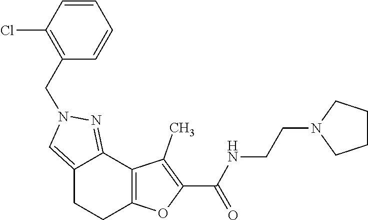 [SMILES: Cc1c(C(=O)NCCN2CCCC2)oc2c1-c1nn(Cc3ccccc3Cl)cc1CC2] | ¹H NMR (400 MHz, DMSO-d6) δ [ppm]: 1.65-1.68 (m, 4H), 2.44- 2.47 (m, 7H), 2.84-2.93 (m, 4H), 3.28-3.32 (m, 4H), 5.39 (s, 2H), 6.96-6.98 (m, 1H), 7.30-7.36 (m, 2H), 7.48-7.51 (m, 1H), 7.59 (s, 1H), 7.95 (t, 1H). LC-MS (Method A); Rₜ = 1.32 min, m/z = 439/441 [M + H]⁺ (Cl isotope pattern). | Intermediate 4 and CAS-RN: [7154-73-6]; (conditions with HATU) 14 mg (13% yield) |
| | 2-[(2-chlorophenyl)methyl]-8-methyl- | | |
| | N-[2-(pyrrolidin-1-yl)ethyl]-4,5- | | |
| | dihydro-2H-furo[2,3-g]indazole-7- | | |
| | carboxamide | | |
| 13 | 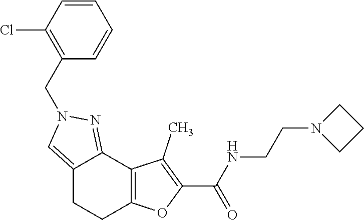 | ¹H NMR (400 MHz, DMSO-d6) δ [ppm]: 1.35-1.51 (m, 6H), 2.32- 2.39 (m, 6H), 2.44 (s, 3H), 2.88- 2.90 (m, 4H), 3.26-3.31 (m, 2H), 5.39 (s, 2H), 6.96-6.98 (m, 1H), 7.30-7.37 (m, 2H), 7.48-7.51 (m, 1H), 7.59 (s, 1H), 7.91 (t, 1H). LC-MS (Method A); Rₜ = 1.40 min, m/z = 453/455 [M + H]⁺ (Cl isotope pattern). | Intermediate 4 and CAS-RN: [27578-60-5]; (conditions with HATU) 50 mg (50% yield) |
| | 2-[(2-chlorophenyl)methyl]-8-methyl- | | |
| | N-[2-(piperidin-1-yl)ethyl]-4,5- | | |
| | dihydro-2H-furo[2,3-g]indazole-7- | | |
| | carboxamide | | |
| 14 | 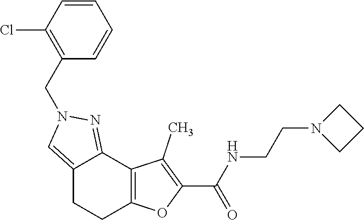 [SMILES: Cc1c(C(=O)NCCN2CCC2)oc2c1-c1nn(Cc3ccccc3Cl)cc1CC2] | ¹H NMR (400 MHz, DMSO-d6) δ [ppm]: 1.94 (quint, 2H), 2.42-2.45 (m, 5H), 2.86-2.92 (m, 4H), 3.08- 3.14 (m, 6H), 5.39 (s, 2H), 6.96- 6.98 (m, 1H), 7.30-7.37 (m, 2H), 7.48-7.52 (m, 1H), 7.59 (s, 1H), 7.88 (t, 1H). LC-MS (Method A); Rₜ = 1.26 min, m/z = 425/427 [M + H]⁺ (Cl isotope pattern). | Intermediate 4 and CAS-RN: [795299-77- 3]; (conditions with HATU) 5 mg (5% yield) |
| | N-[2-(azetidin-1-yl)ethyl]-2-[(2- | | |
| | chlorophenyl)methyl]-8-methyl-4,5- | | |
| | dihydro-2H-furo[2,3-g]indazole-7- | | |
| | carboxamide | | |

### Example 15

### 2-[(3-chlorophenyl)methyl]-8-methyl-N-[(2RS)-tetrahydrofuran-2-ylmethyl]-4,5-dihydro-2H-furo[2,3-g]indazole-7-carboxamide

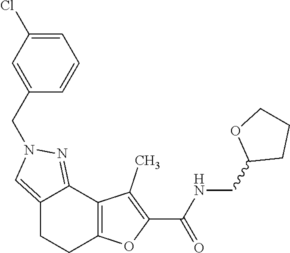 [SMILES: Cc1c(C(=O)NCC2CCCO2)oc2c1-c1nn(Cc3cccc(Cl)c3)cc1CC2]

A solution of 2-[(3-chlorophenyl)methyl]-8-methyl-4,5-dihydro-2H-furo[2,3-g]indazole-7-carboxylic acid (Intermediate 5; 1.00 eq., 101 mg, 295 μmol) in DMF (1 mL) was treated with 1-[(2RS)-tetrahydrofuran-2-yl]methanamine (CAS No. [4795-29-3]; 1.2 eq., 36 μL, 350 μmol), HATU (CAS No. [148893-10-1]; 1.50 eq., 168 mg, 442 μmol) and N,N-diisopropylethylamine (CAS No. [7087-68-5]; 3.0 eq., 150 μL, 880 μmol) and stirred at rt overnight. The reaction mixture was diluted with EtOAc and the layers separated. The organic layer was washed with water, dried with Na₂SO₂, filtrated and concentrated under reduced pressure. The obtained residue was subjected to column chromatography (Si—NH SiO₂, hexane/ethyl acetate) to give the title compound (80 mg, 60%).

¹H NMR (400 MHz, DMSO-d6) δ [ppm]: 1.53-1.61 (m, 1H), 1.73-1.91 (m, 3H), 2.46 (s, 3H), 2.83-2.92 (m, 4H), 3.21-3.25 (m, 2H), 3.58-3.64 (m, 1H), 3.73-3.78 (m, 1H), 3.91-3.97 (m, 1H), 5.30 (s, 2H), 7.19-7.21 (m, 1H), 7.31-7.32 (m, 1H), 7.34-7.40 (m, 2H), 7.63 (s, 1H), 7.98 (t, 1H).

UPLC-MS (Method 2): Rₜ=1.26 min; MS (ESIpos): m/z=426/428 [M+H]⁺ (CI isotope pattern).

The enantiomers of the racemic material of Example 15 were separated by chiral preparative HPLC (Instrument Sepiatec: Prep SFC100; Column: Chiralpak IA 5 μm 250×30 mm; Eluent A: CO₂; Eluent B: methanol; Isocratic: 33% B; Flow: 100 mL/min; Temperature: 40° C.; BPR: 150 bar; Detection: UV 220 nm) and analytically characterized by chiral HPLC (Instrument: Agilent 1260, Aurora SFC-Modul; Column: Chiralpak IA 5 μm 100×4.6 mm; Eluent A: CO₂; Eluent B: methanol; Isocratic: 33% B; Flow: 4 mL/min; Temperature: 37° C.; BPR: 100 bar; Detection: UV: 220 nm):

### Example 15-1

18 mg; Rₜ=2.03 min.

### Example 15-2

20 mg; Rₜ=2.62 min.

TABLE 4
The following examples (16 to 20) were prepared in analogy to Example
15 starting from Intermediate 5 and commercially available amines (or their salts).

| | Structure | | |
|---|---|---|---|
| | IUPAC- | | Preparation |
| Example | Name | Analytical Data | Methods |
| 16 | 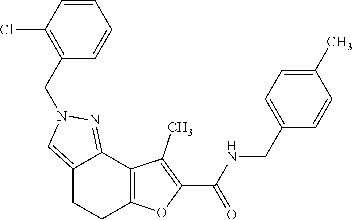 [SMILES: Cc1ccc(CNC(=O)c2oc3c(c2C)-c2nn(Cc4ccccc4Cl)cc2CC3)cc1] | ¹H NMR (400 MHz, DMSO-d6) δ [ppm]: 2.26 (s, 3H), 2.46 (s, 3H), 2.83-2.92 (m, 4H), 4.33 (d, 2H), 5.30 (s, 2H), 7.10-7.12 (m, 2H), 7.17-7.21 (m, 3H), 7.30-7.32 (m, 1H), 7.34-7.40 (m, 2H), 7.63 (s, 1H), 8.62 (t, 1H). LC-MS (Method A); Rₜ = 1.43 min, m/z = 446/448 [M + H]⁺ (Cl isotope pattern). | Intermediate 5 and CAS-RN: [104-84-7]; (conditions with HATU) 34 mg (54% yield) |
| | 2-[(3-chlorophenyl)methyl]-8-methyl- | | |
| | N-[(4-methylphenyl)methyl]-4,5- | | |
| | dihydro-2H-furo[2,3-g]indazole-7- | | |
| | carboxamide | | |
| 17 | 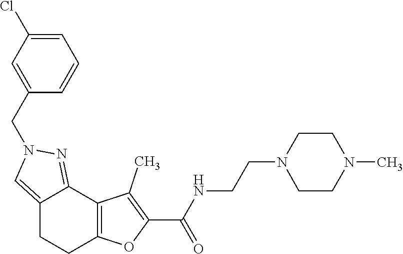 [SMILES: Cc1c(C(=O)NCCN2CCN(C)CC2)oc2c1-c1nn(Cc3cccc(Cl)c3)cc1CC2] | ¹H NMR (400 MHz, DMSO-d6) δ [ppm]: 2.13 (s, 3H), 2.19-2.45 (m, 13H), 2.84-2.92 (m, 4H), 3.27- 3.30 (m, 2H), 5.30 (s, 2H), 7.19-7.21 (m, 1H), 7.31 (m_{c}, 1H), 7.34-7.40 (m, 2H), 7.63 (s, 1H), 7.92 (t, 1H). LC-MS (Method A); Rₜ = 1.14 min, m/z = 468/470 [M + H]⁺ (Cl isotope pattern). | Intermediate 5 and CAS-RN: [934-98-5]; (conditions with HATU) 31 mg (47% yield) |
| | 2-[(3-chlorophenyl)methyl]-8-methyl- | | |
| | N-[2-(4-methylpiperazin-1-yl)ethyl]- | | |
| | 4,5-dihydro-2H-furo[2,3-g]indazole- | | |
| | 7-carboxamide | | |
| 18 | 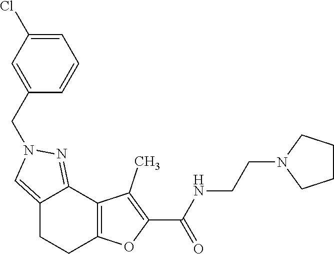 [SMILES: Cc1c(C(=O)NCCN2CCCC2)oc2c1-c1nn(Cc3cccc(Cl)c3)cc1CC2] | ¹H NMR (400 MHz, DMSO-d6) δ [ppm]: 1.65-1.68 (m, 4H), 2.43- 2.46 (m, 7H), 2.87-2.89 (m, 4H), 3.28-3.31 (m, 4H), 5.30 (s, 2H), 7.19-7.21 (m, 1H), 7.31-7.32 (m, 1H), 7.34-7.40 (m, 2H), 7.63 (s, 1H), 7.95 (t, 1H). LC-MS (Method A); Rₜ = 1.30 min, m/z = 439/441 [M + H]⁺ (Cl isotope pattern). | Intermediate 5 and CAS-RN: [7154-73-6]; (conditions with HATU) 28 mg (44% yield) |
| | 2-[(3-chlorophenyl)methyl]-8-methyl- | | |
| | N-[2-(pyrrolidin-1-yl)ethyl]-4,5- | | |
| | dihydro-2H-furo[2,3-g]indazole-7- | | |
| | carboxamide | | |
| 19 | 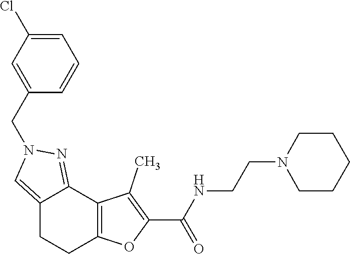 [SMILES: Cc1c(C(=O)NCCN2CCCCC2)oc2c1-c1nn(Cc3cccc(Cl)c3)cc1CC2] | ¹H NMR (400 MHz, DMSO-d6) δ [ppm]: 1.34-1.52 (m, 6H), 2.32- 2.39 (m, 6H), 2.45 (s, 3H), 2.83- 2.92 (m, 4H), 3.26-3.31 (m, 2H), 5.30 (s, 2H), 7.19-7.21 (m, 1H), 7.31-7.32 (m, 1H), 7.34-7.41 (m, 2H), 7.63 (s, 1H), 7.90 (t, 1H). LC-MS (Method A); Rₜ = 1.38 min, m/z = 453/455 [M + H]⁺ (Cl isotope pattern). | Intermediate 5 and CAS-RN: [27578-60-5]; (conditions with HATU) 33 mg (52% yield) |
| | 2-[(3-chlorophenyl)methyl]-8-methyl- | | |
| | N-[2-(piperidin-1-yl)ethyl]-4,5- | | |
| | dihydro-2H-furo[2,3-g]indazole-7- | | |
| | carboxamide | | |
| 20 | 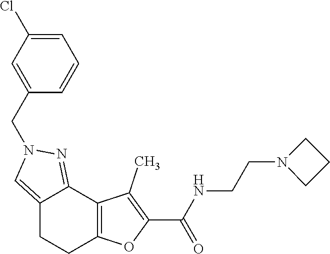 [SMILES: Cc1c(C(=O)NCCN2CCC2)oc2c1-c1nn(Cc3cccc(Cl)c3)cc1CC2] | ¹H NMR (400 MHz, DMSO-d6) δ [ppm]: 1.94 (quint, 2H), 2.42-2.45 (m, 5H), 2.85-2.90 (m, 4H), 3.08-3.14 (m, 6H), 5.30 (s, 2H), 7.19-7.21 (m, 1H), 7.31-7.32 (m, 1H), 7.33-7.40 (m, 2H), 7.63 (s, 1H), 7.87 (t, 1H). LC-MS (Method A); Rₜ = 1.23 min, m/z = 425/427 [M + H]⁺ (Cl isotope pattern). | Intermediate 5 and CAS-RN: [795299-77- 3]; (conditions with HATU) 25 mg (41% yield) |
| | N-[2-(azetidin-1-yl)ethyl]-2-[(3- | | |
| | chlorophenyl)methyl]-8-methyl-4,5- | | |
| | dihydro-2H-furo[2,3-g]indazole-7- | | |
| | carboxamide | | |

### Example 21

### 2-[(4-chlorophenyl)methyl]-8-methyl-N-[(2RS)-tetrahydrofuran-2-ylmethyl]-4,5-dihydro-2H-furo[2,3-g]indazole-7-carboxamide

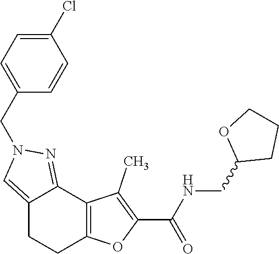 [SMILES: Cc1c(C(=O)NCC2CCCO2)oc2c1-c1nn(Cc3ccc(Cl)cc3)cc1CC2]

A solution of 2-[(4-chlorophenyl)methyl]-8-methyl-4,5-dihydro-2H-furo[2,3-g]indazole-7-carboxylic acid (Intermediate 6; 1.00 eq., 100 mg, 292 μmol) in DMF (1 mL) was treated with 1-[(2RS)-tetrahydrofuran-2-yl]methanamine (CAS No. [4795-29-3]; 1.2 eq., 36 μL, 350 μmol), HATU (CAS No. [148893-10-1]; 1.50 eq., 166 mg, 438 μmol) and N,N-diisopropylethylamine (CAS No. [7087-68-5]; 3.0 eq., 150 μL, 880 μmol) and stirred at rt overnight. The reaction mixture was diluted with EtOAc and the layers separated. The organic layer was washed with water, dried with Na₂SO₂, filtrated and concentrated under reduced pressure. The obtained residue was subjected to column chromatography (SiO₂, hexane/ethyl acetate) to give the title compound (48 mg, 35%).

¹H NMR (400 MHz, DMSO-d6) δ [ppm]: 1.55-1.61 (m, 1H), 1.76-1.91 (m, 3H), 2.45 (s, 3H), 2.83-2.91 (m, 4H), 3.19-3.26 (m, 2H), 3.58-3.63 (m, 1H), 3.73-3.78 (m, 1H), 3.91-3.97 (m, 1H), 5.28 (s, 2H), 7.24-7.28 (m, 2H), 7.40-7.43 (m, 2H), 7.60 (s, 1H), 7.98 (t, 1H).

UPLC-MS (Method 1): Rₜ=1.28 min; MS (ESIpos): m/z=426/428 [M+H]⁺ (CI isotope pattern).

The enantiomers of the racemic material of Example 21 were separated by chiral preparative HPLC (Instrument Labomatic HD5000, Labocord-5000; Gilson GX-241, Labcol Vario 4000; Column: Chiralpak IE 5 μm 250×30 mm; Eluent A: MTBE+0.1 vol % diethylamine; Eluent B: ethanol; Isocratic: 90% A+10% B; Flow: 40 mL/min; Detection: UV 254 nm) and analytically characterized by chiral HPLC (Instrument: Agilent 1260; Column: Chiralpak IE 3 μm 100×4.6 mm; Eluent A: MTBE+0.1 vol % diethylamine; Eluent B: ethanol; Isocratic: 90% A+10% B; Flow: 1.4 mL/min; Temperature: 25° C.; Detection: UV: 254 nm):

### Example 21-1

13 mg; Rₜ=2.48 min.

### Example 21-2

16 mg; Rt=2.67 min.

TABLE 5
The following examples (22 to 36) were prepared in analogy to Example
21 starting from Intermediate 6 and commercially available amines (or their salts).

| | Structure | | |
|---|---|---|---|
| | IUPAC- | | Preparation |
| Example | Name | Analytical Data | Methods |
| 22 | 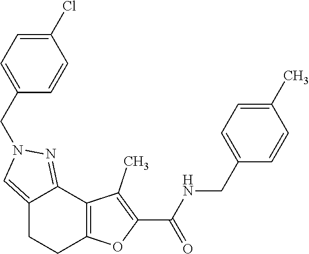 [SMILES: Cc1ccc(CNC(=O)c2oc3c(c2C)-c2nn(Cc4ccc(Cl)cc4)cc2CC3)cc1] | ¹H NMR (400 MHz, DMSO-d6) δ [ppm]: 2.26 (s, 3H), 2.46 (s, 3H), 2.83-2.91 (m, 4H), 4.33 (d, 2H), 5.28 (s, 2H), 7.10-7.12 (m, 2H), 7.17-7.19 (m, 2H), 7.24-7.27 (m, 2H), 7.40-7.43 (m, 2H), 7.60 (s, 1H), 8.62 (t, 1H). LC-MS (Method A); Rₜ = 1.43 min, m/z = 446/448 [M + H]⁺ (Cl isotope pattern). | Intermediate 6 and CAS-RN: [104-84-7]; (conditions with HATU) 25 mg (34% yield) |
| | 2-[(4-chlorophenyl)methyl]-8-methyl- | | |
| | N-[(4-methylphenyl)methyl]-4,5- | | |
| | dihydro-2H-furo[2,3-g]indazole-7- | | |
| | carboxamide | | |
| 23 | 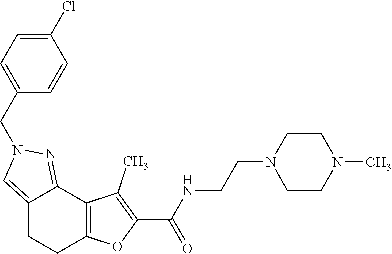 [SMILES: Cc1c(C(=O)NCCN2CCN(C)CC2)oc2c1-c1nn(Cc3ccc(Cl)cc3)cc1CC2] | ¹H NMR (400 MHz, DMSO-d6) δ [ppm]: 2.13 (s, 3H), 2.29-2.45 (m, 13H), 2.82-2.91 (m, 4H), 3.27- 3.32 (m, 2H), 5.28 (s, 2H), 7.25- 7.27 (m, 2H), 7.40-7.43 (m, 2H), 7.60 (s, 1H), 7.92 (t, 1H). LC-MS (Method A); Rₜ = 1.14 min, m/z = 468/470 [M + H]⁺ (Cl isotope pattern). | Intermediate 6 and CAS-RN: [934-98-5]; (conditions with HATU) 23 mg (31% yield) |
| | 2-[(4-chlorophenyl)methyl]-8-methyl- | | |
| | N-[2-(4-methylpiperazin-1-yl)ethyl]- | | |
| | 4,5-dihydro-2H-furo[2,3-g]indazole- | | |
| | 7-carboxamide | | |
| 24 | 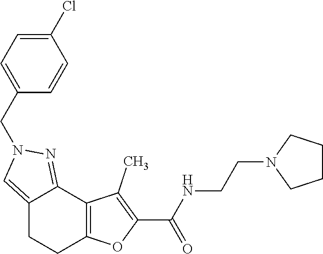 [SMILES: Cc1c(C(=O)NCCN2CCCC2)oc2c1-c1nn(Cc3ccc(Cl)cc3)cc1CC2] | ¹H NMR (400 MHz, DMSO-d6) δ [ppm]: 1.62-1.71 (m, 4H), 2.45 (mc, 7H), 2.82-2.91 (m, 4H), 3.27- 3.31 (m, 4H), 5.28 (s, 2H), 7.25- 7.27 (m, 2H), 7.40-7.43 (m, 2H), 7.60 (s, 1H), 7.95 (t, 1H). LC-MS (Method A); Rₜ = 1.31 min, m/z = 439/441 [M + H]⁺ (Cl isotope pattern). | Intermediate 6 and CAS-RN: [7154-73-6]; (conditions with HATU) 20 mg (29% yield) |
| | 2-[(4-chlorophenyl)methyl]-8-methyl- | | |
| | N-[2-(pyrrolidin-1-yl)ethyl]-4,5- | | |
| | dihydro-2H-furo[2,3-g]indazole-7- | | |
| | carboxamide | | |
| 25 | 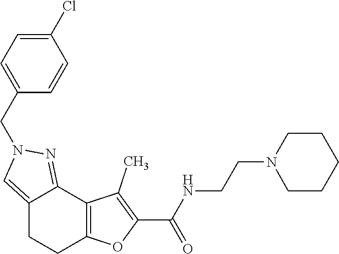 [SMILES: Cc1c(C(=O)NCCN2CCCCC2)oc2c1-c1nn(Cc3ccc(Cl)cc3)cc1CC2] | ¹H NMR (400 MHz, DMSO-d6) δ [ppm]: 1.35-1.39 (m, 2H), 1.45- 1.51 (m, 4H), 2.32-2.39 (m, 6H), 2.45 (s, 3H), 2.82-2.91 (m, 4H), 3.26-3.31 (m, 2H), 5.28 (s, 2H), 7.24-7.28 (m, 2H), 7.40-7.43 (m, 2H), 7.60 (s, 1H), 7.90 (t, 1H). LC-MS (Method A); Rₜ = 1.39 min, m/z = 453/455 [M + H]⁺ (Cl isotope pattern). | Intermediate 6 and CAS-RN: [27578-60-5]; (conditions with HATU) 25 mg (35% yield) |
| | 2-[(4-chlorophenyl)methyl]-8-methyl- | | |
| | N-[2-(piperidin-1-yl)ethyl]-4,5- | | |
| | dihydro-2H-furo[2,3-g]indazole-7- | | |
| | carboxamide | | |
| 26 | 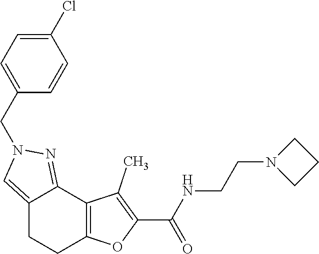 [SMILES: Cc1c(C(=O)NCCN2CCC2)oc2c1-c1nn(Cc3ccc(Cl)cc3)cc1CC2] | ¹H NMR (400 MHz, DMSO-d6) δ [ppm]: 1.94 (quint, 2H), 2.42-2.45 (m, 5H), 2.84-2.90 (m, 4H), 3.08- 3.13 (m, 6H), 5.28 (s, 2H), 7.24- 7.27 (m, 2H), 7.40-7.43 (m, 2H), 7.60 (s, 1H), 7.87 (t, 1H). LC-MS (Method A); Rₜ = 1.24 min, m/z = 425/427 [M + H]⁺ (Cl isotope pattern). | Intermediate 6 and CAS-RN: [795299-77- 3]; (conditions with HATU) 12 mg (16% yield) |
| | N-[2-(azetidin-1-yl)ethyl]-2-[(4- | | |
| | chlorophenyl)methyl]-8-methyl-4,5- | | |
| | dihydro-2H-furo[2,3-g]indazole-7- | | |
| | carboxamide | | |
| 27 | 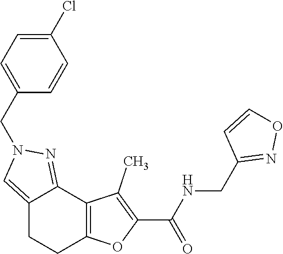 [SMILES: Cc1c(C(=O)NCc2ccon2)oc2c1-c1nn(Cc3ccc(Cl)cc3)cc1CC2] | ¹H NMR (400 MHz, DMSO-d6) δ [ppm]: 2.47 (s, 3H), 2.83-2.92 (m, 4H), 4.46 (d, 2H), 5.29 (s, 2H), 6.49 (d, 1H), 7.24-7.28 (m, 2H), 7.40-7.43 (m, 2H), 7.61 (s, 1H), 8.71 (t, 1H), 8.82 (d, 1H). LC-MS (Method A); Rₜ = 1.20 min, m/z = 423/425 [M + H]⁺ (Cl isotope pattern). | Intermediate 6 and CAS-RN: [1187933-48- 7]; (conditions with HATU) 24 mg (36% yield) |
| | 2-[(4-chlorophenyl)methyl]-8-methyl- | | |
| | N-[(1,2-oxazol-3-yl)methyl]-4,5- | | |
| | dihydro-2H-furo[2,3-g]indazole-7- | | |
| | carboxamide | | |
| 28 | 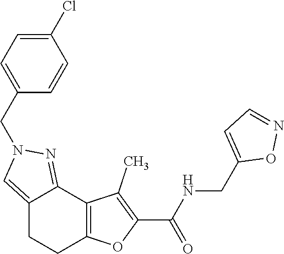 [SMILES: Cc1c(C(=O)NCc2ccno2)oc2c1-c1nn(Cc3ccc(Cl)cc3)cc1CC2] | ¹H NMR (400 MHz, DMSO-d6) δ [ppm]: 2.46 (s, 3H), 2.84-2.92 (m, 4H), 4.53 (d, 2H), 5.29 (s, 2H), 6.32 (m_{c}, 1H), 7.24-7.28 (m, 2H), 7.40-7.43 (m, 2H), 7.61 (s, 1H), 8.47 (d, 1H), 8.79 (t, 1H). LC-MS (Method A); Rₜ = 1.19 min, m/z = 423/425 [M + H]⁺ (Cl isotope pattern). | Intermediate 6 and CAS-RN: [440099-32- 1]; (conditions with HATU) 17 mg (24% yield) |
| | 2-[(4-chlorophenyl)methyl]-8-methyl- | | |
| | N-[(1,2-oxazol-5-yl)methyl]-4,5- | | |
| | dihydro-2H-furo[2,3-g]indazole-7- | | |
| | carboxamide | | |
| 29 | 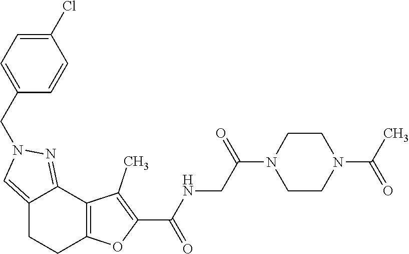 [SMILES: CC(=O)N1CCN(C(=O)CNC(=O)c2oc3c(c2C)-c2nn(Cc4ccc(Cl)cc4)cc2CC3)CC1] | ¹H NMR (400 MHz, DMSO-d6) δ [ppm]: 2.02-2.03 (m, 3H), 2.46 (s, 3H), 2.84-2.93 (m, 4H), 3.43-3.49 (m, 8H), 4.08-4.10 (m, 2H), 5.29 (s, 2H), 7.23-7.28 (m, 2H), 7.40- 7.43 (m, 2H), 7.61 (s, 1H), 7.93 (t, 1H). LC-MS (Method A); Rₜ = 1.07 min, m/z = 510/512 [M + H]⁺ (Cl isotope pattern). | Intermediate 6 and CAS-RN: [896508-00- 2]; (conditions with HATU) 37 mg (32% yield) |
| | N-[2-(4-acetylpiperazin-1-yl)-2- | | |
| | oxoethyl]-2-[(4-chlorophenyl)me- | | |
| | thyl]-8-methyl-4,5-dihydro-2H- | | |
| | furo[2,3-g]indazole-7-carboxamide | | |
| 30 | 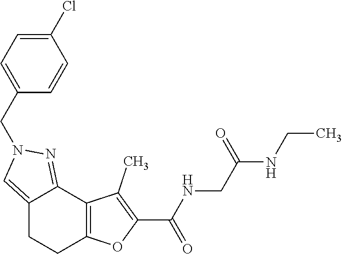 [SMILES: CCNC(=O)CNC(=O)c1oc2c(c1C)-c1nn(Cc3ccc(Cl)cc3)cc1CC2] | ¹H NMR (400 MHz, DMSO-d6) δ [ppm]: 1.01 (t, 3H), 2.46 (s, 3H), 2.84-2.92 (m, 4H), 3.05-3.12 (m, 2H), 3.76 (d, 2H), 5.29 (s, 2H), 7.25-7.28 (m, 2H), 7.40-7.43 (m, 2H), 7.61 (s, 1H), 7.88 (t, 1H), 8.11 (t, 1H). LC-MS (Method A); Rₜ = 1.10 min, m/z = 427/429 [M + H]⁺ (Cl isotope pattern). | Intermediate 6 and CAS-RN: [26595-78-8]; (conditions with HATU) 59 mg (61% yield) |
| | 2-[(4-chlorophenyl)methyl]-N-[2- | | |
| | (ethylamino)-2-oxoethyl]-8-methyl- | | |
| | 4,5-dihydro-2H-furo[2,3-g]indazole- | | |
| | 7-carboxamide | | |
| 31 | 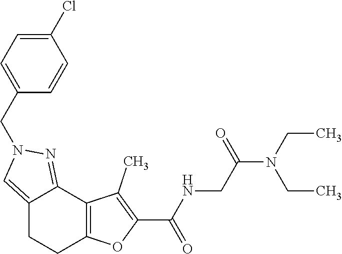 [SMILES: CCN(CC)C(=O)CNC(=O)c1oc2c(c1C)-c1nn(Cc3ccc(Cl)cc3)cc1CC2] | ¹H NMR (400 MHz, DMSO-d6) δ [ppm]: 1.03 (t, 3H), 1.14 (t, 3H), 2.46 (s, 3H), 2.84-2.93 (m, 4H), 3.26-3.34 (m, 4H), 4.04 (d, 2H), 5.29 (s, 2H), 7.24-7.28 (m, 2H), 7.40-7.43 (m, 2H), 7.61 (s, 1H), 7.86 (t, 1H). LC-MS (Method A); Rₜ = 1.25 min, m/z = 455/457 [M + H]⁺ (Cl isotope pattern). | Intermediate 6 and CAS-RN: [34105-57-2]; (conditions with HATU) 39 mg (38% yield) |
| | 2-[(4-chlorophenyl)methyl]-N-[2- | | |
| | (diethylamino)-2-oxoethyl]-8-methyl- | | |
| | 4,5-dihydro-2H-furo[2,3-g]indazole- | | |
| | 7-carboxamide | | |
| 32 | 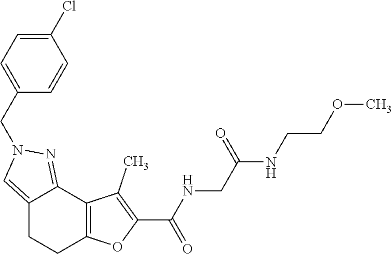 [SMILES: COCCNC(=O)CNC(=O)c1oc2c(c1C)-c1nn(Cc3ccc(Cl)cc3)cc1CC2] | ¹H NMR (400 MHz, DMSO-d6) δ [ppm]: 2.46 (s, 3H), 2.84-2.93 (m, 4H), 3.21-3.25 (m, 5H), 3.32-3.35 (m, 2H), 3.78 (d, 2H), 5.29 (s, 2H), 7.25-7.28 (m, 2H), 7.40-7.43 (m, 2H), 7.61 (s, 1H), 7.96 (t, 1H), 8.12 (t, 1H). LC-MS (Method A); Rₜ = 1.08 min, m/z = 457/459 [M + H]⁺ (Cl isotope pattern). | Intermediate 6 and CAS-RN: [1220037-70- 6]; (conditions with HATU) 56 mg (54% yield) |
| | 2-[(4-chlorophenyl)methyl]-N-{2-[(2- | | |
| | methoxyethyl)amino]-2-oxoethyl}-8- | | |
| | methyl-4,5-dihydro-2H-furo[2,3- | | |
| | g]indazole-7-carboxamide | | |
| 33 | 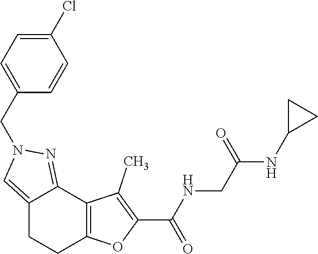 [SMILES: Cc1c(C(=O)NCC(=O)NC2CC2)oc2c1-c1nn(Cc3ccc(Cl)cc3)cc1CC2] | ¹H NMR (400 MHz, DMSO-d6) δ [ppm]: 0.39-0.43 (m, 2H), 0.58- 0.63 (m, 2H), 2.46 (s, 3H), 2.59- 2.65 (m, 1H), 2.84-2.92 (m, 4H), 3.73 (d, 2H), 5.29 (s, 2H), 7.25- 7.28 (m, 2H), 7.40-7.43 (m, 2H), 7.61 (s, 1H), 7.98 (d, 1H), 8.05 (t, 1H). UPLC-MS (Method 1); Rₜ = 1.15 min, m/z = 439/441 [M + H]⁺ (Cl isotope pattern). | Intermediate 6 and CAS-RN: [670253-51- 7]; (conditions with HATU) 62 mg (61% yield) |
| | 2-[(4-chlorophenyl)methyl]-N-[2- | | |
| | (cyclopropylamino)-2-oxoethyl]-8- | | |
| | methyl-4,5-dihydro-2H-furo[2,3- | | |
| | g]indazole-7-carboxamide | | |
| 34 | 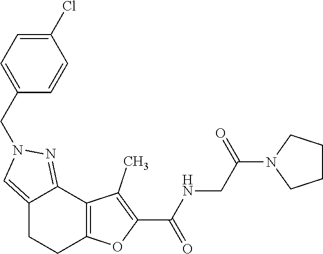 [SMILES: Cc1c(C(=O)NCC(=O)N2CCCC2)oc2c1-c1nn(Cc3ccc(Cl)cc3)cc1CC2] | ¹H NMR (400 MHz, DMSO-d6) δ [ppm]: 1.74-1.81 (m, 2H), 1.87- 1.93 (m, 2H), 2.46 (s, 3H), 2.84- 2.93 (m, 4H), 3.31 (t, 2H), 3.43 (t, 2H), 3.96 (d, 2H), 5.29 (s, 2H), 7.24-7.28 (m, 2H), 7.40-7.43 (m, 2H), 7.61 (s, 1H), 7.89 (t, 1H). UPLC-MS (Method 1); Rₜ = 1.21 min, m/z = 453/455 [M + H]⁺ (Cl isotope pattern). | Intermediate 6 and CAS-RN: [35855-14-2]; (conditions with HATU) 56 mg (54% yield) |
| | 2-[(4-chlorophenyl)methyl]-8-methyl- | | |
| | N-[2-oxo-2-(pyrrolidin-1-yl)ethyl]- | | |
| | 4,5-dihydro-2H-furo[2,3-g]indazole- | | |
| | 7-carboxamide | | |
| 35 | 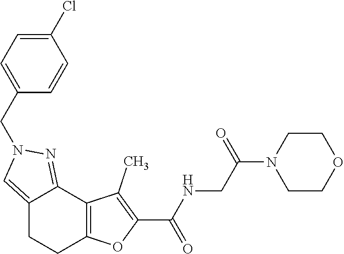 [SMILES: Cc1c(C(=O)NCC(=O)N2CCOCC2)oc2c1-c1nn(Cc3ccc(Cl)cc3)cc1CC2] | ¹H NMR (400 MHz, DMSO-d6) δ [ppm]: 2.46 (s, 3H), 2.84-2.93 (m, 4H), 3.44-3.47 (m, 4H), 3.55-3.61 (m, 4H), 4.06 (d, 2H), 5.29 (s, 2H), 7.24-7.28 (m, 2H), 7.40-7.43 (m, 2H), 7.61 (s, 1H), 7.92 (t, 1H). LC-MS (Method A); Rₜ = 1.16 min, m/z = 469/471 [M + H]⁺ (Cl isotope pattern). | Intermediate 6 and CAS-RN: [24152-96-3]; (conditions with HATU) 53 mg (49% yield) |
| | 2-[(4-chlorophenyl)methyl]-8-methyl- | | |
| | N-[2-(morpholin-4-yl)-2-oxoethyl]- | | |
| | 4,5-dihydro-2H-furo[2,3-g]indazole- | | |
| | 7-carboxamide | | |
| 36 | 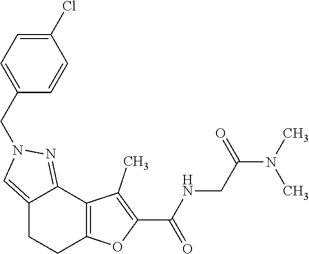 [SMILES: Cc1c(C(=O)NCC(=O)N(C)C)oc2c1-c1nn(Cc3ccc(Cl)cc3)cc1CC2] | ¹H NMR (400 MHz, DMSO-d6) δ [ppm]: 2.46 (s, 3H), 2.84-2.93 (m, 7H), 2.98 (s, 3H), 4.03 (d, 2H), 5.29 (s, 2H), 7.24-7.28 (m, 2H), 7.40-7.43 (m, 2H), 7.61 (s, 1H), 7.84 (t, 1H). LC-MS (Method A); Rₜ = 1.13 min, m/z = 427/429 [M + H]⁺ (Cl isotope pattern). | Intermediate 6 and CAS-RN: [1857-19-1]; (conditions with HATU) 21 mg (22% yield) |
| | 2-[(4-chlorophenyl)methyl]-N-[2- | | |
| | (dimethylamino)-2-oxoethyl]-8- | | |
| | methyl-4,5-dihydro-2H-furo[2,3- | | |
| | g]indazole-7-carboxamide | | |

### Example 37

### 2-[(4-chlorophenyl)methyl]-8-methyl-N-[(1-methyl-1H-pyrazol-3-yl)methyl]-4,5-dihydro-2H-furo[2,3-g]indazole-7-carboxamide

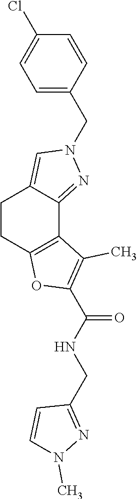 [SMILES: Cc1c(C(=O)NCc2ccn(C)n2)oc2c1-c1nn(Cc3ccc(Cl)cc3)cc1CC2]

To a solution of 2-[(4-chlorophenyl)methyl]-8-methyl-4,5-dihydro-2H-furo[2,3-g]indazole-7-carboxylic acid (51 mg, 150 μmol; Intermediate 6) in N,N-dimethylformamide a solution of HATU (114 mg, 300 μmol; CAS-RN:[148893-10-1]) in N,N-dimethylformamide followed by N,N-diisopropylethylamine (77.5 mg, 600 μmol; CAS-RN:[7087-68-5]) were added. The reaction mixture was stirred at rt for 10 minutes and subsequently added to 1-(1-methyl-1H-pyrazol-3-yl)methanamine (33.3 mg, 300 μmol) in a glass vial. The glass vial was put in a metal block and the block was placed on a shaker for 1 day at room temperature. The reaction mixture was filtered through a pad of Celite and purified by HPLC to afford 15.8 mg (24% yield, 98% purity) of the title compound.

UPLC-MS (Method 1): Rₜ=1.16 min; MS (ESIpos): m/z=436 [M+H]⁺.

TABLE 6
The following examples (38 to 91) were prepared in analogy
to Example 37 starting from Intermediate 6 and commercially
available amines (or their salts).

| | | | |
|---|---|---|---|
| 38 | 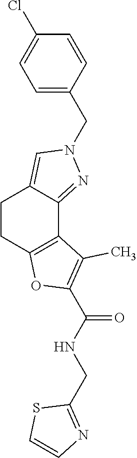 [SMILES: Cc1c(C(=O)NCc2nccs2)oc2c1-c1nn(Cc3ccc(Cl)cc3)cc1CC2] | UPLC-MS (Method 1): Rₜ = 1.20 min; MS (ESIpos): m/z = 439 [M + H]⁺. | Intermediate 6 and CAS- RN: [55661- 33-1]; (conditions with HATU) 13.8 mg (19% yield, 92% purity) |
| | 2-[(4-chlorophenyl)methyl]-8-methyl- | | |
| | N-[(1,3-thiazol-2-yl)methyl]-4,5- | | |
| | dihydro-2H-furo[2,3-g]indazole-7- | | |
| | carboxamide | | |
| 39 | 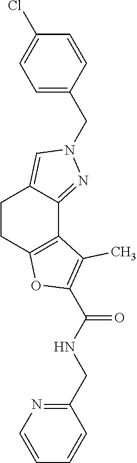 [SMILES: Cc1c(C(=O)NCc2ccccn2)oc2c1-c1nn(Cc3ccc(Cl)cc3)cc1CC2] | UPLC-MS (Method 1): Rₜ = 1.21 min; MS (ESIpos): m/z = 433 [M + H]⁺. | Intermediate 6 and CAS-RN: [3731-51-9]; (conditions with HATU) 12.5 mg (18% yield, 95% purity) |
| | 2-[(4-chlorophenyl)methyl]-8-methyl- | | |
| | N-[(pyridin-2-yl)methyl]-4,5-dihydro- | | |
| | 2H-furo[2,3-g]indazole-7- | | |
| | carboxamide | | |
| 40 | 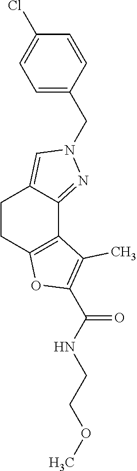 | UPLC-MS (Method 1): Rₜ = 1.19 min; MS (ESIpos): m/z = 400 [M + H]⁺. | Intermediate 6 and CAS-RN: [109-85-3]; (conditions with HATU) 10.1 mg (16% yield, 98% purity) |
| | 2-[(4-chlorophenyl)methyl]-N-(2- | | |
| | methoxyethyl)-8-methyl-4,5-dihydro- | | |
| | 2H-furo[2,3-g]indazole-7- | | |
| | carboxamide | | |
| 41 | 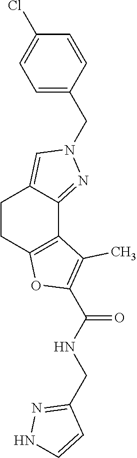 [SMILES: Cc1c(C(=O)NCc2cc[nH]n2)oc2c1-c1nn(Cc3ccc(Cl)cc3)cc1CC2] | UPLC-MS (Method 1): Rₜ = 1.10 min; MS (ESIpos): m/z = 422 [M + H]⁺. | Intermediate 6 and CAS-RN: [37599-58- 9]; (conditions with HATU) 16.2 mg (25% yield, 98% purity) |
| | 2-[(4-chlorophenyl)methyl]-8-methyl- | | |
| | N-[(1H-pyrazol-3-yl)methyl]-4,5- | | |
| | dihydro-2H-furo[2,3-g]indazole-7- | | |
| | carboxamide | | |
| 42 | 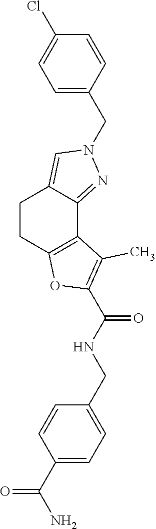 [SMILES: Cc1c(C(=O)NCc2ccc(C(N)=O)cc2)oc2c1-c1nn(Cc3ccc(Cl)cc3)cc1CC2] | UPLC-MS (Method 1): Rₜ = 1.11 min; MS (ESIpos): m/z = 475 [M + H]⁺. | Intermediate 6 and CAS-RN: [369-53-9]; (conditions with HATU) 15.3 mg (19% yield, 86% purity) |
| | N-[(4-carbamoylphenyl)methyl]-2- | | |
| | [(4-chlorophenyl)methyl]-8-methyl- | | |
| | 4,5-dihydro-2H-furo[2,3-g]indazole- | | |
| | 7-carboxamide | | |
| 43 | 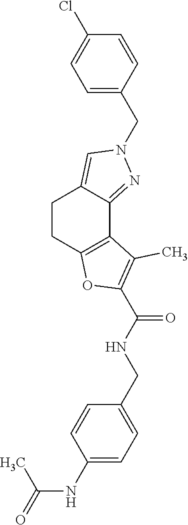 [SMILES: CC(=O)Nc1ccc(CNC(=O)c2oc3c(c2C)-c2nn(Cc4ccc(Cl)cc4)cc2CC3)cc1] | UPLC-MS (Method 1): Rₜ = 1.17 min; MS (ESIpos): m/z = 489 [M + H]⁺. | Intermediate 6 and CAS-RN: [25027- 73-0]; (conditions with HATU) 3.9 mg (5% yield, 91% purity) |
| | N-[(4-acetamidophenyl)methyl]-2- | | |
| | [(4-chlorophenyl)methyl]-8-methyl- | | |
| | 4,5-dihydro-2H-furo[2,3-g]indazole- | | |
| | 7-carboxamide | | |
| 44 | 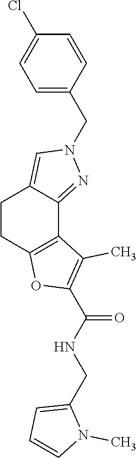 [SMILES: Cc1c(C(=O)NCc2cccn2C)oc2c1-c1nn(Cc3ccc(Cl)cc3)cc1CC2] | UPLC-MS (Method 1): Rₜ = 1.31 min; MS (ESIpos): m/z = 435 [M + H]⁺. | Intermediate 6 and CAS-RN: [69807- 81-4]; (conditions with HATU) 5.0 mg (7% yield, 96% purity) |
| | 2-[(4-chlorophenyl)methyl]-8-methyl- | | |
| | N-[(1-methyl-1H-pyrrol-2-yl)methyl]- | | |
| | 4,5-dihydro-2H-furo[2,3-g]indazole- | | |
| | 7-carboxamide | | |
| 45 | 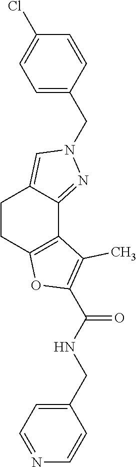 [SMILES: Cc1c(C(=O)NCc2ccncc2)oc2c1-c1nn(Cc3ccc(Cl)cc3)cc1CC2] | UPLC-MS (Method 1): Rₜ = 1.15 min; MS (ESIpos): m/z = 433 [M + H]⁺. | Intermediate 6 and CAS-RN: [3731-53-1]; (conditions with HATU) 8.7 mg (13% yield, 96% purity) |
| | 2-[(4-chlorophenyl)methyl]-8-methyl- | | |
| | N-[(pyridin-4-yl)methyl]-4,5-dihydro- | | |
| | 2H-furo[2,3-g]indazole-7- | | |
| | carboxamide | | |
| 46 | 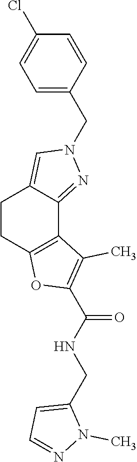 [SMILES: Cc1c(C(=O)NCc2ccnn2C)oc2c1-c1nn(Cc3ccc(Cl)cc3)cc1CC2] | UPLC-MS (Method 1): Rₜ = 1.15 min; MS (ESIpos): m/z = 436 [M + H]⁺. | Intermediate 6 and CAS-RN: [863548-52- 1]; (conditions with HATU) 15.9 mg (24% yield, 98% purity) |
| | 2-[(4-chlorophenyl)methyl]-8-methyl- | | |
| | N-[(1-methyl-1H-pyrazol-5- | | |
| | yl)methyl]-4,5-dihydro-2H-furo[2,3- | | |
| | g]indazole-7-carboxamide | | |
| 47 | 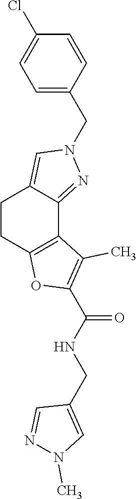 [SMILES: Cc1c(C(=O)NCc2cnn(C)c2)oc2c1-c1nn(Cc3ccc(Cl)cc3)cc1CC2] | UPLC-MS (Method 1): Rₜ = 1.13 min; MS (ESIpos): m/z = 436 [M + H]⁺. | Intermediate 6 and CAS-RN: [400877-05- 6]; (conditions with HATU) 20.6 mg (31% yield, 99% purity) |
| | 2-[(4-chlorophenyl) | | |
| | methyl]-8-methyl- | | |
| | N-[(1-methyl-1H-pyrazol-4- | | |
| | yl)methyl]-4,5-dihydro-2H-furo | | |
| | [2,3-g]indazole-7-carboxamide | | |
| 48 | 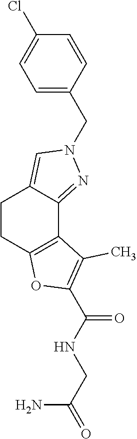 [SMILES: Cc1c(C(=O)NCC(N)=O)oc2c1-c1nn(Cc3ccc(Cl)cc3)cc1CC2] | UPLC-MS (Method 1): Rₜ = 1.01 min; MS (ESIpos): m/z = 399 [M + H]⁺. | Intermediate 6 and CAS-RN: [1668-10-6]; (conditions with HATU) 13.5 mg (22% yield, 97% purity) |
| | N-(2-amino-2-oxoethyl)-2-[(4- | | |
| | chlorophenyl)methyl]-8-methyl-4,5- | | |
| | dihydro-2H-furo[2,3-g]indazole-7- | | |
| | carboxamide | | |
| 49 | 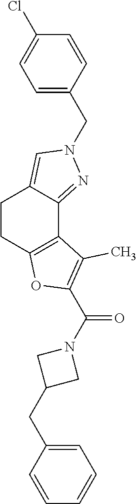 [SMILES: Cc1c(C(=O)N2CC(Cc3ccccc3)C2)oc2c1-c1nn(Cc3ccc(Cl)cc3)cc1CC2] | UPLC-MS (Method 1): Rₜ = 1.47 min; MS (ESIpos): m/z = 472 [M + H]⁺. | Intermediate 6 and CAS-RN: [90874- 34-3]; (conditions with HATU) 23.0 mg (30% yield, 94% purity) |
| | (3-benzylazetidin-1-yl){2-[(4- | | |
| | chlorophenyl)methyl]-8-methyl-4,5- | | |
| | dihydro-2H-furo[2,3-g]indazol-7- | | |
| | yl}methanone | | |
| 50 | 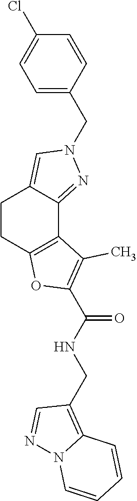 [SMILES: Cc1c(C(=O)NCc2cnn3ccccc23)oc2c1-c1nn(Cc3ccc(Cl)cc3)cc1CC2] | UPLC-MS (Method 1): Rₜ = 1.23 min; MS (ESIpos): m/z = 472 [M + H]⁺. | Intermediate 6 and CAS-RN: [1351659- 25-0]; (conditions with HATU) 14.2 mg (19% yield, 93% purity) |
| | 2-[(4-chlorophenyl) | | |
| | methyl]-8-methyl- | | |
| | N-[(pyrazolo[1,5-a]pyridin-3-yl) | | |
| | methyl]-4,5-dihydro-2H-furo[2,3- | | |
| | g]indazole-7-carboxamide | | |
| 51 | 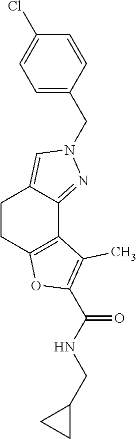 [SMILES: Cc1c(C(=O)NCC2CC2)oc2c1-c1nn(Cc3ccc(Cl)cc3)cc1CC2] | UPLC-MS (Method 1): Rₜ = 1.31 min; MS (ESIpos): m/z = 396 [M + H]⁺. | Intermediate 6 and CAS-RN: [2516-47-4]; (conditions with HATU) 16.6 mg (27% yield, 95% purity) |
| | 2-[(4-chlorophenyl)methyl]-N- | | |
| | (cyclopropylmethyl)-8-methyl-4,5- | | |
| | dihydro-2H-furo[2,3-g]indazole-7- | | |
| | carboxamide | | |
| 52 | 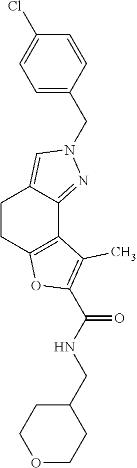 [SMILES: Cc1c(C(=O)NCC2CCOCC2)oc2c1-c1nn(Cc3ccc(Cl)cc3)cc1CC2] | UPLC-MS (Method 1): Rₜ = 1.22 min; MS (ESIpos): m/z = 440 [M + H]⁺. | Intermediate 6 and CAS-RN: [130290-79- 8]; (conditions with HATU) 12.0 mg (18% yield, 97% purity) |
| | 2-[(4-chlorophenyl)methyl]-8-methyl- | | |
| | N-[(oxan-4-yl)methyl]-4,5-dihydro- | | |
| | 2H-furo[2,3-g]indazole-7- | | |
| | carboxamide | | |
| 53 | 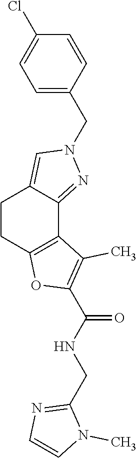 [SMILES: Cc1c(C(=O)NCc2nccn2C)oc2c1-c1nn(Cc3ccc(Cl)cc3)cc1CC2] | UPLC-MS (Method 1): Rₜ = 1.11 min; MS (ESIpos): m/z = 436 [M + H]⁺. | Intermediate 6 and CAS-RN: [124312-73- 8]; (conditions with HATU) 13.2 mg (20% yield, 98% purity) |
| | 2-[(4-chlorophenyl)methyl]-8-methyl- | | |
| | N-[(1-methyl-1H-imidazol-2- | | |
| | yl)methyl]-4,5-dihydro-2H-furo[2,3- | | |
| | g]indazole-7-carboxamide | | |
| 54 | 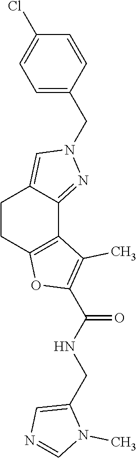 [SMILES: Cc1c(C(=O)NCc2cncn2C)oc2c1-c1nn(Cc3ccc(Cl)cc3)cc1CC2] | UPLC-MS (Method 1): Rₜ = 1.08 min; MS (ESIpos): m/z = 436 [M + H]⁺. | Intermediate 6 and CAS-RN: [486414-86- 2]; (conditions with HATU) 14.9 mg (22% yield, 98% purity) |
| | 2-[(4-chlorophenyl)methyl]-8-methyl- | | |
| | N-[(1-methyl-1H-imidazol-5- | | |
| | yl)methyl]-4,5-dihydro-2H-furo[2,3- | | |
| | g]indazole-7-carboxamide | | |
| 55 | 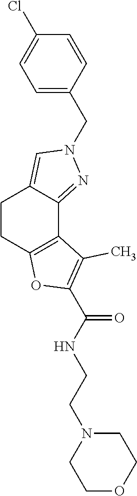 [SMILES: Cc1c(C(=O)NCCN2CCOCC2)oc2c1-c1nn(Cc3ccc(Cl)cc3)cc1CC2] | UPLC-MS (Method 1): Rₜ = 1.15 min; MS (ESIpos): m/z = 455 [M + H]⁺. | Intermediate 6 and CAS-RN: [2038-03-1]; (conditions with HATU) 18.8 mg (28% yield, 100% purity) |
| | 2-[(4-chlorophenyl)methyl]-8-methyl- | | |
| | N-[2-(morpholin-4-yl)ethyl]-4,5- | | |
| | dihydro-2H-furo[2,3-g]indazole-7- | | |
| | carboxamide | | |
| 56 | 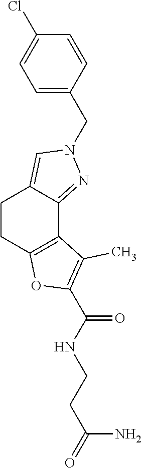 [SMILES: Cc1c(C(=O)NCCC(N)=O)oc2c1-c1nn(Cc3ccc(Cl)cc3)cc1CC2] | UPLC-MS (Method 1): Rₜ = 1.01 min; MS (ESIpos): m/z = 413 [M + H]⁺. | Intermediate 6 and CAS-RN: [4726-85-6]; (conditions with HATU) 10.7 mg (16% yield, 94% purity) |
| | N-(3-amino-3-oxopropyl)-2-[(4- | | |
| | chlorophenyl)methyl]-8-methyl-4,5- | | |
| | dihydro-2H-furo[2,3-g]indazole-7- | | |
| | carboxamide | | |
| 57 | 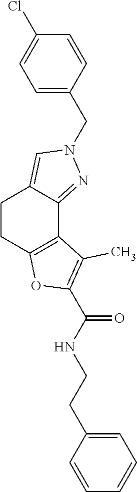 [SMILES: Cc1c(C(=O)NCCc2ccccc2)oc2c1-c1nn(Cc3ccc(Cl)cc3)cc1CC2] | UPLC-MS (Method 1): Rₜ = 1.39 min; MS (ESIpos): m/z = 446 [M + H]⁺. | Intermediate 6 and CAS-RN: [64-04-0]; (conditions with HATU) 7.5 mg (10% yield, 93% purity) |
| | 2-[(4-chlorophenyl)methyl]-8-methyl- | | |
| | N-(2-phenylethyl)-4,5-dihydro-2H- | | |
| | furo[2,3-g]indazole-7-carboxamide | | |
| 58 | 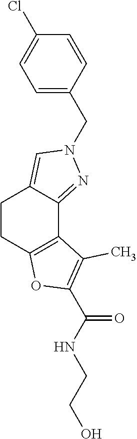 [SMILES: Cc1c(C(=O)NCCO)oc2c1-c1nn(Cc3ccc(Cl)cc3)cc1CC2] | UPLC-MS (Method 1): Rₜ = 1.05 min; MS (ESIpos): m/z = 386 [M + H]⁺. | Intermediate 6 and CAS-RN: [141-43-5]; (conditions with HATU) 12.9 mg (22% yield, 99% purity) |
| | 2-[(4-chlorophenyl)methyl]-N-(2- | | |
| | hydroxyethyl)-8-methyl-4,5-dihydro- | | |
| | 2H-furo[2,3-g]indazole-7- | | |
| | carboxamide | | |
| 59 | 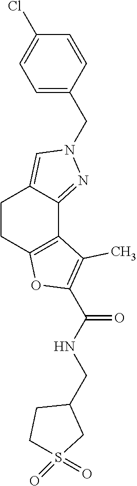 [SMILES: Cc1c(C(=O)NCC2CCS(=O)(=O)C2)oc2c1-c1nn(Cc3ccc(Cl)cc3)cc1CC2] | UPLC-MS (Method 1): Rₜ = 1.11 min; MS (ESIpos): m/z = 474 [M + H]⁺. | Intermediate 6 and CAS-RN: [45697- 13-0]; (conditions with HATU) 13.4 mg (18% yield, 94% purity) |
| | 2-(4-chlorobenzyl)-N-[(1,1- | | |
| | dioxidotetrahydrothiophen-3- | | |
| | yl)methyl]-8-methyl-4,5-dihydro-2H- | | |
| | furo[2,3-g]indazole-7-carboxamide | | |
| 60 | 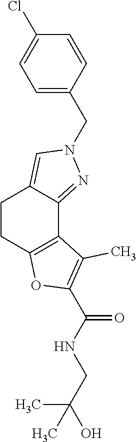 [SMILES: Cc1c(C(=O)NCC(C)(C)O)oc2c1-c1nn(Cc3ccc(Cl)cc3)cc1CC2] | UPLC-MS (Method 1): Rₜ = 1.14 min; MS (ESIpos): m/z = 414 [M + H]⁺. | Intermediate 6 and CAS-RN: [2854- 16-2]; (conditions with HATU) 17.2 mg (27% yield, 98% purity) |
| | 2-[(4-chlorophenyl)methyl]-N-(2- | | |
| | hydroxy-2-methylpropyl)-8-methyl- | | |
| | 4,5-dihydro-2H-furo[2,3-g]indazole- | | |
| | 7-carboxamide | | |
| 61 | 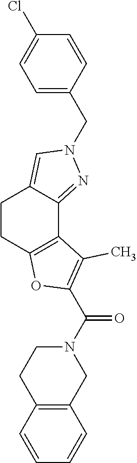 [SMILES: Cc1c(C(=O)N2CCc3ccccc3C2)oc2c1-c1nn(Cc3ccc(Cl)cc3)cc1CC2] | UPLC-MS (Method 1): Rₜ = 1.45 min; MS (ESIpos): m/z = 458 [M + H]⁺. | Intermediate 6 and CAS-RN: [91-21-4]; (conditions with HATU) 14.4 mg (20% yield, 96% purity) |
| | {2-[(4-chlorophenyl)methyl]-8- | | |
| | methyl-4,5-dihydro-2H-furo[2,3- | | |
| | g]indazol-7-yl}(3,4- | | |
| | dihydroisoquinolin-2(1H)- | | |
| | yl)methanone | | |
| 62 | 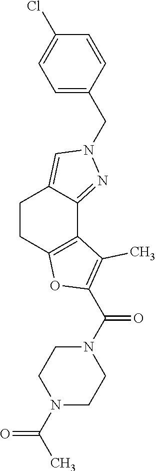 [SMILES: CC(=O)N1CCN(C(=O)c2oc3c(c2C)-c2nn(Cc4ccc(Cl)cc4)cc2CC3)CC1] | UPLC-MS (Method 1): Rₜ = 1.10 min; MS (ESIpos): m/z = 453 [M + H]⁺. | Intermediate 6 and CAS-RN: [13889- 98-0]; (conditions with HATU) 15.4 mg (23% yield, 100% purity) |
| | 1-(4-{2-[(4-chlorophenyl)methyl]-8- | | |
| | methyl-4,5-dihydro-2H-furo[2,3- | | |
| | g]indazole-7-carbonyl}piperazin-1- | | |
| | yl)ethan-1-one | | |
| 63 | 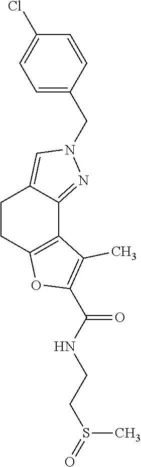 [SMILES: Cc1c(C(=O)NCCS(C)=O)oc2c1-c1nn(Cc3ccc(Cl)cc3)cc1CC2] | UPLC-MS (Method 1): Rₜ = 1.02 min; MS (ESIpos): m/z = 432 [M + H]⁺. | Intermediate 6 and CAS- RN: [49773- 19-5]; (conditions with HATU) 14.5 mg (22% yield, 98% purity) |
| | 2-[(4-chlorophenyl)methyl]-NV-[2- | | |
| | (methanesulfinyl)ethyl]-8-methyl- | | |
| | 4,5-dihydro-2H-furo[2,3-g]indazole- | | |
| | 7-carboxamide | | |
| 64 | 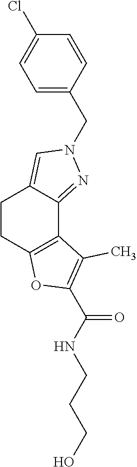 | UPLC-MS (Method 1): Rₜ = 1.08 min; MS (ESIpos): m/z = 400 [M + H]⁺. | Intermediate 6 and CAS-RN: [156-87-6]; (conditions with HATU) 15.5 mg (26% yield, 100% purity) |
| | 2-[(4-chlorophenyl)methyl]-N-(3- | | |
| | hydroxypropyl)-8-methyl-4,5- | | |
| | dihydro-2H-furo[2,3-g]indazole-7- | | |
| | carboxamide | | |
| 65 | 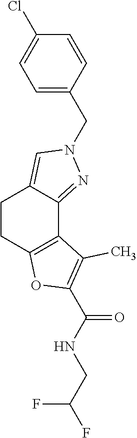 [SMILES: Cc1c(C(=O)NCC(F)F)oc2c1-c1nn(Cc3ccc(Cl)cc3)cc1CC2] | UPLC-MS (Method 1): Rₜ = 1.24 min; MS (ESIpos): m/z = 406 [M + H]⁺. | Intermediate 6 and CAS-RN: [430-67-1]; (conditions with HATU) 11.3 mg (17% yield, 91% purity) |
| | 2-[(4-chlorophenyl)methyl]-N-(2,2- | | |
| | difluoroethyl)-8-methyl-4,5-dihydro- | | |
| | 2H-furo[2,3-g]indazole-7- | | |
| | carboxamide | | |
| 66 | 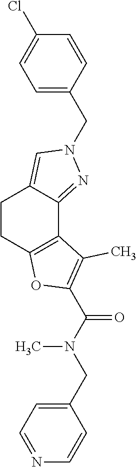 [SMILES: Cc1c(C(=O)N(C)Cc2ccncc2)oc2c1-c1nn(Cc3ccc(Cl)cc3)cc1CC2] | UPLC-MS (Method 1): Rₜ = 1.20 min; MS (ESIpos): m/z = 447 [M + H]⁺. | Intermediate 6 and CAS-RN: [6971- 44-4]; (conditions with HATU) 13.9 mg (20% yield, 97% purity) |
| | 2-[(4-chlorophenyl)methyl]-/,8- | | |
| | dimethyl-N-[(pyridin-4-yl)methyl]- | | |
| | 4,5-dihydro-2H-furo[2,3-g]indazole- | | |
| | 7-carboxamide | | |
| 67 | 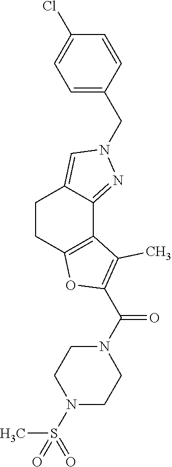 [SMILES: Cc1c(C(=O)N2CCN(S(C)(=O)=O)CC2)oc2c1-c1nn(Cc3ccc(Cl)cc3)cc1CC2] | UPLC-MS (Method 1): Rₜ = 1.17 min; MS (ESIpos): m/z = 489 [M + H]⁺. | Intermediate 6 and CAS-RN: [55276- 43-2]; (conditions with HATU) 14.4 mg (18% yield, 94% purity) |
| | {2-[(4-chlorophenyl)methyl]-8- | | |
| | methyl-4,5-dihydro-2H-furo[2,3- | | |
| | g]indazol-7-yl}[4- | | |
| | (methanesulfonyl)piperazin-1- | | |
| | yl]methanone | | |
| 68 | 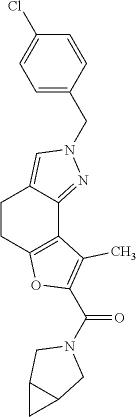 [SMILES: Cc1c(C(=O)N2CC3CC3C2)oc2c1-c1nn(Cc3ccc(Cl)cc3)cc1CC2] | UPLC-MS (Method 1): Rₜ = 1.35 min; MS (ESIpos): m/z = 408 [M + H]⁺. | Intermediate 6 and CAS-RN: [73799-64-1]; (conditions with HATU) 17.3 mg (28% yield, 98% purity) |
| | (3-azabicyclo[3.1.0]hexan-3-yl){2- | | |
| | [(4-chlorophenyl)methyl]-8-methyl- | | |
| | 4,5-dihydro-2H-furo[2,3-g]indazol-7- | | |
| | yl}methanone | | |
| 69 | 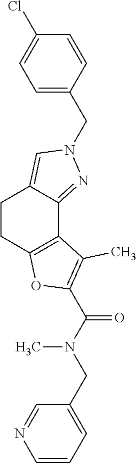 [SMILES: Cc1c(C(=O)N(C)Cc2cccnc2)oc2c1-c1nn(Cc3ccc(Cl)cc3)cc1CC2] | UPLC-MS (Method 1): Rₜ = 1.21 min; MS (ESIpos): m/z = 447 [M + H]⁺. | Intermediate 6 and CAS-RN: [20173-04-0]; (conditions with HATU) 14.9 mg (22% yield, 100% purity) |
| | 2-[(4-chlorophenyl)methyl]-N,8- | | |
| | dimethyl-N-[(pyridin-3-yl)methyl]- | | |
| | 4,5-dihydro-2H-furo[2,3-g]indazole- | | |
| | 7-carboxamide | | |
| 70 | 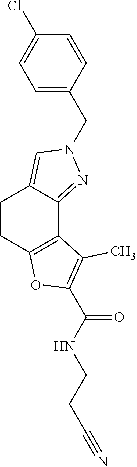 [SMILES: Cc1c(C(=O)NCCC#N)oc2c1-c1nn(Cc3ccc(Cl)cc3)cc1CC2] | UPLC-MS (Method 1): Rₜ = 1.14 min; MS (ESIpos): m/z = 395 [M + H]⁺. | Intermediate 6 and CAS-RN: [151-18-8]; (conditions with HATU) 10.0 mg (17% yield, 100% purity) |
| | 2-[(4-chlorophenyl)methyl]-N-(2- | | |
| | cyanoethyl)-8-methyl-4,5-dihydro- | | |
| | 2H-furo[2,3-g]indazole-7- | | |
| | carboxamide | | |
| 71 | 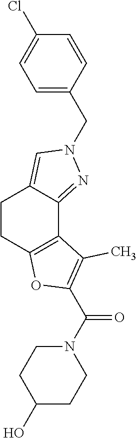 [SMILES: Cc1c(C(=O)N2CCC(O)CC2)oc2c1-c1nn(Cc3ccc(Cl)cc3)cc1CC2] | UPLC-MS (Method 1): Rₜ = 1.10 min; MS (ESIpos): m/z = 426 [M + H]⁺. | Intermediate 6 and CAS-RN: [5382-16-1]; (conditions with HATU) 12.7 mg (20% yield, 100% purity) |
| | {2-[(4-chlorophenyl)methyl]-8- | | |
| | methyl-4,5-dihydro-2H-furo[2,3- | | |
| | g]indazol-7-yl}(4-hydroxypiperidin-1- | | |
| | yl)methanone | | |
| 72 | 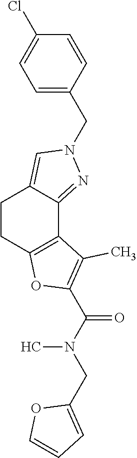 [SMILES: Cc1c(C(=O)N(C)Cc2ccco2)oc2c1-c1nn(Cc3ccc(Cl)cc3)cc1CC2] | UPLC-MS (Method 1): Rₜ = 1.36 min; MS (ESIpos): m/z = 436 [M + H]+ | Intermediate 6 and CAS-RN: [4753-75-7]; (conditions with HATU) 14.9 mg (21% yield, 93% purity) |
| | 2-[(4-chlorophenyl)methyl]-N- | | |
| | [(furan-2-yl)methyl]-N,8-dimethyl- | | |
| | 4,5-dihydro-2H-furo[2,3-g]indazole- | | |
| | 7-carboxamide | | |
| 73 | 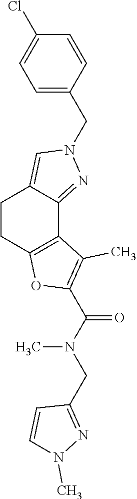 [SMILES: Cc1c(C(=O)N(C)Cc2ccn(C)n2)oc2c1-c1nn(Cc3ccc(Cl)cc3)cc1CC2] | UPLC-MS (Method 1): Rₜ = 1.20 min; MS (ESIpos): m/z = 450 [M + H]⁺. | Intermediate 6 and CAS-RN: [871825-57- 9]; (conditions with HATU) 12.3 mg (18% yield, 99% purity) |
| | 2-[(4-chlorophenyl)methyl]-N,8- | | |
| | dimethyl-N-[(1-methyl-1H-pyrazol-3- | | |
| | yl)methyl]-4,5-dihydro-2H-furo[2,3- | | |
| | g]indazole-7-carboxamide | | |
| 74 | 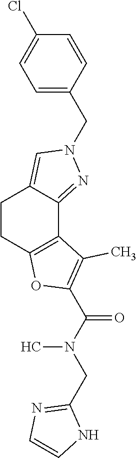 [SMILES: Cc1c(C(=O)N(C)Cc2ncc[nH]2)oc2c1-c1nn(Cc3ccc(Cl)cc3)cc1CC2] | UPLC-MS (Method 1): Rₜ = 1.09 min; MS (ESIpos): m/z = 436 [M + H]⁺. | Intermediate 6 and CAS-RN: [473927-72- 9]; (conditions with HATU) 7.8 mg (11% yield, 95% purity) |
| | 2-[(4-chlorophenyl)methyl]-N-[(1H- | | |
| | imidazol-2-yl)methyl]-N,8-dimethyl- | | |
| | 4,5-dihydro-2H-furo[2,3-g]indazole- | | |
| | 7-carboxamide | | |
| 75 | 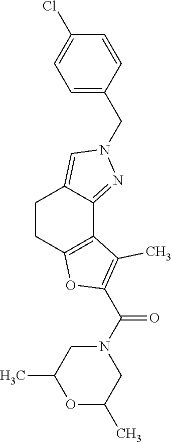 [SMILES: Cc1c(C(=O)N2CC(C)OC(C)C2)oc2c1-c1nn(Cc3ccc(Cl)cc3)cc1CC2] | UPLC-MS (Method 1): Rₜ = 1.33 min; MS (ESIpos): m/z = 440 [M + H]⁺. | Intermediate 6 and CAS-RN: [141-91-3]; (conditions with HATU) 17.9 mg (22% yield, 80% purity) |
| | {2-[(4-chlorophenyl)methyl]-8- | | |
| | methyl-4,5-dihydro-2H-furo[2,3- | | |
| | g]indazol-7-yl}(2,6- | | |
| | dimethylmorpholin-4-yl)methanone | | |
| 76 | 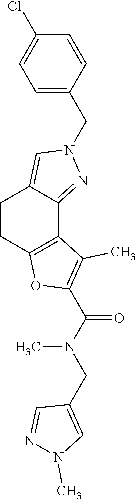 [SMILES: Cc1c(C(=O)N(C)Cc2cnn(C)c2)oc2c1-c1nn(Cc3ccc(Cl)cc3)cc1CC2] | UPLC-MS (Method 1): Rₜ = 1.18 min; MS (ESIpos): m/z = 450 [M + H]⁺. | Intermediate 6 and CAS-RN: [179873-43- 9]; (conditions with HATU) 18.3 mg (27% yield, 99% purity) |
| | 2-[(4-chlorophenyl)methyl]-N,8- | | |
| | dimethyl-N-[(1-methyl-1H-pyrazol-4- | | |
| | yl)methyl]-4,5-dihydro-2H-furo[2,3- | | |
| | g]indazole-7-carboxamide | | |
| 77 | 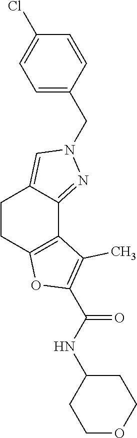 [SMILES: Cc1c(C(=O)NC2CCOCC2)oc2c1-c1nn(Cc3ccc(Cl)cc3)cc1CC2] | UPLC-MS (Method 1): Rₜ = 1.20 min; MS (ESIpos): m/z = 426 [M + H]⁺. | Intermediate 6 and CAS-RN: [38041-19-9]; (conditions with HATU) 14.1 mg (22% yield, 100% purity) |
| | 2-(4-chlorobenzyl)-8-methyl-N- | | |
| | (tetrahydro-2H-pyran-4-yl)-4,5- | | |
| | dihydro-2H-furo[2,3-g]indazole-7- | | |
| | carboxamide | | |
| 78 | 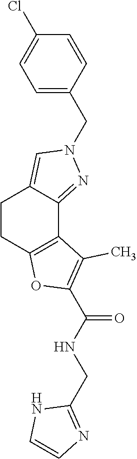 [SMILES: Cc1c(C(=O)NCc2ncc[nH]2)oc2c1-c1nn(Cc3ccc(Cl)cc3)cc1CC2] | UPLC-MS (Method 1): Rₜ = 1.06 min; MS (ESIpos): m/z = 422 [M + H]⁺. | Intermediate 6 and CAS-RN: [53332-80-2]; (conditions with HATU) 3.8 mg (5% yield, 77% purity) |
| | 2-[(4-chlorophenyl)methyl]-N-[(1H- | | |
| | imidazol-2-yl)methyl]-8-methyl-4,5- | | |
| | dihydro-2H-furo[2,3-g]indazole-7- | | |
| | carboxamide | | |
| 79 | 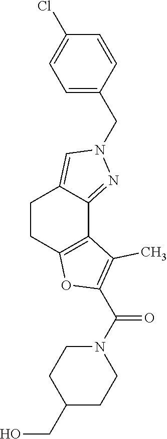 [SMILES: Cc1c(C(=O)N2CCC(CO)CC2)oc2c1-c1nn(Cc3ccc(Cl)cc3)cc1CC2] | UPLC-MS (Method 1): Rₜ = 1.13 min; MS (ESIpos): m/z = 440 [M + H]⁺. | Intermediate 6 and CAS-RN: [6457-49-4]; (conditions with HATU) 15.8 mg (24% yield, 100% purity) |
| | {2-[(4-chlorophenyl)methyl]-8- | | |
| | methyl-4,5-dihydro-2H-furo[2,3- | | |
| | g]indazol-7-yl}[4- | | |
| | (hydroxymethyl)piperidin-1- | | |
| | yl]methanone | | |
| 80 | 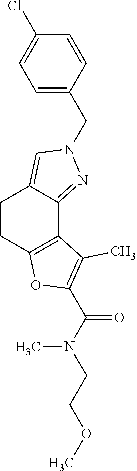 [SMILES: COCCN(C)C(=O)c1oc2c(c1C)-c1nn(Cc3ccc(Cl)cc3)cc1CC2] | UPLC-MS (Method 1): Rₜ = 1.25 min; MS (ESIpos): m/z = 414 [M + H]⁺. | Intermediate 6 and CAS-RN: [38256-93-8]; (conditions with HATU) 18.1 mg (29% yield, 100% purity) |
| | 2-[(4-chlorophenyl)methyl]-N-(2- | | |
| | methoxyethyl)-N,8-dimethyl-4,5- | | |
| | dihydro-2H-furo[2,3-g]indazole-7- | | |
| | carboxamide | | |
| 81 | 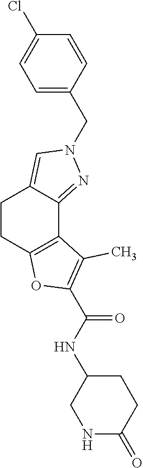 [SMILES: Cc1c(C(=O)NC2CCC(=O)NC2)oc2c1-c1nn(Cc3ccc(Cl)cc3)cc1CC2] | UPLC-MS (Method 1): Rₜ = 1.04 min; MS (ESIpos): m/z = 439 [M + H]⁺. | Intermediate 6 and CAS-RN: [154148-70- 6]; (conditions with HATU) 4.4 mg (6% yield, 92% purity) |
| | 2-[(4-chlorophenyl)methyl]-8-methyl- | | |
| | N-(6-oxopiperidin-3-yl)-4,5-dihydro- | | |
| | 2H-furo[2,3-g]indazole-7- | | |
| | carboxamide | | |
| 82 | 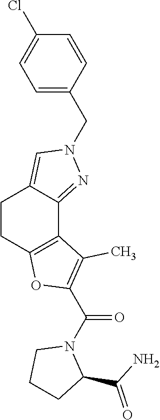 [SMILES: Cc1c(C(=O)N2CCC[C@@H]2C(N)=O)oc2c1-c1nn(Cc3ccc(Cl)cc3)cc1CC2] | UPLC-MS (Method 1): Rₜ = 1.06 min; MS (ESIpos): m/z = 439 [M + H]⁺. | Intermediate 6 and CAS-RN: [62937-45-5]; (conditions with HATU) 11.4 mg (17% yield, 100% purity) |
| | 1-{2-[(4-chlorophenyl)methyl]-8- | | |
| | methyl-4,5-dihydro-2H-furo[2,3- | | |
| | g]indazole-7-carbonyl}-D- | | |
| | prolinamide | | |
| 83 | 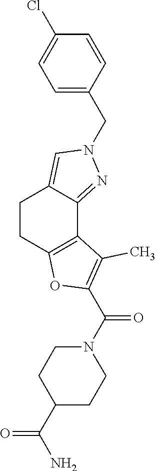 [SMILES: Cc1c(C(=O)N2CCC(C(N)=O)CC2)oc2c1-c1nn(Cc3ccc(Cl)cc3)cc1CC2] | UPLC-MS (Method 1): Rₜ = 1.04 min; MS (ESIpos): m/z = 453 [M + H]⁺. | Intermediate 6 and CAS-RN: [39546-32-2]; (conditions with HATU) 10.4 mg (15% yield, 99% purity) |
| | 1-{2-[(4-chlorophenyl)methyl]-8- | | |
| | methyl-4,5-dihydro-2H-furo[2,3- | | |
| | g]indazole-7-carbonyl}piperidine-4- | | |
| | carboxamide | | |
| 84 | 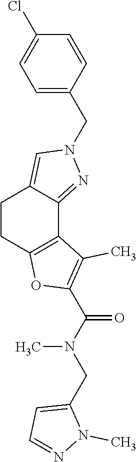 [SMILES: Cc1c(C(=O)N(C)Cc2ccnn2C)oc2c1-c1nn(Cc3ccc(Cl)cc3)cc1CC2] | UPLC-MS (Method 1): Rₜ = 1.20 min; MS (ESIpos): m/z = 450 [M + H]⁺. | Intermediate 6 and CAS-RN: [930111-04- 9]; (conditions with HATU) 18.4 mg (27% yield, 99% purity) |
| | 2-[(4-chlorophenyl)methyl]-N,8- | | |
| | dimethyl-N-[(1-methyl-1H-pyrazol-5- | | |
| | yl)methyl]-4,5-dihydro-2H-furo[2,3- | | |
| | g]indazole-7-carboxamide | | |
| 85 | 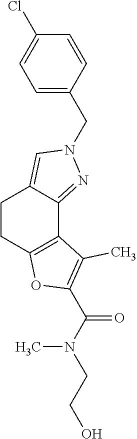 | UPLC-MS (Method 1): Rₜ = 1.09 min; MS (ESIpos): m/z = 400 [M + H]⁺. | Intermediate 6 and CAS-RN: [109-83-1]; (conditions with HATU) 14.4 mg (24% yield, 100% purity) |
| | 2-[(4-chlorophenyl)methyl]-N-(2- | | |
| | hydroxyethyl)-N,8-dimethyl-4,5- | | |
| | dihydro-2H-furo[2,3-g]indazole-7- | | |
| | carboxamide | | |
| 86 | 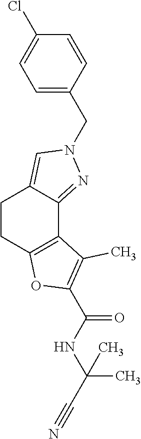 [SMILES: Cc1c(C(=O)NC(C)(C)C#N)oc2c1-c1nn(Cc3ccc(Cl)cc3)cc1CC2] | UPLC-MS (Method 1): Rₜ = 1.25 min; MS (ESIpos): m/z = 409 [M + H]⁺. | Intermediate 6 and CAS-RN: [19355-69-2]; (conditions with HATU) 11.0 mg (14% yield, 78% purity) |
| | 2-[(4-chlorophenyl)methyl]-N-(2- | | |
| | cyanopropan-2-yl)-8-methyl-4,5- | | |
| | dihydro-2H-furo[2,3-g]indazole-7- | | |
| | carboxamide | | |
| 87 | 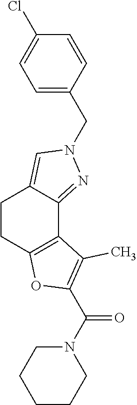 [SMILES: Cc1c(C(=O)N2CCCCC2)oc2c1-c1nn(Cc3ccc(Cl)cc3)cc1CC2] | UPLC-MS (Method 1): Rₜ = 1.37 min; MS (ESIpos): m/z = 410 [M + H]⁺. | Intermediate 6 and CAS-RN: [110-89-4]; (conditions with HATU) 17.6 mg (29% yield, 100% purity) |
| | {2-[(4-chlorophenyl)methyl]-8- | | |
| | methyl-4,5-dihydro-2H-furo[2,3- | | |
| | glindazol-7-yl}(piperidin-1- | | |
| | yl)methanone | | |
| 88 | 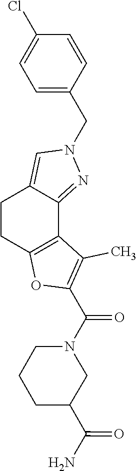 [SMILES: Cc1c(C(=O)N2CCCC(C(N)=O)C2)oc2c1-c1nn(Cc3ccc(Cl)cc3)cc1CC2] | UPLC-MS (Method 1): Rₜ = 1.08 min; MS (ESIpos): m/z = 453 [M + H]⁺. | Intermediate 6 and CAS-RN: [4138-26-5]; (conditions with HATU) 11.7 mg (17% yield, 100% purity) |
| | 1-{2-[(4-chlorophenyl)methyl]-8- | | |
| | methyl-4,5-dihydro-2H-furo[2,3- | | |
| | g]indazole-7-carbonyl}piperidine-3- | | |
| | carboxamide | | |
| 89 | 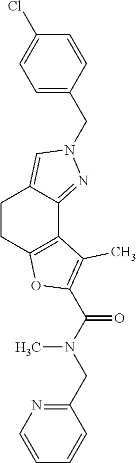 [SMILES: Cc1c(C(=O)N(C)Cc2ccccn2)oc2c1-c1nn(Cc3ccc(Cl)cc3)cc1CC2] | UPLC-MS (Method 1): Rₜ = 1.24 min; MS (ESIpos): m/z = 447 [M + H]⁺. | Intermediate 6 and CAS-RN: [21035-59-6]; (conditions with HATU) 19.4 mg (29% yield, 100% purity) |
| | 2-[(4-chlorophenyl)methyl]-N,8- | | |
| | dimethyl-N-[(pyridin-2-yl)methyl]- | | |
| | 4,5-dihydro-2H-furo[2,3-g]indazole- | | |
| | 7-carboxamide | | |
| 90 | 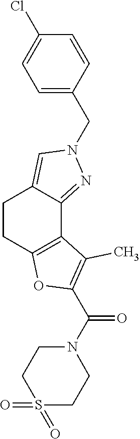 [SMILES: Cc1c(C(=O)N2CCS(=O)(=O)CC2)oc2c1-c1nn(Cc3ccc(Cl)cc3)cc1CC2] | UPLC-MS (Method 1): Rₜ = 1.14 min; MS (ESIpos): m/z = 460 [M + H]⁺. | Intermediate 6 and CAS-RN: [39093-93-1]; (conditions with HATU) 15.1 mg (22% yield, 100% purity) |
| | [2-(4-chlorophenyl)-8-methyl-4,5- | | |
| | dihydro-2H-furo[2,3-g]indazol-7- | | |
| | yl](1,1-dioxidothiomorpholin-4- | | |
| | yl)methanone | | |
| 91 | 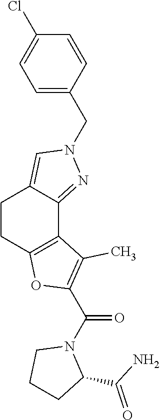 [SMILES: Cc1c(C(=O)N2CCC[C@H]2C(N)=O)oc2c1-c1nn(Cc3ccc(Cl)cc3)cc1CC2] | UPLC-MS (Method 1): Rₜ = 1.06 min; MS (ESIpos): m/z = 439 [M + H]⁺. | Intermediate 6 and CAS-RN: [7531-52-4]; (conditions with HATU) 12.3 mg (19% yield, 100% purity) |
| | 1-{2-[(4-chlorophenyl)methyl]-8- | | |
| | methyl-4,5-dihydro-2H-furo[2,3- | | |
| | g]indazole-7-carbonyl}-L- | | |
| | prolinamide | | |

### Example 92

### 2-[(2-chloro-4-fluorophenyl)methyl]-8-methyl-N-[(2RS)-tetrahydrofuran-2-ylmethyl]-4,5-dihydro-2H-furo[2,3-g]indazole-7-carboxamide

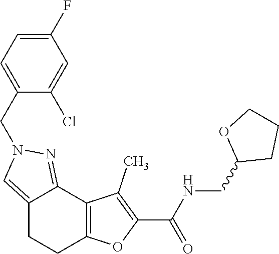 [SMILES: Cc1c(C(=O)NCC2CCCO2)oc2c1-c1nn(Cc3ccc(F)cc3Cl)cc1CC2]

A solution of 2-[(2-chloro-4-fluorophenyl)methyl]-8-methyl-4,5-dihydro-2H-furo[2,3-g]indazole-7-carboxylic acid (Intermediate 7; 1.00 eq., 150 mg, 416 μmol) in DMF (2 mL) was treated with 1-[(2RS)-tetrahydrofuran-2-yl]methanamine (CAS No. [4795-29-3]; 1.2 eq., 51 μL, 500 μmol), HATU (CAS No. [148893-10-1]; 1.50 eq., 237 mg, 624 μmol) and N,N-diisopropylethylamine (CAS No. [7087-68-5]; 3.0 eq., 360 μL, 2.1 mmol) and stirred at rt for three days. The reaction mixture was diluted with EtOAc and the layers separated. The organic layer was washed with water, dried with Na₂SO₂, filtrated and concentrated under reduced pressure. The obtained residue was subjected to column chromatography (Si—NH, SiO₂, hexane/ethyl acetate) to give the title compound (102 mg, 50%).

¹H NMR (400 MHz, DMSO-d6) δ [ppm]: 1.53-1.61 (m, 1H), 1.76-1.90 (m, 3H), 2.44 (s, 3H), 2.84-2.93 (m, 4H), 3.21-3.25 (m, 2H), 3.58-3.64 (m, 1H), 3.73-3.78 (m, 1H), 3.91-3.97 (m, 1H), 5.36 (s, 2H), 7.08 (dd, 1H), 7.24 (dt, 1H), 7.51 (dd, 1H), 7.58 (s, 1H), 7.98 (t, 1H).

UPLC-MS (Method 1): Rₜ=1.31 min; MS (ESIpos): m/z=444/446 [M+H]⁺ (CI isotope pattern).

The enantiomers of the racemic material of Example 92 were separated by chiral preparative HPLC (Instrument Sepiatec: Prep SFC100; Column: Chiralpak IA 5 μm 250×30 mm; Eluent A: CO₂; Eluent B: methanol; Isocratic: 30% B; Flow: 100 mL/min; Temperature: 40° C.; BPR: 150 bar; Detection: UV 254 nm) and analytically characterized by chiral HPLC (Instrument: Agilent 1260, Aurora SFC-Modul; Column: Chiralpak IA 5 μm 100×4.6 mm; Eluent A: CO₂; Eluent B: methanol; Isocratic: 30% B; Flow: 4 mL/min; Temperature: 37° C.; BPR: 100 bar; Detection: UV: 254 nm):

### Example 92-1

27 mg; Rₜ=1.82 min.

### Example 92-2

28 mg; Rₜ=2.21 min.

TABLE 7
The following examples (93 to 94) were prepared in analogy to Example
92 starting from Intermediate 7 and commercially available amines (or their salts).

| | Structure | | |
|---|---|---|---|
| | IUPAC- | | Preparation |
| Example | Name | Analytical Data | Methods |
| 93 | 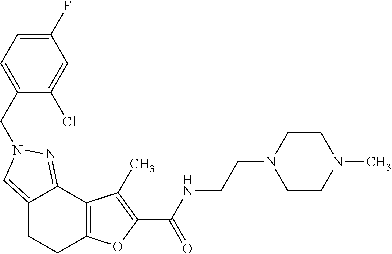 [SMILES: Cc1c(C(=O)NCCN2CCN(C)CC2)oc2c1-c1nn(Cc3ccc(F)cc3Cl)cc1CC2] | ¹H NMR (400 MHz, DMSO-d6) δ [ppm]: 2.13 (s, 3H), 2.26-2.31 (m, 4H), 2.39-2.44 (m, 9H), 2.84-2.91 (m, 4H), 3.27-3.32 (m, 2H), 5.36 (s, 2H), 7.08 (dd, 1H), 7.23 (dt, 1H), 7.51 (dd, 1H), 7.58 (s, 1H), 7.93 (t, 1H). LC-MS (Method A); Rₜ = 1.19 min, m/z = 486/488 [M + H]⁺ (Cl isotope pattern). | Intermediate 7; (conditions with HATU) |
| | 2-[(2-chloro-4-fluorophenyl)methyl]- | | |
| | 8-methyl-N-[2-(4-methylpiperazin-1- | | |
| | yl)ethyl]-4,5-dihydro-2H-furo[2,3- | | |
| | glindazole-7-carboxamide | | |
| 94 | 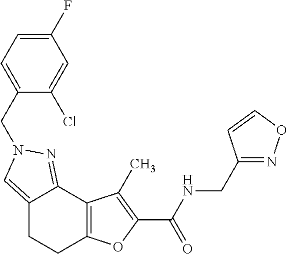 [SMILES: Cc1c(C(=O)NCc2ccon2)oc2c1-c1nn(Cc3ccc(F)cc3Cl)cc1CC2] | ¹H NMR (400 MHz, DMSO-d6) δ [ppm]: 2.46 (s, 3H), 2.85-2.92 (m, 4H), 4.46 (d, 2H), 5.36 (s, 2H), 6.49 (d, 1H), 7.08 (dd, 1H), 7.23 (dt, 1H), 7.51 (dd, 1H), 7.59 (s, 1H), 8.72 (t, 1H), 8.82 (d, 1H). UPLC-MS (Method 1); Rₜ = 1.25 min, m/z = 441/443 [M + H]⁺ (Cl isotope pattern). | Intermediate 7; (conditions with HATU) |
| | 2-[(2-chloro-4-fluorophenyl)methyl]- | | |
| | 8-methyl-N-[(1,2-oxazol-3- | | |
| | yl)methyl]-4,5-dihydro-2H-furo[2,3- | | |
| | glindazole-7-carboxamide | | |

### Example 95

### 2-[(1RS)-1-(2-fluorophenyl)ethyl]-8-methyl-N-[(2S)-tetrahydrofuran-2-ylmethyl]-4,5-dihydro-2H-furo[2,3-g]indazole-7-carboxamide

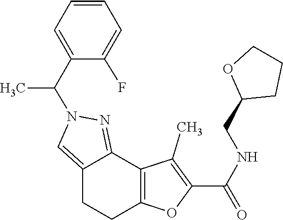 [SMILES: Cc1c(C(=O)NC[C@@H]2CCCO2)oc2c1-c1nn(C(C)c3ccccc3F)cc1CC2]

To a solution of 2-[(1RS)-1-(2-fluorophenyl)ethyl]-8-methyl-4,5-dihydro-2H-furo[2,3-g]indazole-7-carboxylic acid (Intermediate 8; 40.0 mg, 118 μmol) in N,N-dimethylformamide (490 μL, 6.4 mmol; CAS-RN:[68-12-2]) were added 1-[(2S)-tetrahydrofuran-2-yl]methanamine (CAS No. [7175-81-7]; 14.3 mg, 141 μmol), HATU (53.6 mg, 141 μmol; CAS-RN:[148893-10-1]) and N,N-diisopropylethylamine (61 μL, 350 μmol; CAS-RN:[7087-68-5]). The reaction mixture was stirred at rt overnight. The reaction mixture was diluted with acetonitrile (1 mL) and purified by preparative HPLC (Method B, gradient D). The product fractions were pooled and concentrated in vacuo to afford 3.9 mg (7% yield, 89% purity) of the title compound as a mixture of two diastereomers.

¹H NMR (400 MHz, DMSO-d6) b 1.54-1.64 (m, 1H), 1.80 (d, J=7.10 Hz, 3H), 1.82-1.92 (m, 3H), 2.45 (s, 3H), 2.84-2.90 (m, 4H), 3.23 (td, J=6.15, 2.15 Hz, 2H), 3.56-3.66 (m, 1H), 3.70-3.80 (m, 1H), 3.94 (quin, J=6.40 Hz, 1H), 5.80 (q, J=7.01 Hz, 1H), 7.15-7.26 (m, 4H), 7.66 (s, 1H), 7.98 (br t, J=5.83 Hz, 1H).

LC-MS (Method 1): Rₜ=1.34 min; MS (ESIpos): m/z=424 [M+H]⁺.

### Experimental Section—Biological Assays

The in vitro activity of the compounds of the present invention can be demonstrated in the following assay:

cAMP HTRF® Assay for Identification of Cellular GPR84 Antagonists

By using a Homogenous Time-Resolved Fluorescence (HTRF®) based assay (#62AM5PEJ, Cisbio, Condolet, France) the inhibition of the Gi-coupled GPR84 receptor can be detected. CHO-K1 cells stably expressing human GPR84 receptor (purchased from DiscoveRx, now Eurofins) were used and treated with Forskolin (F6886, Sigma, Germany) to stimulate membrane adenylyl cyclases and thereby unspecific cAMP formation. Activation of the Gi-coupled GPR84 by a natural or small molecule agonist (e.g. 6-n-octyl aminouracile, inhouse) results in inhibition of cellular cAMP formation which can be released again by antagonists to this receptor. Detection and quantification of cellular cAMP levels in this HTRF assay is achieved by interaction between a fluorescent cAMP tracer (cAMP-d2) and an Eu-cryptate labelled anti-cAMP antibody. Following excitation at 337 nm this pairing allows for the generation of a fluorescence resonance energy transfer (FRET) between the partners and results in FRET induced emissions at 665 nm and 620 nm, the latter representing background signal by Eu-cryptate labelled anti-cAMP antibody. Maximal signal is obtained in the absence of any cellular cAMP (no competition for the binding of the tracer to the antibody). Given the combination of the Gi coupling properties of GPR84 and the competitive nature of the detection system agonist treatment should result in an increase in the HTRF signal due to lowered cAMP levels. Any signal decrease in the presence of Forskolin, agonist and compound are indicative of antagonist mediated abrogation of GPR84 signalling.

For the assay, frozen aliquots of CHO-K1 cells expressing hGPR84 (prepared by acCELLerate, Hamburg, Germany) were thawed and a cell suspension (1.67E+06 cells/mL) in assay media (Ham's F12 Nutrient Mix, Thermo Fisher Scientific, Waltham, USA; 5% fetal calf serum, Biomol, Hamburg, Germany) containing cAMP-d2 (dilution 1:20, supplied with the kit #62AM5PEJ, Cisbio, Condolet, France) was prepared. After recovery of cells for 20 minutes at 37° C., 3 μL/well cell suspension including cAMP-d2 were added to a pre-dispensed assay plate (Greiner Bio-One, Kremsmuenster, Austria) containing 50 nl/well test compound in 100% DMSO or 100% DMSO as control. This was followed by a 30 minute incubation step at room temperature. The stimulation time was started by addition of 2 μL/well assay media containing 2.5×EC₈₀ agonist 6-OAU and 2.5×EC₉₀ Forskolin (negative control: 2.5×EC₉₀ Forskolin in assay media) and was continued for 30 minutes at room temperature. The reaction was stopped by addition of 3 μL/well lysis buffer containing cAMP Eu-Cryptate antibody (dilution 1:20) (both supplied with the kit #62AM5PEJ, Cisbio, Condolet, France). To enable complete lysis, plates were incubated for 60 minutes at room temperature before measurement in an HTRF reader, e.g. a PHERAstar (BMG Labtech, Ortenberg, Germany).

From the fluorescence emissions at 665 nm (FRET) and at 620 nm (background signal of Eu-cryptate) the ratio (emission at 665 nm divided by emission at 620 nm×10000) was calculated and the data were normalized (reaction without test compound, only 100% DMSO=0% inhibition; all other assay components except agonist=100% inhibition). For dose response testing on the same microtiter plate, compounds were tested at 11 different concentrations in the range of 20 μM to 0.07 nM (20 μM, 5.7 μM, 1.6 μM, 0.47 μM, 0.13 μM, 38 nM, 11 nM, 3.1 nM, 0.89 nM, 0.25 and 0.07 nM; dilution series prepared before the assay at the level of the 100-fold conc. stock solutions by serial 1:3.5 dilutions in 100% DMSO) in duplicate values for each concentration. IC₅₀ values were calculated by 4-parameter fitting using a commercial software package (Genedata Screener, Basel, Switzerland).

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

TABLE 8
Potency in GPR84 cAMP HTRF ® assay, the
potency is given as IC₅₀ [μM].

| Example | GPR84 IC₅₀ [μM] |
|---|---|
| 1 | 0.018 |
| 2 | 0.007 |
| 3 | 0.14 |
| 4 | 0.023 |
| 5 | 0.009 |
| 6 | 2.22 |
| 7 | 0.68 |
| 7-1 | 0.72 |
| 7-2 | 0.63 |
| 7-3 | 1.18 |
| 8 | 0.24 |
| 9 | 0.046 |
| 9-1 | 0.015 |
| 9-2 | 0.060 |
| 10 | 0.042 |
| 11 | 0.23 |
| 12 | 0.30 |
| 13 | 0.36 |
| 14 | 1.15 |
| 15 | 0.074 |
| 15-1 | 0.088 |
| 15-2 | 0.55 |
| 16 | 0.10 |
| 17 | 0.32 |
| 18 | 1.00 |
| 19 | 0.78 |
| 20 | 1.54 |
| 21 | 0.058 |
| 21-1 | 0.52 |
| 21-2 | 0.089 |
| 22 | 0.34 |
| 23 | 0.30 |
| 24 | 1.05 |
| 25 | 1.37 |
| 26 | 1.63 |
| 27 | 0.15 |
| 28 | 1.01 |
| 29 | 8.21 |
| 30 | 0.50 |
| 31 | 2.53 |
| 32 | 2.46 |
| 33 | 3.31 |
| 34 | 1.14 |
| 35 | 3.00 |
| 36 | 1.19 |
| 37 | 0.18 |
| 38 | 0.21 |
| 39 | 0.28 |
| 40 | 0.41 |
| 41 | 0.52 |
| 42 | 0.72 |
| 43 | 0.80 |
| 44 | 0.89 |
| 45 | 1.43 |
| 46 | 1.65 |
| 47 | 1.87 |
| 48 | 2.13 |
| 49 | 2.59 |
| 50 | 2.69 |
| 51 | 2.72 |
| 52 | 2.96 |
| 53 | 3.04 |
| 54 | 3.39 |
| 55 | 4.18 |
| 56 | 4.88 |
| 57 | 5.04 |
| 58 | 5.22 |
| 59 | 5.28 |
| 60 | 5.64 |
| 61 | 6.02 |
| 62 | 6.10 |
| 63 | 6.15 |
| 64 | 6.68 |
| 65 | 7.45 |
| 66 | 7.59 |
| 67 | 7.83 |
| 68 | 7.97 |
| 69 | 9.40 |
| 70 | 9.50 |
| 71 | 9.57 |
| 72 | 9.96 |
| 73 | 10.3 |
| 74 | 10.8 |
| 75 | 12.3 |
| 76 | 13.0 |
| 77 | 13.8 |
| 78 | 14.3 |
| 79 | 15.5 |
| 80 | 15.9 |
| 81 | 16.1 |
| 82 | 16.4 |
| 83 | 16.4 |
| 84 | 16.4 |
| 85 | 17.0 |
| 86 | 17.1 |
| 87 | 17.1 |
| 88 | 17.3 |
| 89 | 17.4 |
| 90 | 18.9 |
| 91 | 20 |
| 92 | 0.16 |
| 92-1 | 0.12 |
| 92-2 | 0.34 |
| 93 | 0.82 |
| 94 | 0.28 |
| 95 | 0.75 |

1. A compound of general formula (I): 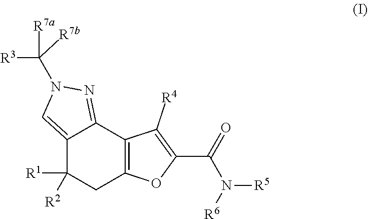
in which:
R¹ represents hydrogen, C₁-C₄-alkyl or C₁-C₄-haloalkyl;
R² represents hydrogen, C₁-C₄-alkyl or C₁-C₄-haloalkyl; or
R¹ and R² together with the carbon atom to which they are attached form a 3- to 6-membered cycloalkyl or heterocycloalkyl ring;
R³ represents phenyl, which is optionally substituted, one or more times, independently of each other, with R⁸;
R⁴ represents hydrogen, C₁-C₄-alkyl, C₁-C₄-haloalkyl or C₃-C₆-cycloalkyl;
R⁵ represents hydrogen or C₁-C₄-alkyl;
R⁶ represent hydrogen, C₁-C₆-alkyl, wherein said C₁-C₆-alkyl group is optionally substituted with R¹⁴, C₂-C₄-hydroxyalkyl, (C₁-C₄-alkoxy)-(C₂-C₄-alkyl)-, C₃-C₆-cycloalkyl, C₁-C₄-haloalkyl, C₃-C₆-halocycloalkyl, 3- to 6-membered heterocycloalkyl, heterospirocycloalkyl, phenyl, heteroaryl, heterocycloalkyl fused with phenyl or heteroaryl, 3- to 7-membered heterocycloalkyl-(C₁-C₃-alkyl)-, heterospirocycloalkyl-(C₁-C₃-alkyl)-, (heterocycloalkyl fused with phenyl or heteroaryl)-(C₁-C₃-alkyl)-, phenyl-(C₁-C₃-alkyl)- or heteroaryl-(C₁-C₃-alkyl)-, wherein said 3- to 6-membered or 3- to 7-membered heterocycloalkyl, heterospirocycloalkyl, heterocycloalkyl fused with phenyl or heteroaryl, phenyl or heteroaryl groups are optionally substituted, one or more times, independently of each other, with R⁹, or
R⁵ and R⁶ together with the nitrogen atom to which they are attached form a 4- to 7-membered nitrogen containing heterocyclic ring, optionally containing one additional heteroatom or heteroatom containing group selected from O, NH, S, and SO₂, and which may be optionally substituted, one or more times, independently of each other, with R⁹, or a 1,2,3,4-tetrahydroisoquinoline, or a 3-azabicyclo[3.1.0]hexane;
R^{7a} represents hydrogen or C₁-C₄-alkyl;
R^{7b} represents hydrogen or C₁-C₄-alkyl;
R⁸ represents halogen, cyano, C₁-C₄-alkyl, C₁-C₄-haloalkyl, C₁-C₃-alkoxy, C₁-C₃-haloalkoxy, C₃-C₆-cycloalkyl, C₃-C₆-cycloalkyl-(C₁-C₃-alkyl)-, R¹³—(C═O)—, R¹⁰—O—(C═O)—, R¹¹—NH—(C═O)—, or R¹²—(SOₓ)—;
R⁹ represents halogen, cyano, C₁-C₄-alkyl, C₁-C₄-haloalkyl, H₂N—C₁-C₄-alkyl, C₁-C₃-alkoxy, C₁-C₃-haloalkoxy, C₃-C₆-cycloalkyl, R¹⁵—(C═O)—, R¹⁰—O—(C═O)—, R^{11a}R^{11b}N—, R¹⁵—(C═O)—R^{11a}N—, R^{11a}R^{11b}N—(C═O)—, R¹²—(SOₓ)—; oxo, 5- to 6-membered heterocycloalkyl-, 5- to 6-membered heterocycloalkyl-(C₁-C₃-alkyl)-, benzyl, phenyl, or heteroaryl, wherein said phenyl or heteroaryl group is optionally substituted, one or more times, independently of each other, with halogen, C₁-C₄-alkyl, C₁-C₄-haloalkyl, C₁-C₃-alkoxy, or C₁-C₃-haloalkoxy;
R¹⁰ represents hydrogen, C₁-C₄-alkyl, or benzyl;
R^{11a} represents hydrogen or C₁-C₃-alkyl;
R^{11b} represents hydrogen, C₁-C₃-alkyl, C₃-C₆-cycloalkyl, methoxyethyl, or
R^{11a} and R^{11b} together with the nitrogen atom to which they are attached form a 5- to 6-membered nitrogen containing heterocyclic ring, optionally containing one additional heteroatom selected from O, NH, and S, and which may be optionally substituted, with (C₁-C₃-alkyl)-(C═O)—;
R¹² represents C₁-C₄-alkyl or phenyl;
R¹³ represents C₁-C₄-alkyl, C₁-C₄-haloalkyl, (C₁-C₄-alkoxy)-(C₁-C₄-alkyl)-, C₁-C₄-alkyl-(C═O)—, C₃-C₆-cycloalkyl, or phenyl, wherein said C₃-C₆-cycloalkyl group is optionally substituted with C₁-C₄-alkyl or hydroxy and said phenyl group is optionally substituted, one or more times, independently of each other, with halogen, C₁-C₄-alkyl, C₁-C₄-haloalkyl, C₁-C₃-alkoxy, or C₁-C₃-haloalkoxy;
R¹⁴ represents cyano, R^{11a}R^{11b}N—, R^{11a}R^{11b}N—(C═O)—, or R¹²—(SOₓ)—;
R¹⁵ represents C₁-C₃-alkyl or phenyl;
x represents 0, 1, or 2;
or a stereoisomer, a tautomer, an N-oxide, a hydrate, a solvate, or a salt thereof, or a mixture of same.

2. The compound according to claim 1, wherein
R¹ represents hydrogen or C₁-C₃-alkyl;
R² represents hydrogen or C₁-C₃-alkyl;
R³ represents phenyl, which is optionally substituted, one or two times, independently of each other, with R⁸;
R⁴ represents hydrogen, C₁-C₃-alkyl;
R⁵ represents hydrogen or C₁-C₃-alkyl;
R⁶ represent C₁-C₆-alkyl, wherein said C₁-C₆-alkyl group is optionally substituted with R¹⁴, C₂-C₄-hydroxyalkyl, (C₁-C₃-alkoxy)-(C₂-C₃-alkyl)-, C₃-C₆-cycloalkyl, C₁-C₃-haloalkyl, 3- to 6-membered heterocycloalkyl, phenyl, heteroaryl, heterocycloalkyl fused with phenyl, 3- to 7-membered heterocycloalkyl-(C₁-C₃-alkyl)-, (heterocycloalkyl fused with phenyl)-(C₁-C₃-alkyl)-, phenyl-(C₁-C₃-alkyl)- or heteroaryl-(C₁-C₃-alkyl)-, wherein said 3- to 6-membered or 3- to 7-membered heterocycloalkyl, heterocycloalkyl fused with phenyl, phenyl or heteroaryl groups are optionally substituted, one or more times, independently of each other, with R⁹, or
R⁵ and R⁶ together with the nitrogen atom to which they are attached form a 4- to 7-membered nitrogen containing heterocyclic ring, optionally containing one additional heteroatom or heteroatom containing group selected from O, NH, S, and SO₂, and which may be optionally substituted, one or more times, independently of each other, with R⁹, or a 1,2,3,4-tetrahydroisoquinoline, or a 3-azabicyclo[3.1.0]hexane;
R^{7a} represents hydrogen or C₁-C₃-alkyl;
R^{7b} represents hydrogen or C₁-C₃-alkyl;
R⁸ represents halogen, C₁-C₃-alkyl;
R⁹ represents halogen, C₁-C₄-alkyl, C₁-C₃-haloalkyl, C₁-C₃-alkoxy, R¹⁵—(C═O)—, R¹⁰—O—(C═O)—, R^{11a}R^{11b}N—, R¹⁵—(C═O)—R^{11a}N—, R^{11a}R^{11b}N—(C═O)—, R¹²—(SOₓ)—; oxo, benzyl, phenyl, or heteroaryl, wherein said phenyl or heteroaryl group is optionally substituted, one or more times, independently of each other, with halogen, C₁-C₃-alkyl, or C₁-C₃-alkoxy;
R¹⁰ represents C₁-C₄-alkyl, or benzyl;
R^{11a} represents hydrogen or C₁-C₃-alkyl;
R^{11b} represents hydrogen, C₁-C₃-alkyl, C₃-C₆-cycloalkyl, methoxyethyl, or
R^{11a} and R^{11b} together with the nitrogen atom to which they are attached form a 5- to 6-membered nitrogen containing heterocyclic ring, optionally containing one additional heteroatom selected from O, NH, and S, and which may be optionally substituted, with (C₁-C₃-alkyl)-(C═O)—;
R¹² represents C₁-C₃-alkyl;
R¹⁴ represents cyano, R^{11a}R^{11b}N—, R^{11a}R^{11b}N—(C═O)—, or R¹²—(SOₓ)—;
R¹⁵ represents C₁-C₃-alkyl or phenyl;
x represents 0, 1, or 2;
or a stereoisomer, a tautomer, an N-oxide, a hydrate, a solvate, or a salt thereof, or a mixture of same.

3. The compound according to claim 1, wherein:
R¹ represents hydrogen;
R² represents hydrogen;
R³ represents phenyl, which is optionally substituted, one or two times, independently of each other, with R⁸;
R⁴ represents methyl;
R⁵ represents hydrogen or methyl;
R⁶ represent C₁-C₅-alkyl, wherein said C₁-C₅-alkyl group is optionally substituted with R¹⁴, C₂-C₄-hydroxyalkyl, (C₁-C₃-alkoxy)-(C₂-C₃-alkyl)-, C₃-C₅-cycloalkyl, difluoroethyl, 6-membered heterocycloalkyl, phenyl, heterocycloalkyl fused with phenyl, 4- to 7-membered heterocycloalkyl-(C₁-C₃-alkyl)-, (heterocycloalkyl fused with phenyl)-methyl, phenyl-(C₁-C₃-alkyl)- or heteroaryl-(C₁-C₂-alkyl)-, wherein said 6-membered or 4- to 7-membered heterocycloalkyl, phenyl or heteroaryl groups are optionally substituted, one or more times, independently of each other, with R⁹, or
R⁵ and R⁶ together with the nitrogen atom to which they are attached form a 4- to 7-membered nitrogen containing heterocyclic ring, optionally containing one additional heteroatom or heteroatom containing group selected from O, NH, and SO₂, and which may be optionally substituted, one or two times, with R⁹, or a 1,2,3,4-tetrahydroisoquinoline, or a 3-azabicyclo[3.1.0]hexane;
R^{7a} represents hydrogen;
R^{7b} represents hydrogen or methyl;
R⁸ represents fluoro, chloro or methyl;
R⁹ represents fluoro, chloro, bromo, C₁-C₃-alkyl, trifluoromethyl, C₁-C₃-alkoxy, R¹⁵—(C═O)—, R¹⁰—O—(C═O)—, R^{11a}R^{11b}N—, CH₃—(C═O)—HN—, R^{11a}R^{11b}N—(C═O)—, R¹²—(SOₓ)—, oxo, benzyl, or phenyl, wherein said phenyl group is optionally substituted, one or two times, independently of each other, with fluoro, methyl, or methoxy;
R¹⁰ represents ethyl or tert.-butyl;
R^{11a} represents hydrogen, methyl or ethyl;
R^{11b} represents hydrogen, methyl, ethyl, cyclopropyl, cyclohexyl, or methoxyethyl, or
R^{11a} and R^{11b} together with the nitrogen atom to which they are attached form a 5- to 6-membered nitrogen containing heterocyclic ring, optionally containing one additional heteroatom selected from O, and NH, and which may be optionally substituted, with methyl-(C═O)—;
R¹² represents methyl;
R¹⁴ represents cyano, R^{11a}R^{11b}N—, R^{11a}R^{11b}N—(C═O)—, or R¹²—(SOₓ)—;
R¹⁵ represents methyl or phenyl;
x represents 0, 1, or 2;
or a stereoisomer, a tautomer, an N-oxide, a hydrate, a solvate, or a salt thereof, or a mixture of same.

4. The compound according to claim 1, which is selected from the group consisting of:
8-methyl-2-(4-methylbenzyl)-N-[(2S)-tetrahydrofuran-2-ylmethyl]-4,5-dihydro-2H-furo[2,3-g]indazole-7-carboxamide,
2-(2-chlorobenzyl)-8-methyl-N-(4-methylbenzyl)-4,5-dihydro-2H-furo[2,3-g]indazole-7-carboxamide,
2-(2-chlorobenzyl)-8-methyl-N-[2-(4-methylpiperazin-1-yl)ethyl]-4,5-dihydro-2H-furo[2,3-g]indazole-7-carboxamide,
2-(2-chlorobenzyl)-8-methyl-N-[2-(pyrrolidin-1-yl)ethyl]-4,5-dihydro-2H-furo[2,3-g]indazole-7-carboxamide,
2-(2-chlorobenzyl)-8-methyl-N-[2-(piperidin-1-yl)ethyl]-4,5-dihydro-2H-furo[2,3-g]indazole-7-carboxamide,
N-[2-(azetidin-1-yl)ethyl]-2-(2-chlorobenzyl)-8-methyl-4,5-dihydro-2H-furo[2,3-g]indazole-7-carboxamide,
2-(3-chlorobenzyl)-8-methyl-N-[(2RS)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-[(2R*)-tetrahydrofuran-2-ylmethyl]-4,5-dihydro-2H-furo[2,3-g]indazole-7-carboxamide,
2-(3-chlorobenzyl)-8-methyl-N-(4-methylbenzyl)-4,5-dihydro-2H-furo[2,3-g]indazole-7-carboxamide,
2-(3-chlorobenzyl)-8-methyl-N-[2-(4-methylpiperazin-1-yl)ethyl]-4,5-dihydro-2H-furo[2,3-g]indazole-7-carboxamide,
2-(3-chlorobenzyl)-8-methyl-N-[2-(pyrrolidin-1-yl)ethyl]-4,5-dihydro-2H-furo[2,3-g]indazole-7-carboxamide,
2-(3-chlorobenzyl)-8-methyl-N-[2-(piperidin-1-yl)ethyl]-4,5-dihydro-2H-furo[2,3-g]indazole-7-carboxamide,
N-[(2R)-1,4-dioxan-2-ylmethyl]-8-methyl-2-(4-methylbenzyl)-4,5-dihydro-2H-furo[2,3-g]indazole-7-carboxamide,
N-[2-(azetidin-1-yl)ethyl]-2-(3-chlorobenzyl)-8-methyl-4,5-dihydro-2H-furo[2,3-g]indazole-7-carboxamide,
2-(4-chlorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(4-chlorobenzyl)-8-methyl-N-[(2R*)-tetrahydrofuran-2-ylmethyl]-4,5-dihydro-2H-furo[2,3-g]indazole-7-carboxamide,
2-(4-chlorobenzyl)-8-methyl-N-(4-methylbenzyl)-4,5-dihydro-2H-furo[2,3-g]indazole-7-carboxamide,
2-(4-chlorobenzyl)-8-methyl-N-[2-(4-methylpiperazin-1-yl)ethyl]-4,5-dihydro-2H-furo[2,3-g]indazole-7-carboxamide,
2-(4-chlorobenzyl)-8-methyl-N-[2-(pyrrolidin-1-yl)ethyl]-4,5-dihydro-2H-furo[2,3-g]indazole-7-carboxamide,
2-(4-chlorobenzyl)-8-methyl-N-[2-(piperidin-1-yl)ethyl]-4,5-dihydro-2H-furo[2,3-g]indazole-7-carboxamide,
N-[2-(azetidin-1-yl)ethyl]-2-(4-chlorobenzyl)-8-methyl-4,5-dihydro-2H-furo[2,3-g]indazole-7-carboxamide,
2-(4-chlorobenzyl)-8-methyl-N-(1,2-oxazol-3-ylmethyl)-4,5-dihydro-2H-furo[2,3-g]indazole-7-carboxamide,
2-(4-chlorobenzyl)-8-methyl-N-(1,2-oxazol-5-ylmethyl)-4,5-dihydro-2H-furo[2,3-g]indazole-7-carboxamide,
N-[2-(4-acetylpiperazin-1-yl)-2-oxoethyl]-2-(4-chlorobenzyl)-8-methyl-4,5-dihydro-2H-furo[2,3-g]indazole-7-carboxamide,
8-methyl-2-(4-methylbenzyl)-N-(1,2-oxazol-3-ylmethyl)-4,5-dihydro-2H-furo[2,3-g]indazole-7-carboxamide,
2-(4-chlorobenzyl)-N-[2-(ethylamino)-2-oxoethyl]-8-methyl-4,5-dihydro-2H-furo[2,3-g]indazole-7-carboxamide,
2-(4-chlorobenzyl)-N-[2-(diethylamino)-2-oxoethyl]-8-methyl-4,5-dihydro-2H-furo[2,3-g]indazole-7-carboxamide,
2-(4-chlorobenzyl)-N-{2-[(2-methoxyethyl)amino]-2-oxoethyl}-8-methyl-4,5-dihydro-2H-furo[2,3-g]indazole-7-carboxamide,
2-(4-chlorobenzyl)-N-[2-(cyclopropylamino)-2-oxoethyl]-8-methyl-4,5-dihydro-2H-furo[2,3-g]indazole-7-carboxamide,
2-(4-chlorobenzyl)-8-methyl-N-[2-oxo-2-(pyrrolidin-1-yl)ethyl]-4,5-dihydro-2H-furo[2,3-g]indazole-7-carboxamide,
2-(4-chlorobenzyl)-8-methyl-N-[2-(morpholin-4-yl)-2-oxoethyl]-4,5-dihydro-2H-furo[2,3-g]indazole-7-carboxamide,
2-(4-chlorobenzyl)-N-[2-(dimethylamino)-2-oxoethyl]-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](piperidin-1-yl)methanone,
2-(4-chlorobenzyl)-N-(2-hydroxyethyl)-8-methyl-4,5-dihydro-2H-furo[2,3-g]indazole-7-carboxamide,
[2-(4-chlorobenzyl)-8-methyl-4,5-dihydro-2H-furo[2,3-g]indazol-7-yl](2,6-dimethylmorpholin-4-yl)methanone,
8-methyl-2-(2-methylbenzyl)-N-[(2S)-tetrahydrofuran-2-ylmethyl]-4,5-dihydro-2H-furo[2,3-g]indazole-7-carboxamide,
[2-(4-chlorobenzyl)-8-methyl-4,5-dihydro-2H-furo[2,3-g]indazol-7-yl](4-hydroxypiperidin-1-yl)methanone,
2-(4-chlorobenzyl)-8-methyl-N-(2-phenylethyl)-4,5-dihydro-2H-furo[2,3-g]indazole-7-carboxamide,
2-(4-chlorobenzyl)-N-(2-hydroxyethyl)-N,8-dimethyl-4,5-dihydro-2H-furo[2,3-g]indazole-7-carboxamide,
2-(4-chlorobenzyl)-N-(2-methoxyethyl)-8-methyl-4,5-dihydro-2H-furo[2,3-g]indazole-7-carboxamide,
2-(4-chlorobenzyl)-N-(3-hydroxypropyl)-8-methyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}-L-prolinamide,
1-(4-{[2-(4-chlorobenzyl)-8-methyl-4,5-dihydro-2H-furo[2,3-g]indazol-7-yl]carbonyl}piperazin-1-yl)ethanone,
2-(4-chlorobenzyl)-N-(cyclopropylmethyl)-8-methyl-4,5-dihydro-2H-furo[2,3-g]indazole-7-carboxamide,
2-(4-chlorobenzyl)-8-methyl-N-[2-(morpholin-4-yl)ethyl]-4,5-dihydro-2H-furo[2,3-g]indazole-7-carboxamide,
2-(4-chlorobenzyl)-8-methyl-N-(pyridin-2-ylmethyl)-4,5-dihydro-2H-furo[2,3-g]indazole-7-carboxamide,
N-[(2R)-1,4-dioxan-2-ylmethyl]-8-methyl-2-(2-methylbenzyl)-4,5-dihydro-2H-furo[2,3-g]indazole-7-carboxamide,
2-(4-chlorobenzyl)-8-methyl-N-(pyridin-4-ylmethyl)-4,5-dihydro-2H-furo[2,3-g]indazole-7-carboxamide,
N-(2-amino-2-oxoethyl)-2-(4-chlorobenzyl)-8-methyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}piperidine-3-carboxamide,
[2-(4-chlorobenzyl)-8-methyl-4,5-dihydro-2H-furo[2,3-g]indazol-7-yl](3,4-dihydroisoquinolin-2(1H)-yl)methanone,
2-(4-chlorobenzyl)-N-(2-cyanoethyl)-8-methyl-4,5-dihydro-2H-furo[2,3-g]indazole-7-carboxamide,
2-(4-chlorobenzyl)-N-(2-furylmethyl)-N,8-dimethyl-4,5-dihydro-2H-furo[2,3-g]indazole-7-carboxamide,
2-(4-chlorobenzyl)-N,8-dimethyl-N-(pyridin-3-ylmethyl)-4,5-dihydro-2H-furo[2,3-g]indazole-7-carboxamide,
2-(4-chlorobenzyl)-N-(2-cyanopropan-2-yl)-8-methyl-4,5-dihydro-2H-furo[2,3-g]indazole-7-carboxamide,
2-(4-chlorobenzyl)-N,8-dimethyl-N-(pyridin-4-ylmethyl)-4,5-dihydro-2H-furo[2,3-g]indazole-7-carboxamide,
N-(4-acetamidobenzyl)-2-(4-chlorobenzyl)-8-methyl-4,5-dihydro-2H-furo[2,3-g]indazole-7-carboxamide,
tert-butyl (2RS)-2-[({[8-methyl-2-(2-methylbenzyl)-4,5-dihydro-2H-furo[2,3-g]indazol-7-yl]carbonyl}amino)methyl]pyrrolidine-1-carboxylate,
[2-(4-chlorobenzyl)-8-methyl-4,5-dihydro-2H-furo[2,3-g]indazol-7-yl](1,1-dioxidothiomorpholin-4-yl)methanone,
2-(4-chlorobenzyl)-8-methyl-N-(tetrahydro-2H-pyran-4-ylmethyl)-4,5-dihydro-2H-furo[2,3-g]indazole-7-carboxamide,
2-(4-chlorobenzyl)-8-methyl-N-(tetrahydro-2H-pyran-4-yl)-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}piperidine-4-carboxamide,
2-(4-chlorobenzyl)-8-methyl-N-[(1-methyl-1H-pyrrol-2-yl)methyl]-4,5-dihydro-2H-furo[2,3-g]indazole-7-carboxamide,
2-(4-chlorobenzyl)-8-methyl-N-(1,3-thiazol-2-ylmethyl)-4,5-dihydro-2H-furo[2,3-g]indazole-7-carboxamide,
2-(4-chlorobenzyl)-8-methyl-N-[2-(methylsulfinyl)ethyl]-4,5-dihydro-2H-furo[2,3-g]indazole-7-carboxamide,
2-(4-chlorobenzyl)-N,8-dimethyl-N-(pyridin-2-ylmethyl)-4,5-dihydro-2H-furo[2,3-g]indazole-7-carboxamide,
2-(4-chlorobenzyl)-N-(2-methoxyethyl)-N,8-dimethyl-4,5-dihydro-2H-furo[2,3-g]indazole-7-carboxamide,
1-{[2-(4-chlorobenzyl)-8-methyl-4,5-dihydro-2H-furo[2,3-g]indazol-7-yl]carbonyl}-D-prolinamide,
8-methyl-2-(2-methylbenzyl)-N-[(2RS)-pyrrolidin-2-ylmethyl]-4,5-dihydro-2H-furo[2,3-g]indazole-7-carboxamide,
[2-(4-chlorobenzyl)-8-methyl-4,5-dihydro-2H-furo[2,3-g]indazol-7-yl][4-(hydroxymethyl)piperidin-1-yl]methanone,
2-(4-chlorobenzyl)-N-(2,2-difluoroethyl)-8-methyl-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
2-(4-chlorobenzyl)-8-methyl-N-[(1-methyl-1H-pyrazol-3-yl)methyl]-4,5-dihydro-2H-furo[2,3-g]indazole-7-carboxamide,
8-methyl-2-(2-methylbenzyl)-N-[(2R*)-pyrrolidin-2-ylmethyl]-4,5-dihydro-2H-furo[2,3-g]indazole-7-carboxamide,
2-(4-chlorobenzyl)-8-methyl-N-[(1-methyl-1H-pyrazol-5-yl)methyl]-4,5-dihydro-2H-furo[2,3-g]indazole-7-carboxamide,
2-(4-chlorobenzyl)-N,8-dimethyl-N-[(1-methyl-1H-pyrazol-3-yl)methyl]-4,5-dihydro-2H-furo[2,3-g]indazole-7-carboxamide,
2-(4-chlorobenzyl)-N,8-dimethyl-N-[(1-methyl-1H-pyrazol-5-yl)methyl]-4,5-dihydro-2H-furo[2,3-g]indazole-7-carboxamide,
2-(4-chlorobenzyl)-8-methyl-N-(1H-pyrazol-3-ylmethyl)-4,5-dihydro-2H-furo[2,3-g]indazole-7-carboxamide,
2-(4-chlorobenzyl)-N-(1H-imidazol-2-ylmethyl)-8-methyl-4,5-dihydro-2H-furo[2,3-g]indazole-7-carboxamide,
2-(4-chlorobenzyl)-8-methyl-N-[(1-methyl-1H-imidazol-5-yl)methyl]-4,5-dihydro-2H-furo[2,3-g]indazole-7-carboxamide,
N-(4-carbamoylbenzyl)-2-(4-chlorobenzyl)-8-methyl-4,5-dihydro-2H-furo[2,3-g]indazole-7-carboxamide,
8-methyl-2-(2-methylbenzyl)-N-(1,2-oxazol-3-ylmethyl)-4,5-dihydro-2H-furo[2,3-g]indazole-7-carboxamide,
[2-(4-chlorobenzyl)-8-methyl-4,5-dihydro-2H-furo[2,3-g]indazol-7-yl][4-(methylsulfonyl)piperazin-1-yl]methanone,
2-(4-chlorobenzyl)-N,8-dimethyl-N-[(1-methyl-1H-pyrazol-4-yl)methyl]-4,5-dihydro-2H-furo[2,3-g]indazole-7-carboxamide,
2-(4-chlorobenzyl)-8-methyl-N-[(1-methyl-1H-pyrazol-4-yl)methyl]-4,5-dihydro-2H-furo[2,3-g]indazole-7-carboxamide,
2-(4-chlorobenzyl)-N-(2-hydroxy-2-methylpropyl)-8-methyl-4,5-dihydro-2H-furo[2,3-g]indazole-7-carboxamide,
2-(4-chlorobenzyl)-N-[(1,1-dioxidotetrahydrothiophen-3-yl)methyl]-8-methyl-4,5-dihydro-2H-furo[2,3-g]indazole-7-carboxamide,
2-(4-chlorobenzyl)-8-methyl-N-[(1-methyl-1H-imidazol-2-yl)methyl]-4,5-dihydro-2H-furo[2,3-g]indazole-7-carboxamide,
2-(4-chlorobenzyl)-N-(1H-imidazol-2-ylmethyl)-N,8-dimethyl-4,5-dihydro-2H-furo[2,3-g]indazole-7-carboxamide,
(3-benzylazetidin-1-yl)[2-(4-chlorobenzyl)-8-methyl-4,5-dihydro-2H-furo[2,3-g]indazol-7-yl]methanone,
N-(3-amino-3-oxopropyl)-2-(4-chlorobenzyl)-8-methyl-4,5-dihydro-2H-furo[2,3-g]indazole-7-carboxamide,
2-(4-chlorobenzyl)-8-methyl-N-(pyrazolo[1,5-a]pyridin-3-ylmethyl)-4,5-dihydro-2H-furo[2,3-g]indazole-7-carboxamide,
2-(2-chlorobenzyl)-8-methyl-N-[(2RS)-tetrahydrofuran-2-ylmethyl]-4,5-dihydro-2H-furo[2,3-g]indazole-7-carboxamide,
2-(4-chlorobenzyl)-8-methyl-N-(6-oxopiperidin-3-yl)-4,5-dihydro-2H-furo[2,3-g]indazole-7-carboxamide,
3-azabicyclo[3.1.0]hexan-3-yl[2-(4-chlorobenzyl)-8-methyl-4,5-dihydro-2H-furo[2,3-g]indazol-7-yl]methanone,
2-(2-chlorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2RS)-tetrahydrofuran-2-ylmethyl]-4,5-dihydro-2H-furo[2,3-g]indazole-7-carboxamide,
2-(2-chlorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[(2R*)-tetrahydrofuran-2-ylmethyl]-4,5-dihydro-2H-furo[2,3-g]indazole-7-carboxamide,
2-(2-chloro-4-fluorobenzyl)-8-methyl-N-[2-(4-methylpiperazin-1-yl)ethyl]-4,5-dihydro-2H-furo[2,3-g]indazole-7-carboxamide,
2-(2-chloro-4-fluorobenzyl)-8-methyl-N-(1,2-oxazol-3-ylmethyl)-4,5-dihydro-2H-furo[2,3-g]indazole-7-carboxamide,
2-[(1RS)-1-(2-fluorophenyl)ethyl]-8-methyl-N-[(2S)-tetrahydrofuran-2-ylmethyl]-4,5-dihydro-2H-furo[2,3-g]indazole-7-carboxamide,
and a stereoisomer, a tautomer, an N-oxide, a hydrate, a solvate, or a salt thereof, and a mixture of same.

5. The use of a compound of general formula (I) according to claim 1, as an antagonist or inhibitor of G protein-coupled receptor 84 (GPR84).

6. A method of preparing a compound of general formula (I) according to claim 1, said method comprising the step of an intermediate compound of general formula (II): 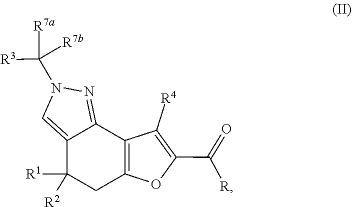
in which R is H, OH, OMe, or OEt and R¹, R², R³, R⁴, R^{7a} and R^{7b} are as defined for the compound of general formula (I) according to claim 1,
to react with a compound of general formula (III): 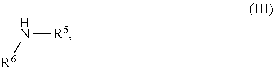
in which R⁵ and R⁶ are as defined for the compound of general formula (I) according to claim 1,
thereby giving a compound of general formula (I): 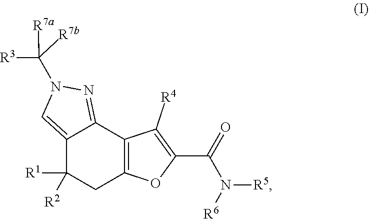
in which R¹, R², R³, R⁵, R⁶, R^{7a} and R^{7b} are as defined for the compound of general formula (I) according to claim 1.

7. A compound of general formula (II): 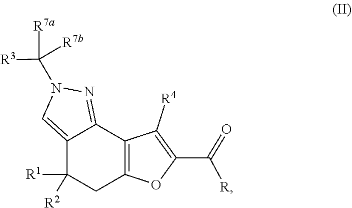
in which R is H, OH, OMe, or OEt and R¹, R², R³, R⁴, R^{7a} and R^{7b} are as defined for the compound of general formula (I) according to claim 1,

8. The use of a compound of general formula (II) 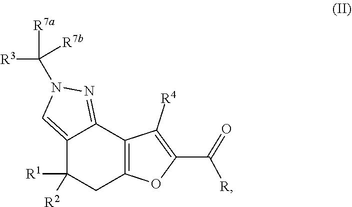
in which R is H, OH, OMe, or OEt and R¹, R², R³, R⁴, R^{7a} and R^{7b} are as defined for the compound of general formula (I) according to,
for the preparation of a compound of general formula (I) according to claim 1.
